(19) United States
(12) Patent Application Publication
(10) Pub. No.: US 2005/0043585 A1
(43) Pub. Date: Feb. 24, 2005

(54) Reticulated elastomeric matrices, their manufacture and use in implantable devices

(76) Inventors: Arindam Datta, Hillsborough, NJ (US); Craig Friedman, Westport, CT (US); Peter D. Costantino, Armonk, NY (US); Ian N. Askill, Colorado Springs, CO (US); Daniel Klempner, West Bloomfield, MI (US); Arthur H. Tinkelenberg, Brooklyn, NY (US); Aisa Sendijarevic, Troy, MI (US)

Correspondence Address: JONES DAY, 222 EAST 41ST ST, NEW YORK, NY, 10017, US

(21) Appl. No.: 10/749,742

(22) Filed: Dec. 30, 2003

Related U.S. Application Data
(60) Provisional application No. 60/437,955, filed on Jan. 3, 2003.
(60) Provisional application No. 60/471,520, filed on May 15, 2003.

(30) Foreign Application Priority Data
Oct. 23, 2003 (WO) PCT/US03/33750

(51) Int. Cl. A61B 1/00

(52) U.S. Cl. 600/153; 606/191

(57) ABSTRACT

This invention relates to biodurable, reticulated elastomeric matrices that are resiliently-compressible, their manufacture and uses including uses for implantable devices into or for topical treatment of patients, such as humans and other animals, for therapeutic, nutritional, or other useful purposes.

This application claims the benefit of U.S. provisional application No. 60/437,955, filed Jan. 3, 2003, U.S. provisional application No. 60/471,520, filed May 15, 2003, and International Application no. PCT/US03/33750, filed Oct. 23, 2003, the disclosure of each application being incorporated by reference herein in its entirety.

### FIELD OF THE INVENTION

This invention relates to reticulated elastomeric matrices, their manufacture and uses including uses for implantable devices into or for topical treatment of patients, such as humans and other animals, for therapeutic, nutritional, or other useful purposes. For these and other purposes the inventive products may be used alone or may be loaded with one or more deliverable substances.

### BACKGROUND OF THE INVENTION

Although porous implantable products are known that are intended to encourage tissue invasion in vivo, no known implantable device has been specifically designed or is available for the specific objective of being compressed for a delivery-device, e.g., catheter, endoscope or syringe, delivery to a biological site, being capable of expanding to occupy and remain in the biological site and being of a particular pore size such that it can become ingrown with tissue at that site to serve a useful therapeutic purpose.

Many porous, resiliently-compressible materials are produced from polyurethane foams formed by blowing during the polymerization process. In general such known processes are unattractive from the point of view of biodurability because undesirable materials that can produce adverse biological reactions are generated, for example carcinogens, cytotoxins and the like.

A number of polymers having varying degrees of biodurability are known, but commercially available materials either lack the mechanical properties needed to provide an implantable device that can be compressed for delivery-device delivery and can resiliently expand in situ, at the intended biological site, or lack sufficient porosity to induce adequate cellular ingrowth and proliferation. Some proposals of the art are further described below.

Greene, Jr., et al., in U.S. Pat. No. 6,165,193 (“Greene”), disclose a vascular implant formed of a compressible foam hydrogel that has a compressed configuration from which it is expansible into a configuration substantially conforming to the shape and size of a vascular malformation to be embolized. Greene's hydrogel lacks the mechanical properties to enable it to regain its size and shape in vivo were it to be compressed for catheter, endoscope or syringe delivery.

Brady et al., in U.S. Pat. No. 6,177,522 (“Brady '522”), disclose implantable porous polycarbonate polyurethane products comprising a polycarbonate that is disclosed to be a random copolymer of alkyl carbonates. Brady '522's crosslinked polymer comprises urea and biuret groups, when urea is present, and urethane and allophanate groups, when urethane is present.

Brady et al., in U.S. patent application Publication No. 2002/0072550 A1 (“Brady '550”), disclose implantable porous polyurethane products formed from a polyether or a polycarbonate linear long chain diol. Brady '550 does not broadly disclose a biostable porous polyether or polycarbonate polyurethane implant having isocyanurate linkages and a void content in excess of 85%. The diol of Brady '550 is disclosed to be free of tertiary carbon linkages. Additionally, Brady '550's diisocyanate is disclosed to be 4,4′-diphenylmethane diisocyanate containing less than 3% 2,4′-diphenylmethane diisocyanate. Furthermore, the final foamed polyurethane product of Brady '550 contains isocyanurate linkages and is not reticulated.

Brady et al., in U.S. patent application Publication No. 2002/0142413 A1 (“Brady '413”), disclose a tissue engineering scaffold for cell, tissue or organ growth or reconstruction, comprising a solvent-extracted, or purified, reticulated polyurethane, e.g. a polyether or a polycarbonate, having a high void content and surface area. Certain embodiments employ a blowing agent during polymerization for void creation. A minimal amount of cell window opening is effected by a hand press or by crushing and solvent extraction is used to remove the resulting residue. Accordingly, Brady '413 does not disclose a resiliently-compressible reticulated product or a process to make it.

Gilson et al., in U.S. Pat. No. 6,245,090 B1 (“Gilson”), disclose an open cell foam transcatheter occluding implant with a porous outer surface having good hysteresis properties, i.e., which, when used in a vessel that is continually expanding and contracting, is capable of expanding and contracting faster than the vessel. Additionally, Gilson's open cell foam is not reticulated.

Pinchuk, in U.S. Pat. Nos. 5,133,742 and 5,229,431 (“Pinchuk '742” and “Pinchuk '431”, respectively), discloses crack-resistant polyurethane for medical prostheses, implants, roofing insulators and the like. The polymer is a polycarbonate polyurethane polymer which is substantially completely devoid of ether linkages.

Szycher et al., in U.S. Pat. No. 5,863,267 (“Szycher”), disclose a biocompatible polycarbonate polyurethane with internal polysiloxane segments.

MacGregor, in U.S. Pat. No. 4,459,252, discloses cardiovascular prosthetic devices or implants comprising a porous surface and a network of interconnected interstitial pores below the surface in fluid flow communication with the surface pores.

Gunatillake et al., in U.S. Pat. No. 6,420,452 (“Gunatillake '452”), disclose a degradation resistant silicone-containing elastomeric polyurethane. Gunatillake et al., in U.S. Pat. No. 6,437,073 (“Gunatillake '073”), disclose a degradation-resistant silicone-containing polyurethane which is, furthermore, non-elastomeric.

Pinchuk, in U.S. Pat. No. 5,741,331 (“Pinchuk '331”), and its divisional U.S. Pat. Nos. 6,102,939 and 6,197,240, discloses supposed polycarbonate stability problems of microfiber cracking and breakage. Pinchuk '331 does not disclose a self-supporting, space-occupying porous element having three-dimensional resilient compressibility that can be catheter-, endoscope-, or syringe-introduced, occupy a biological site and permit cellular ingrowth and proliferation into the occupied volume.

Pinchuk et al., in U.S. patent application Publication No. 2002/0107330 A1 (“Pinchuk '330”), disclose a composition for implantation delivery of a therapeutic agent which comprises: a biocompatible block copolymer having an elastomeric block, e.g., polyolefin, and a thermoplastic block, e.g., styrene, and a therapeutic agent loaded into the block copolymer. The Pinchuk '330 compositions may lack adequate mechanical properties to provide a compressible catheter-, endoscope-, or syringe-introducible, resilient, space-occupying porous element that can occupy a biological site and permit cellular ingrowth and proliferation into the occupied volume.

Rosenbluth et al., in U.S. patent application Publication No. 2003/014075 A1 (“Rosenbluth”), disclose biomedical methods, materials, e.g., blood-absorbing, porous, expansible, super-strength hydrogels, and apparatus for deterring or preventing endoleaks following endovascular graft implantation. Rosenbluth does not disclose, e.g., polycarbonate polyurethane foams. Additionally, Rosenbluth's polymer foam is not reticulated.

Ma, in U.S. patent application Publication No. 2002/0005600 A1 (“Ma”), discloses a so-called reverse fabrication process of forming porous materials. For example, a solution of poly(lactide) in pyridine is added dropwise to a container of paraffin spheres, the pyridine is removed, then the paraffin is removed; a porous foam is disclosed to remain. Ma does not disclose, e.g., polycarbonate polyurethane foams. Further, Ma does not disclose a resiliently-compressible product.

Dereume et al., U.S. Pat. No. 6,309,413, relates to endoluminal grafts and discloses various methods of producing a 10-60 μm porous grafts, including elution of soluble particulates such as salts, sugar and hydrogels from polymers, and phase inversion. Tuch, in U.S. Pat. No. 5,820,917, discloses a blood-contacting medical device coated with a layer of water-soluble heparin, overlaid by a porous polymeric coating through which the heparin can elute. The porous polymer coating is prepared by methods such as phase inversion precipitation onto a stent yielding a product with a pore size of about 0.5- 10 μm. Dereume and Tuch disclose pore sizes that may be too small for effective cellular ingrowth and proliferation of uncoated substrates.

The above references do not disclose, e.g., an implantable device that is entirely suitable for delivery-device delivery, resilient recovery from that delivery, and long-term residence in a vascular malformation, with the therapeutic benefits, e.g., repair and regeneration, associated with appropriately-sized interconnected pores. Moreover, the above references do not disclose, e.g., such a device containing polycarbonate moieties.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the invention.

### SUMMARY OF THE INVENTION

The present invention solves the problem of providing a biological implantable device suitable for delivery-device, e.g., catheter, endoscope, arthoscope, laproscop, cystoscope or syringe, delivery to and long-term residence in a vascular and other sites in a patient, for example a mammal. To solve this problem, in one embodiment, the invention provides a biodurable, reticulated, resiliently-compressible elastomeric implantable device. In one embodiment, the implantable device is biodurable for at least 29 days. In another embodiment, the implantable device is biodurable for at least 2 months. In another embodiment, the implantable device is biodurable for at least 6 months. In another embodiment, the implantable device is biodurable for at least 12 months. In another embodiment, the implantable device is biodurable for at least 24 months. In another embodiment, the implantable device is biodurable for at least 5 years. In another embodiment, the implantable device is biodurable for longer than 5 years.

The structure, morphology and properties of the elastomeric matrices of this invention can be engineered or tailored over a wide range of performance by varying the starting materials and/or the processing conditions for different functional or therapeutic uses.

In one embodiment, the elastomeric matrix, as it becomes encapsulated and ingrown with cells and/or tissue, can play a less important role. In another embodiment, the encapsulated and ingrown elastomeric matrix occupies only a small amount of space, does not interfere with the function of the regrown cells and/or tissue, and has no tendency to migrate.

The inventive implantable device is reticulated, i.e., comprises an interconnected network of pores, either by being formed having a reticulated structure and/or undergoing a reticulation process. This provides fluid permeability throughout the implantable device and permits cellular ingrowth and proliferation into the interior of the implantable device. For this purpose, in one embodiment relating to vascular malformation applications and the like, the reticulated elastomeric matrix has pores with an average diameter or other largest transverse dimension of at least about 150 μm. In another embodiment, the reticulated elastomeric matrix has pores with an average diameter or other largest transverse dimension of greater than 250 μm. In another embodiment, the reticulated elastomeric matrix has pores with an average diameter or other largest transverse dimension of from about 275 μm to about 900 μm.

In one embodiment, an implantable device comprise a reticulated elastomeric matrix that is flexible and resilient and can recover its shape and most of its size after compression. In another embodiment, the inventive implantable devices have a resilient compressibility that allows the implantable device to be compressed under ambient conditions, e.g., at 25° C., from a relaxed configuration to a first, compact configuration for in vivo delivery via a delivery-device and to expand to a second, working configuration, in situ.

The present invention can provide truly reticulated, flexible, resilient, biodurable elastomeric matrix, suitable for long-term implantation and having sufficient porosity to encourage cellular ingrowth and proliferation, in vivo.

In another embodiment, the invention provides a process for producing a biodurable, flexible, reticulated, resiliently-compressible elastomeric matrix, suitable for implantation into patients, the process comprising forming pores in a well-characterized biodurable elastomer by a process free of undesirable residuals that does not substantially change the chemistry of the elastomer, to yield an elastomeric matrix having a reticulated structure that, when implanted in a patient, is biodurable for at least 29 days and has porosity providing fluid permeability throughout the elastomeric matrix and permitting cellular ingrowth and proliferation into the interior of the elastomeric matrix.

In another embodiment, the invention provides a process for producing an elastomeric matrix comprising a polymeric material having a reticulated structure, the process comprising:
a) fabricating a mold having surfaces defining a microstructural configuration for the elastomeric matrix;
b) charging the mold with a flowable polymeric material;
c) solidifying the polymeric material; and
d) removing the mold to yield the elastomeric matrix.
   The interconnecting interior passageways of the mold surfaces defining a desired microstructural configuration for the elastomeric matrix can be shaped, configured and dimensioned to define a self-supporting elastomeric matrix. In certain embodiments, the resultant elastomeric matrix has a reticulated structure. As described below, the fabricated mold can, in one embodiment, be a sacrificial mold that is removed to yield the reticulated elastomeric matrix. Such removal can be effected, for example, by melting, dissolving or subliming-away the sacrificial mold.

The substrate or sacrificial mold can comprise a plurality or multitude of solid or hollow beads or particles agglomerated, or interconnected, one with another at multiple points on each particle in the manner of a network. In one embodiment, the mold has a significant three-dimensional extent with multiple particles extending in each dimension. The particles of the mold may be interconnected using heat and/or pressure, e.g., by sintering or fusing, by means of an adhesive or solvent treatment, or by the application of a reduced pressure. In another embodiment, the polymeric material is contained within the interstices between the particles. In another embodiment, the polymeric material fills the interstices between the particles.

In one embodiment, the particles comprise a material having a relatively low melting point, for example, a hydrocarbon wax. In another embodiment, the particles comprise a material having water solubility, for example, an inorganic salt such as sodium chloride or calcium chloride, a sugar, such as sucrose, a starch, such as corn, potato, wheat, tapioca, manioc or rice starch, or mixtures thereof.

The polymeric material can comprise an elastomer. In another embodiment, the polymeric material can comprise a biodurable elastomer as described-herein. In another embodiment, the polymeric material can comprise a solvent-soluble biodurable elastomer whereby the flowable polymeric material can comprise a solution of the polymer. The solvent can then be removed or allowed to evaporate to solidify the polymeric material.

In another embodiment, the process is conducted to provide an elastomeric matrix configuration allowing cellular ingrowth and proliferation into the interior of the elastomeric matrix and the elastomeric matrix is implantable into a patient, as described herein. Without being bound by any particular theory, having a high void content and a high degree of reticulation is thought to allow the implantable devices to be completely ingrown and proliferated with cells including tissues such as fibrous tissues.

In another embodiment, the invention provides a process for producing an elastomeric matrix having a reticulated structure, the process comprising:
a) coating a reticulated foam template with a flowable resistant material, optionally a thermoplastic polymer or a wax;
b) exposing a coated surface of the foam template;
c) removing the foam template to yield a casting of the reticulated foam template;
d) coating the casting with an elastomer in a flowable state to form an elastomeric matrix;
e) exposing a surface of the casting; and
f) removing the casting to yield a reticulated polyurethane elastomeric matrix comprising the elastomer.

In another embodiment, the invention provides a lyophilization process for producing an elastomeric matrix having a reticulated structure, the process comprising:
a) forming a solution comprising a solvent-soluble biodurable elastomer in a solvent;
b) at least partially solidifying the solution to form a solid, optionally by cooling the solution; and
c) removing the non-polymeric material, optionally by subliming the solvent from the solid under reduced pressure, to provide an at least partially reticulated elastomeric matrix comprising the elastomer.

In another embodiment, the invention provides a polymerization process for preparing a reticulated elastomeric matrix, the process comprising admixing:
a) a polyol component,
b) an isocyanate component,
c) a blowing agent,
d) optionally, a crosslinking agent,
e) optionally, a chain extender,
f) optionally, at least one catalyst,
g) optionally, a surfactant, and
h) optionally, a viscosity modifier;
   to provide a crosslinked elastomeric matrix and reticulating the elastomeric matrix by a reticulation process to provide the reticulated elastomeric matrix. The ingredients are present in quantities the elastomeric matrix is prepared and under conditions to (i) provide a crosslinked resiliently-compressible biodurable elastomeric matrix, (ii) control formation of biologically undesirable residues, and (iii) reticulate the foam by a reticulation process, to provide the reticulated elastomeric matrix.

In another embodiment, the invention provides a lyophilization process for preparing a reticulated elastomeric matrix comprising lyophilizing a flowable polymeric material. In another embodiment, the polymeric material comprises a solution of a solvent-soluble biodurable elastomer in a solvent. In another embodiment, the flowable polymeric material is subjected to a lyophilization process comprising solidifying the flowable polymeric material to form a solid, e.g., by cooling a solution, then removing the non-polymeric material, e.g., by subliming the solvent from the solid under reduced pressure, to provide an at least partially reticulated elastomeric matrix. In another embodiment, a solution of a biodurable elastomer in a solvent is substantially, but not necessarily completely, solidified, then the solvent is sublimed from that material to provide an at least partially reticulated elastomeric matrix. In another embodiment, the temperature to which the solution is cooled is below the freezing temperature of the solution. In another embodiment, the temperature to which the solution is cooled is above the apparent glass transition temperature of the solid and below the freezing temperature of the solution.

In another embodiment, the invention provides a process for preparing a reticulated composite elastomeric implantable device for implantation into a patient, the process comprising surface coating or endoporously coating a biodurable reticulated elastomeric matrix with a coating material selected to encourage cellular ingrowth and proliferation. The coating material can, for example, comprise a foamed coating of a biodegradable material, optionally, collagen, fibronectin, elastin, hyaluronic acid and mixtures thereof. Alternatively, the coating comprises a biodegradable polymer and an inorganic component.

In another embodiment, the invention provides a process for preparing a reticulated composite elastomeric implantable device useful for implantation into a patient, the process comprising surface coating or endoporously coating or impregnating a reticulated biodurable elastomer. This coating or impregnating material can, for example, comprise polyglycolic acid (“PGA”), polylactic acid (“PLA”), polycaprolactic acid (“PCL”), poly-p-dioxanone (“PDO”), PGA/PLA copolymers, PGA/PCL copolymers, PGA/PDO copolymers, PLA/PCL copolymers, PLA/PDO copolymers, PCL/PDO copolymers or combinations of any two or more of the foregoing. Another embodiment involves surface coating or surface fusion, wherein the porosity of the surface is altered.

In another embodiment, the invention provides a method for treating an vascular malformation in a patient, such as an animal, the method comprising:
a) compressing the herein-described inventive implantable device from a relaxed configuration to a first, compact configuration;
b) delivering the compressed implantable device to the in vivo site of the vascular malformation via a delivery-device; and
c) allowing the implantable device to resiliently recover and expand to a second, working configuration at the in vivo site.

### BRIEF DESCRIPTION OF THE DRAWINGS

Some embodiments of the invention, and of making and using the invention, as well as the best mode contemplated of carrying out the invention, are described in detail below, which description is to be read with and in the light of the foregoing description, by way of example, with reference to the accompanying drawings, in which like reference characters designate the same or similar elements throughout the several views, and in which:

FIG. 1 is a schematic view showing one possible morphology for a portion of the microstructure of one embodiment of a porous biodurable elastomeric product according to the invention;

FIG. 2 is a schematic block flow diagram of a process for preparing a porous biodurable elastomeric implantable device according to the invention;

FIG. 3 is a schematic block flow diagram of a sacrificial molding process for preparing a reticulated biodurable elastomeric implantable device according to the invention;

FIG. 4 is a schematic view of an apparatus for performing the sacrificial molding process illustrated in FIG. 3;

FIG. 5 is a schematic block flow diagram, with accompanying product sectional views, of a double lost wax process for preparing a reticulated biodurable elastomeric implantable device according to the invention;

FIG. 6 is a scanning electron micrograph image of the reticulated elastomeric implantable device prepared in Example 3; and

FIG. 7 is a histology slide of a reticulated implantable device prepared according to Example 3 following removal after 14 day implantation in the subcutaneous tissue of a Sprague-Dawley rat.

### DETAILED DESCRIPTION OF THE INVENTION

Certain embodiments of the invention comprise reticulated biodurable elastomer products, which are also compressible and exhibit resilience in their recovery, that have a diversity of applications and can be employed, by way of example, in management of vascular malformations, such as for aneurysm control, arterio venous malfunction, arterial embolization or other vascular abnormalities, or as substrates for pharmaceutically-active agent, e.g., for drug delivery. Thus, as used herein, the term “vascular malformation” includes but is not limited to aneurysms, arterio venous malfunctions, arterial embolizations and other vascular abnormalities. Other embodiments involve reticulated biodurable elastomer products for in vivo delivery via catheter, endoscope, arthoscope, laproscope, cystoscope, syringe or other suitable delivery-device and can be satisfactorily implanted or otherwise exposed to living tissue and fluids for extended periods of time, for example, at least 29 days.

There is a need in medicine, as recognized by the present invention, for innocuous implantable devices that can be delivered to an in vivo patient site, for example a site in a human patient, that can occupy that site for extended periods of time without being harmful to the host. In one embodiment, such implantable devices can also eventually become integrated, e.g., ingrown with tissue. Various implants have long been considered potentially useful for local in situ delivery of biologically active agents and more recently have been contemplated as useful for control of endovascular conditions including potentially life-threatening conditions such as cerebral and aortic abdominal aneurysms, arterio venous malfunction, arterial embolization or other vascular abnormalities.

It would be desirable to have an implantable system which, e.g., can optionally reduce blood flow due to the pressure drop caused by additional resistance, optionally cause immediate thrombotic response leading to clot formation, and eventually lead to fibrosis, i.e., allow for and stimulate natural cellular ingrowth and proliferation into vascular malformations and the void space of implantable devices located in vascular malformations, to stabilize and possibly seal off such features in a biologically sound, effective and lasting manner. However, prior to the present invention, materials and products meeting all the requirements of such an implantable system have not been available.

Broadly stated, certain embodiments of the reticulated biodurable elastomeric products of the invention comprise, or are largely, if not entirely, constituted by a highly permeable, reticulated matrix formed of a biodurable polymeric elastomer that is resiliently-compressible so as to regain its shape after delivery to a biological site. In one embodiment, the elastomeric matrix is chemically well-characterized. In another embodiment, the elastomeric matrix is physically well-characterized. In another embodiment, the elastomeric matrix is chemically and physically well-characterized.

Certain embodiments of the invention can support cell growth and permit cellular ingrowth and proliferation in vivo and are useful as in vivo biological implantable devices, for example, for treatment of vasculature problems that may be used in vitro or in vivo to provide a substrate for cellular propagation.

In one embodiment, the reticulated elastomeric matrix of the invention facilitates tissue ingrowth by providing a surface for cellular attachment, migration, proliferation and/or coating (e.g., collagen) deposition. In another embodiment, any type of tissue can grow into an implantable device comprising a reticulated elastomeric matrix of the invention, including, by way of example, epithelial tissue (which includes, e.g., squamous, cuboidal and columnar epithelial tissue), connective tissue (which includes, e.g., areolar tissue, dense regular and irregular tissue, reticular tissue, adipose tissue, cartilage and bone), and muscle tissue (which includes, e.g., skeletal, smooth and cardiac muscle), or any combination thereof, e.g., fibrovascular tissue. In another embodiment of the invention, an implantable device comprising a reticulated elastomeric matrix of the invention can have tissue ingrowth substantially throughout the volume of its interconnected pores.

In one embodiment, the invention comprises an implantable device having sufficient resilient compressibility to be delivered by a “delivery-device”, i.e., a device with a chamber for containing an elastomeric implantable device while it is delivered to the desired site then released at the site, e.g., using a catheter, endoscope, arthoscope, laproscope, cystoscope or syringe. In another embodiment, the thus-delivered elastomeric implantable device substantially regains its shape after delivery to a biological site and has adequate biodurability and biocompatibility characteristics to be suitable for long-term implantation.

The structure, morphology and properties of the elastomeric matrices of this invention can be engineered or tailored over a wide range of performance by varying the starting materials and/or the processing conditions for different functional or therapeutic uses.

Without being bound by any particular theory, it is thought that an aim of the invention, to provide a light-weight, durable structure that can fill a biological volume or cavity and containing sufficient porosity distributed throughout the volume, can be fulfilled by permitting one or more of: occlusion and embolization, cellular ingrowth and proliferation, tissue regeneration, cellular attachment, drug delivery, enzymatic action by immobilized enzymes, and other useful processes as described herein including, in particular, the copending applications.

In one embodiment, elastomeric matrices of the invention have sufficient resilience to allow substantial recovery, e.g., to at least about 50% of the size of the relaxed configuration in at least one dimension, after being compressed for implantation in the human body, for example, a low compression set, e.g., at 25° C. or 37° C., and sufficient strength and flow-through for the matrix to be used for controlled release of pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment, elastomeric matrices of the invention have sufficient resilience to allow recovery to at least about 90% of the size of the relaxed configuration in at least one dimension after being compressed for implantation in the human body.

In the present application, the term “biodurable” describes elastomers and other products that are stable for extended periods of time in a biological environment. Such products should not exhibit significant symptoms of breakdown or degradation, erosion or significant deterioration of mechanical properties relevant to their employment when exposed to biological environments for periods of time commensurate with the use of the implantable device. The period of implantation may be weeks, months or years; the lifetime of a host product in which the elastomeric products of the invention are incorporated, such as a graft or prosthetic; or the lifetime of a patient host to the elastomeric product. In one embodiment, the desired period of exposure is to be understood to be at least about 29 days. In another embodiment, the desired period of exposure is to be understood to be at least 29 days.

In one embodiment, biodurable products of the invention are also biocompatible. In the present application, the term “biocompatible” means that the product induces few, if any, adverse biological reactions when implanted in a host patient. Similar considerations applicable to “biodurable” also apply to the property of “biocompatibility”.

An intended biological environment can be understood to in vivo, e.g., that of a patient host into which the product is implanted or to which the product is topically applied, for example, a mammalian host such as a human being or other primate, a pet or sports animal, a livestock or food animal, or a laboratory animal. All such uses are contemplated as being within the scope of the invention. As used herein, a “patient” is an animal. In one embodiment, the animal is a bird, including but not limited to a chicken, turkey, duck, goose or quail, or a mammal. In another embodiment, the animal is a mammal, including but not limited to a cow, horse, sheep, goat, pig, cat, dog, mouse, rat, hamster, rabbit, guinea pig, monkey and a human. In another embodiment, the animal is a primate or a human. In another embodiment, the animal is a human.

In one embodiment, structural materials for the inventive porous elastomers are synthetic polymers, especially, but not exclusively, elastomeric polymers that are resistant to biological degradation, for example polycarbonate polyurethanes, polyether polyurethanes, polysiloxanes and the like. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The reticulated biodurable elastomeric products of the invention can be described as having a “macrostructure” and a “microstructure”, which terms are used herein in the general senses described in the following paragraphs.

The “macrostructure” refers to the overall physical characteristics of an article or object formed of the biodurable elastomeric product of the invention, such as: the outer periphery as described by the geometric limits of the article or object, ignoring the pores or voids; the “macrostructural surface area” which references the outer surface areas as though the pores were filled and ignores the surface areas within the pores; the “macrostructural volume” or simply the “volume” occupied by the article or object which is the volume bounded by the macrostructural, or simply “macro” surface area; and the “bulk density” which is the weight per unit volume of the article or object itself as distinct from the density of the structural material.

The “microstructure” refers to the features of the interior structure of the biodurable elastomeric material from which the inventive products are constituted such as pore dimensions; pore surface area, being the total area of the material surfaces in the pores; and the configuration of the struts and intersections that constitute the solid structure of certain embodiments of the inventive elastomeric product.

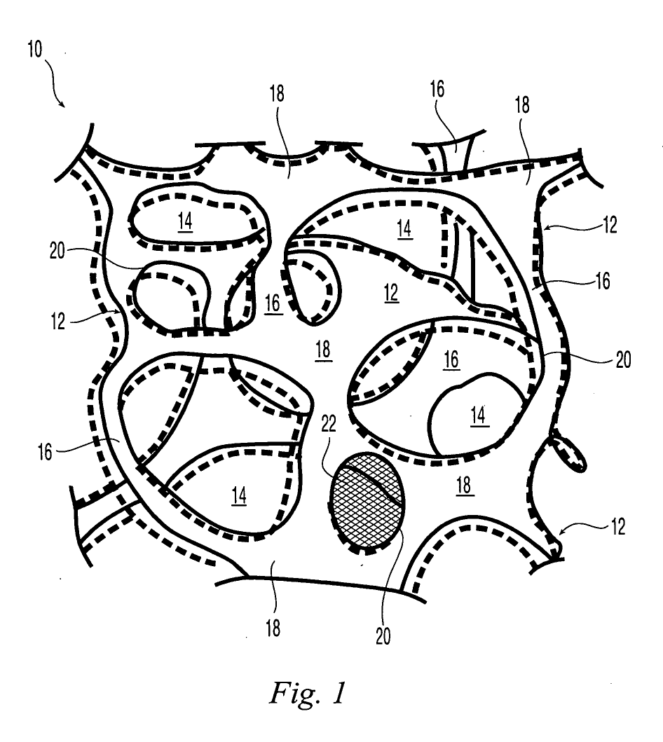

Referring to FIG. 1, what is shown for convenience is a schematic depiction of the particular morphology of a reticulated foam. FIG. 1 is a convenient way of illustrating some of the features and principles of the microstructure of some embodiments of the invention. This figure is not intended to be an idealized depiction of an embodiment of, nor is it a detailed rendering of a particular embodiment of the elastomeric products of the invention. Other features and principles of the microstructure will be apparent from the present specification, or will be apparent from one or more of the inventive processes for manufacturing porous elastomeric products that are described herein.

Morphology

Described generally, the microstructure of the illustrated porous biodurable elastomeric matrix 10, which may, inter alia, be an individual element having a distinct shape or an extended, continuous or amorphous entity, comprises a reticulated solid phase 12 formed of a suitable biodurable elastomeric material and interspersed therewithin, or defined thereby, a continuous interconnected void phase 14, the latter being a principle feature of a reticulated structure.

In one embodiment, the elastomeric material of which elastomeric matrix 10 is constituted may be a mixture or blend of multiple materials. In another embodiment, the elastomeric material is a single synthetic polymeric elastomer such as will be described in more detail below.

Void phase 14 will usually be air- or gas-filled prior to use. During use, void phase 14 will in many but not all cases become filled with liquid, for example, with biological fluids or body fluids.

Solid phase 12 of elastomeric matrix 10, as shown in FIG. 1, has an organic structure and comprises a multiplicity of relatively thin struts 16 that extend between and interconnect a number of intersections 18. The intersections 18 are substantial structural locations where three or more struts 16 meet one another. Four or five or more struts 16 may be seen to meet at an intersection 18 or at a location where two intersections 18 can be seen to merge into one another. In one embodiment, struts 16 extend in a three-dimensional manner between intersections 18 above and below the plane of the paper, favoring no particular plane. Thus, any given strut 16 may extend from an intersection 18 in any direction relative to other struts 16 that join at that intersection 18. Struts 16 and intersections 18 may have generally curved shapes and define between them a multitude of pores 20 or interstitial spaces in solid phase 12. Struts 16 and intersections 18 form an interconnected, continuous solid phase.

As illustrated in FIG. 1, the structural components of the solid phase 12 of elastomeric matrix 10, namely struts 16 and intersections 18, may appear to have a somewhat laminar configuration as though some were cut from a single sheet, it will be understood that this appearance may in part be attributed to the difficulties of representing complex three-dimensional structures in a two dimensional figure. Struts 16 and intersections 18 may have, and in many cases will have, non-laminar shapes including circular, elliptical and non-circular cross-sectional shapes and cross sections that may vary in area along the particular structure, for example, they may taper to smaller and/or larger cross sections while traversing along their longest dimension.

A small number of pores 20 may have a cell wall of structural material also called a “window” or “window pane” such as cell wall 22. Such cell walls are undesirable to the extent that they obstruct the passage of fluid and/or propagation and proliferation of tissues through pores 20. Cell walls 22 may, in one embodiment, be removed in a suitable process step, such as reticulation as discussed below.

Except for boundary terminations at the macrostructural surface, in the embodiment shown in FIG. 1 solid phase 12 of elastomeric matrix 10 comprises few, if any, free-ended, dead-ended or projecting “strut-like” structures extending from struts 16 or intersections 18 but not connected to another strut or intersection.

However, in an alternative embodiment, solid phase 12 can be provided with a plurality of such fibrils (not shown), e.g., from about 1 to about 5 fibrils per strut 16 or intersection 18. In some applications, such fibrils may be useful, for example, for the additional surface area they provide. However, such projecting or protuberant structures may impede or restrict flow through pores 20.

Struts 16 and intersections 18 can be considered to define the shape and configuration of the pores 20 that make up void phase 14 (or vice versa). Many of pores 20, in so far as they may be discretely identified, open into and communicate with at least two other pores 20. At intersections 18, three or more pores 20 may be considered to meet and intercommunicate. In certain embodiments, void phase 14 is continuous or substantially continuous throughout elastomeric matrix 10, meaning that there are few if any closed cell pores 20. Such closed cell pores 20 represent loss of useful volume and may obstruct access of useful fluids to interior strut and intersection structures 16 and 18 of elastomeric matrix 10.

In one embodiment, such closed cell pores 20, if present, comprise less than about 15% of the volume of elastomeric matrix 10. In another embodiment, such closed cell pores 20, if present, comprise less than about 5% of the volume of elastomeric matrix 10. In another embodiment, such closed cell pores 20, if present, comprise less than about 2% of the volume of elastomeric matrix 10. The presence of closed cell pores 20 can be noted by their influence in reducing the volumetric flow rate of a fluid through elastomeric matrix 10 and/or as a reduction in cellular ingrowth and proliferation into elastomeric matrix 10.

In another embodiment, elastomeric matrix 10 is reticulated. In another embodiment, elastomeric matrix 10 is substantially reticulated. In another embodiment, elastomeric matrix 10 is fully reticulated. In another embodiment, elastomeric matrix 10 has many cell walls 22 removed. In another embodiment, elastomeric matrix 10 has most cell walls 22 removed. In another embodiment, elastomeric matrix 10 has substantially all cell walls 22 removed.

In another embodiment, solid phase 12, which may be described as reticulated, comprises a continuous network of solid structures, such as struts 16 and intersections 18, without any significant terminations, isolated zones or discontinuities, other than at the boundaries of the elastomeric matrix, in which network a hypothetical line may be traced entirely through the material of solid phase 12 from one point in the network to any other point in the network.

In another embodiment, void phase 14 is also a continuous network of interstitial spaces, or intercommunicating fluid passageways for gases or liquids, which fluid passageways extend throughout and are defined by (or define) the structure of solid phase 12 of elastomeric matrix 10 and open into all its exterior surfaces. In other embodiments, as described above, there are only a few, substantially no, or no occlusions or closed cell pores 20 that do not communicate with at least one other pore 20 in the void network. Also in this void phase network, a hypothetical line may be traced entirely through void phase 14 from one point in the network to any other point in the network.

In concert with the objectives of the invention, in one embodiment the microstructure of elastomeric matrix 10 is constructed to permit or encourage cellular adhesion to the surfaces of solid phase 12, neointima formation thereon and cellular and tissue ingrowth and proliferation into pores 20 of void phase 14, when elastomeric matrix 10 resides in suitable in vivo locations for a period of time.

In another embodiment, such cellular or tissue ingrowth and proliferation, which may for some purposes include fibrosis, can occur or be encouraged not just into exterior layers of pores 20, but into the deepest interior of and throughout elastomeric matrix 10. Thus, in this embodiment, the space occupied by elastomeric matrix 10 becomes entirely filled by the cellular and tissue ingrowth and proliferation in the form of fibrotic, scar or other tissue except, of course, for the space occupied by the elastomeric solid phase 12. In another embodiment, the inventive implantable device functions so that ingrown tissue is kept vital, for example, by the prolonged presence of a supportive microvasculature.

To this end, particularly with regard to the morphology of void phase 14, in one embodiment elastomeric matrix 10 is reticulated with open interconnected pores. Without being bound by any particular theory, this is thought to permit natural irrigation of the interior of elastomeric matrix 10 with bodily fluids, e.g., blood, even after a cellular population has become resident in the interior of elastomeric matrix 10 so as to sustain that population by supplying nutrients thereto and removing waste products therefrom. In another embodiment, elastomeric matrix 10 is reticulated with open interconnected pores of a particular size range. In another embodiment, elastomeric matrix 10 is reticulated with open interconnected pores with a distribution of size ranges.

It is intended that the various physical and chemical parameters of elastomeric matrix 10 including in particular the parameters to be described below, be selected to encourage cellular ingrowth and proliferation according to the particular application for which an elastomeric matrix 10 is intended.

It will be understood that such constructions of elastomeric matrix 10 that provide interior cellular irrigation will be fluid permeable and may also provide fluid access through and to the interior of the matrix for purposes other than cellular irrigation, for example, for elution of pharmaceutically-active agents, e.g., a drug, or other biologically useful materials. Such materials may optionally be secured to the interior surfaces of elastomeric matrix 10.

In another embodiment of the invention, gaseous phase 12 can be filled or contacted with a deliverable treatment gas, for example, a sterilant such as ozone or a wound healant such as nitric oxide, provided that the macrostructural surfaces are sealed, for example by a bioabsorbable membrane to contain the gas within the implanted product until the membrane erodes releasing the gas to provide a local therapeutic or other effect.

Useful embodiments of the invention include structures that are somewhat randomized, as shown in FIG. 1 where the shapes and sizes of struts 16, intersections 18 and pores 20 vary substantially, and more ordered structures which also exhibit the described features of three-dimensional interpenetration of solid and void phases, structural complexity and high fluid permeability. Such more ordered structures can be produced by the processes of the invention as will be fturther described below.

Porosity

Void phase 14 may comprise as little as 50% by volume of elastomeric matrix 10, referring to the volume provided by the interstitial spaces of elastomeric matrix 10 before any optional interior pore surface coating or layering is applied. In one embodiment, the volume of void phase 14, as just defined, is from about 70% to about 99% of the volume of elastomeric matrix 10. In another embodiment, the volume of void phase 14 is from about 80% to about 98% of the volume of elastomeric matrix 10. In another embodiment, the volume of void phase 14 is from about 90% to about 98% of the volume of elastomeric matrix 10.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. As used herein, the “average diameter or other largest transverse dimension” refers to the number average diameter, for spherical or substantially spherical pores, or to the number average largest transverse dimension, for non-spherical pores.

In one embodiment relating to vascular malformation applications and the like, to encourage cellular ingrowth and proliferation and to provide adequate fluid permeability, the average diameter or other largest transverse dimension of pores 20 is at least about 100 μm. In another embodiment, the average diameter or other largest transverse dimension of pores 20 is at least about 150 μm. In another embodiment, the average diameter or other largest transverse dimension of pores 20 is at least about 250 μm. In another embodiment, the average diameter or other largest transverse dimension of pores 20 is greater than about 250 μm. In another embodiment, the average diameter or other largest transverse dimension of pores 20 is greater than 250 μm. In another embodiment, the average diameter or other largest transverse dimension of pores 20 is at least about 275 μm. In another embodiment, the average diameter or other largest transverse dimension of pores 20 is greater than about 275 μm. In another embodiment, the average diameter or other largest transverse dimension of pores 20 is greater than 275 μm. In another embodiment, the average diameter or other largest transverse dimension of pores 20 is at least about 300 μm. In another embodiment, the average diameter or other largest transverse dimension of pores 20 is greater than about 300 μm. In another embodiment, the average diameter or other largest transverse dimension of pores 20 is greater than 300 μm.

In another embodiment relating to vascular malformation applications and the like, the average diameter or other largest transverse dimension of pores 20 is not greater than about 900 μm. In another embodiment, the average diameter or other largest transverse dimension of pores 20 is not greater than about 850 μm. In another embodiment, the average diameter or other largest transverse dimension of pores 20 is not greater than about 800 μm. In another embodiment, the average diameter or other largest transverse dimension of pores 20 is not greater than about 700 μm. In another embodiment, the average diameter or other largest transverse dimension of pores 20 is not greater than about 600 μm. In another embodiment, the average diameter or other largest transverse dimension of pores 20 is not greater than about 500 μm.

In another embodiment relating to vascular malformation applications and the like, the average diameter or other largest transverse dimension of pores 20 is from about 100 μm to about 900 μm. In another embodiment, the average diameter or other largest transverse dimension of pores 20 is from about 100 μm to about 850 μm. In another embodiment, the average diameter or other largest transverse dimension of pores 20 is from about 100 μm to about 800 μm. In another embodiment, the average diameter or other largest transverse dimension of pores 20 is from about 100 μm to about 700 μm. In another embodiment, the average diameter or other largest transverse dimension of pores 20 is from about 150 μm to about 600 μm. In another embodiment, the average diameter or other largest transverse dimension of pores 20 is from about 200 μm to about 500 μm. In another embodiment, the average diameter or other largest transverse dimension of pores 20 is greater than about 250 μm to about 900 μm. In another embodiment, the average diameter or other largest transverse dimension of pores 20 is greater than about 250 μm to about 850 μm. In another embodiment, the average diameter or other largest transverse dimension of pores 20 is greater than about 250 μm to about 800 μm. In another embodiment, the average diameter or other largest transverse dimension of pores 20 is greater than about 250 μm to about 700 μm. In another embodiment, the average diameter or other largest transverse dimension of pores 20 is greater than about 250 μm to about 600 μm. In another embodiment, the average diameter or other largest transverse dimension of pores 20 is from about 275 μm to about 900 μm. In another embodiment, the average diameter or other largest transverse dimension of pores 20 is from about 275 μm to about 850 μm. In another embodiment, the average diameter or other largest transverse dimension of pores 20 is from about 275 μm to about 800 μm. In another embodiment, the average diameter or other largest transverse dimension of pores 20 is from about 275 μm to about 700 μm. In another embodiment, the average diameter or other largest transverse dimension of pores 20 is from about 275 μm to about 600 μm.

Pore size, pore size distribution, surface area, gas permeability and liquid permeability can be measured by conventional methods known to those in the art. Some measurement methods are summarized, e.g., by A. Jena and K. Gupta in “Advanced Technology for Evaluation of Pore Structure Characteristics of Filtration Media to Optimize Their Design and Performance”, available at www.pmjapp.com/papers/index.html, and in the publication “A Novel Mercury Free Technique for Determination of Pore Volume, Pore Size and Liquid Permeability.” Apparatus that can be used to conduct such determinations includes the Capillary Flow Porometer and the Liquid Extrusion Porosimeter, each available from Porous Materials, Inc. (Ithaca, N.Y.).

Size and Shape

Elastomeric matrix 10 can be readily fabricated in any desired size and shape. It is a benefit of the invention that elastomeric matrix 10 is suitable for mass production from bulk stock by subdividing such bulk stock, e.g., by cutting, die punching, laser slicing, or compression molding. In one embodiment, subdividing the bulk stock can be done using a heated surface. It is a further benefit of the invention that the shape and configuration of elastomeric matrix 10 may vary widely and can readily be adapted to desired anatomical morphologies.

The size, shape, configuration and other related details of elastomeric matrix 10 can be either customized to a particular application or patient or standardized for mass production. However, economic considerations favor standardization. To this end, elastomeric matrix 10 can be embodied in a kit comprising elastomeric implantable device pieces of different sizes and shapes. Also, as discussed elsewhere in the present specification and as is disclosed in the copending applications, multiple, e.g. two, three or four, individual elastomeric matrices 10 can be used as an implantable device system for a single target biological site, being sized or shaped or both sized and shaped to function cooperatively for treatment of an individual target site.

The practitioner performing the procedure, who may be a surgeon or other medical or veterinary practitioner, researcher or the like, may then choose one or more implantable devices from the available range to use for a specific treatment, for example, as is described in the copending applications.

By way of example, the minimum dimension of elastomeric matrix 10 may be as little as 1 mm and the maximum dimension as much as 100 mm or even greater. However, in one embodiment it is contemplated that an elastomeric matrix 10 of such dimension intended for implantation would have an elongated shape, such as the shapes of cylinders, rods, tubes or elongated prismatic forms, or a folded, coiled, helical or other more compact configuration. Comparably, a dimension as small as 1 mm can be a transverse dimension of an elongated shape or of a ribbon or sheet-like implantable device.

In an alternative embodiment, an elastomeric matrix 10 having a spherical, cubical, tetrahedral, toroidal or other form having no dimension substantially elongated when compared to any other dimension and with a diameter or other maximum dimension of from about 1 mm to about 100 mm may have utility, for example, for vascular occlusion. In another embodiment, the elastomeric matrix 10 having such a form has a diameter or other maximum dimension from about 3 mm to about 20 mm.

For most implantable device applications, macrostructural sizes of elastomeric matrix 10 include the following embodiments: compact shapes such as spheres, cubes, pyramids, tetrahedrons, cones, cylinders, trapezoids, parallelepipeds, ellipsoids, fusiforms, tubes or sleeves, and many less regular shapes having transverse dimensions of from about 1 mm to about 200 mm (In another embodiment, these transverse dimensions are from about 5 mm to about 100 mm.); and sheet- or strip-like shapes having a thickness of from about 1 mm to about 20 mm (In another embodiment, these thickness are from about 1 mm to about 5 mm.) and lateral dimensions of from about 5 mm to about 200 mm (In another embodiment, these, lateral dimensions are from about 10 mm to about 100 mm.).

For treatment of vascular malformations, it is an advantage of the invention that the implantable elastomeric matrix elements can be effectively employed without any need to closely conform to the configuration of the vascular malformation, which may often be complex and difficult to model. Thus, in one embodiment, the implantable elastomeric matrix elements of the invention have significantly different and simpler configurations, for example, as described in the copending applications.

Furthermore, in one embodiment, the implantable device of the present invention, or implantable devices if more than one is used, should not completely fill the aneurysm or other vascular malformation even when fully expanded in situ. In one embodiment, the fully expanded implantable device(s) of the present invention are smaller in a dimension than the vascular malformation and provide sufficient space within the vascular malformation to ensure vascularization, cellular ingrowth and proliferation, and for passage of blood to the implantable device. In another embodiment, the fully expanded implantable device(s) of the present invention are substantially the same in a dimension as the vascular malformation. In another embodiment, the fully expanded implantable device(s) of the present invention are larger in a dimension than the vascular malformation. In another embodiment, the fully expanded implantable device(s) of the present invention are smaller in volume than the vascular malformation. In another embodiment, the fully expanded implantable device(s) of the present invention are substantially the same volume as the vascular malformation. In another embodiment, the fully expanded implantable device(s) of the present invention are larger in volume than the vascular malformation.

Some useful implantable device shapes may approximate a portion of the target vascular malformation. In one embodiment, the implantable device is shaped as relatively simple convex, dish-like or hemispherical or hemi-ellipsoidal shape and size that is appropriate for treating multiple different sites in different patients.

It is contemplated, in another embodiment, that even when their pores become filled with biological fluids, bodily fluids and/or tissue in the course of time, such implantable devices for vascular malformation applications and the like do not entirely fill the biological site in which they reside and that an individual implanted elastomeric matrix 10 will, in many cases, although not necessarily, have a volume of no more than 50% of the biological site within the entrance thereto. In another embodiment, an individual implanted elastomeric matrix 10 will have a volume of no more than 75% of the biological site within the entrance thereto. In another embodiment, an individual implanted elastomeric matrix 10 will have a volume of no more than 95% of the biological site within the entrance thereto.

In another embodiment, when their pores become filled with biological fluids, bodily fluids and/or tissue in the course of time, such implantable devices for vascular malformation applications and the like substantially fill the biological site in which they reside and an individual implanted elastomeric matrix 10 will, in many cases, although not necessarily, have a volume of no more than about 100% of the biological site within the entrance thereto. In another embodiment, an individual implanted elastomeric matrix 10 will have a volume of no more than about 98% of the biological site within the entrance thereto. In another embodiment, an individual implanted elastomeric matrix 10 will have a volume of no more than about 102% of the biological site within the entrance thereto.

In another embodiment, when their pores become filled with biological fluids, bodily fluids and/or tissue in the course of time, such implantable devices for vascular malformation applications and the like over-fill the biological site in which they reside and an individual implanted elastomeric matrix 10 will, in many cases, although not necessarily, have a volume of more than about 105% of the biological site within the entrance thereto. In another embodiment, an individual implanted elastomeric matrix 10 will have a volume of more than about 125% of the biological site within the entrance thereto. In another embodiment, an individual implanted elastomeric matrix 10 will have a volume of more than about 150% of the biological site within the entrance thereto.

A further alternative morphology for elastomeric matrix 10 comprises emboli or particles useful for end vessel occlusion, capillary closure and other purposes, which emboli have a generally spherical or other desired shape, and an average size of less than about 1 mm, for example from about 10 μm to about 500 μm. In another embodiment, emboli have a generally spherical or other desired shape, and an average size with a narrow distribution of less than about 1 mm. Such emboli may be porous, as elastomeric matrix 10 has generally been described herein, solid or hollow.

Well-Characterized Elastomers and Elastomeric Implantable Devices

Elastomers for use as the structural material of elastomeric matrix 10 alone, or in combination in blends or solutions, are, in one embodiment, well-characterized synthetic elastomeric polymers having suitable mechanical properties which have been sufficiently characterized with regard to chemical, physical or biological properties as to be considered biodurable and suitable for use as in vivo implantable devices in patients, particularly in mammals and especially in humans. In another embodiment, elastomers for use as the structural material of elastomeric matrix 10 are sufficiently characterized with regard to chemical, physical and biological properties as to be considered biodurable and suitable for use as in vivo implantable devices in patients, particularly in mammals and especially in humans.

Elastomeric Matrix Physical Properties

Elastomeric matrix 10 can have any suitable bulk density, also known as specific gravity, consistent with its other properties. For example, in one embodiment, the bulk density, as measured pursuant to the test method described in ASTM Standard D3574, may be from about 0.005 g/cc to about 0.15 g/cc (from about 0.31 lb/ft³ to about 9.4 lb/ft³). In another embodiment, the bulk density may be from about 0.008 g/cc to about 0.127 g/cc (from about 0.5 lb/ft³ to about 8 lb/ft³). In another embodiment, the bulk density may be from about 0.015 g/cc to about 0.115 g/cc (from about 0.93 lb/ft³ to about 7.2 lb/ft³). In another embodiment, the bulk density may be from about 0.024 g/cc to about 0.104 g/cc (from about 1.5 lb/ft³ to about 6.5 lb/ft³).

Elastomeric matrix 10 can have any suitable microscopic surface area consistent with its other properties. Those skilled in the art, e.g., from an exposed plane of the porous material, can routinely estimate the microscopic surface area from the pore frequency, e.g., the number of pores per linear millimeter, and can routinely estimate the pore frequency from the average cell side diameter in μm.

Other suitable physical properties will be apparent to, or will become apparent to, those skilled in the art.

Elastomeric Matrix Mechanical Properties

In one embodiment, reticulated elastomeric matrix 10 has sufficient structural integrity to be self-supporting and free-standing in vitro. However, in another embodiment, elastomeric matrix 10 can be funished with structural supports such as ribs or struts.

The reticulated elastomeric matrix 10 has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix 10.

Thus, for example, in one embodiment reticulated elastomeric matrix 10 may have a tensile strength of from about 700 kg/m² to about 52,500 kg/m² (from about 1 psi to about 75 psi). In another embodiment, elastomeric matrix 10 may have a tensile strength of from about 700 kg/m² to about 21,000 kg/m² (from about 1 psi to about 30 psi).

Sufficient ultimate tensile elongation is also desirable. For example, in another embodiment, reticulated elastomeric matrix 10 has an ultimate tensile elongation of at least about 150%. In another embodiment, elastomeric matrix 10 has an ultimate tensile elongation of at least about 200%. In another embodiment, elastomeric matrix 10 has an ultimate tensile elongation of at least about 500%.

One embodiment for use in the practice of the invention is a reticulated elastomeric matrix 10 which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration, in situ. Furthermore, in another embodiment, an elastomeric matrix has the herein described resilient-compressibility after being compressed about 5-95% of an original dimension (e.g., compressed about 19/20th-1/20th of an original dimension). In another embodiment, an elastomeric matrix has the herein described resilient-compressibility after being compressed about 10-90% of an original dimension (e.g., compressed about 9/10th-1/10th of an original dimension). As used herein, elastomeric matrix 10 has “resilient-compressibility”, i.e., is “resiliently-compressible”, when the second, working configuration, in vitro, is at least about 50% of the size of the relaxed configuration in at least one dimension. In another embodiment, the resilient-compressibility of elastomeric matrix 10 is such that the second, working configuration, in vitro, is at least about 80% of the size of the relaxed configuration in at least one dimension. In another embodiment, the resilient-compressibility of elastomeric matrix 10 is such that the second, working configuration, in vitro, is at least about 90% of the size of the relaxed configuration in at least one dimension. In another embodiment, the resilient-compressibility of elastomeric matrix 10 is such that the second, working configuration, in vitro, is at least about 97% of the size of the relaxed configuration in at least one dimension.

In another embodiment, an elastomeric matrix has the herein described resilient-compressibility after being compressed about 5-95% of its original volume (e.g., compressed about 19/20th-1/20th of its original volume). In another embodiment, an elastomeric matrix has the herein described resilient-compressibility after being compressed about 10-90% of its original volume (e.g., compressed about 9/10th-1/10th of its original volume). As used herein, “volume” is the volume swept-out by the outermost 3-dimensional contour of the elastomeric matrix. In another embodiment, the resilient-compressibility of elastomeric matrix 10 is such that the second, working configuration, in vivo, is at least about 50% of the volume occupied by the relaxed configuration. In another embodiment, the resilient-compressibility of elastomeric matrix 10 is such that the second, working configuration, in vivo, is at least about 80% of the volume occupied by the relaxed configuration. In another embodiment, the resilient-compressibility of elastomeric matrix 10 is such that the second, working configuration, in vivo, is at least about 90% of the volume occupied by the relaxed configuration. In another embodiment, the resilient-compressibility of elastomeric matrix 10 is such that the second, working configuration, in vivo, is at least about 97% of the of the volume occupied by the relaxed configuration. In another embodiment, elastomeric matrix 10 can be inserted by an open surgical procedure.

In one embodiment, reticulated elastomeric matrix 10 has a compressive strength of from about 700 to about 140,000 kg/m² (from about 1 to about 200 psi) at 50% compression strain. In another embodiment, reticulated elastomeric matrix 10 has a compressive strength of from about 700 to about 35,000 kg/m² (from about 1 to about 50 psi) at 50% compression strain. In another embodiment, reticulated elastomeric matrix 10 has a compressive strength of from about 700 to about 21,000 kg/m² (from about 1 to about 30 psi) at 50% compression strain. In another embodiment, reticulated elastomeric matrix 10 has a compressive strength of from about 7,000 to about 210,000 kg/m² (from about 10 to about 300 psi) at 75% compression strain. In another embodiment, reticulated elastomeric matrix 10 has a compressive strength of from about 7,000 to about 70,000 kg/m² (from about 10 to about 100 psi) at 75% compression strain. In another embodiment, reticulated elastomeric matrix 10 has a compressive strength of from about 7,000 to about 28,000 kg/m² (from about 10 to about 40 psi) at 75% compression strain.

In another embodiment, reticulated elastomeric matrix 10 has a compression set, when compressed to 50% of its thickness at about 25° C., i.e., pursuant to ASTM D3574, of not more than about 30%. In another embodiment, elastomeric matrix 10 has a compression set of not more than about 20%. In another embodiment, elastomeric matrix 10 has a compression set of not more than about 10%. In another embodiment, elastomeric matrix 10 has a compression set of not more than about 5%.

In another embodiment, reticulated elastomeric matrix 10 has a tear strength, as measured pursuant to the test method described in ASTM Standard D3574, of from about 0.18 to about 1.78 kg/linear cm (from about 1 to about 10 lbs/linear inch).

Table 1 summarizes mechanical property and other properties applicable to embodiments of reticulated elastomeric matrix 10. Additional suitable mechanical properties will be apparent to, or will become apparent to, those skilled in the art.

TABLE 1
Properties of Reticulated Elastomeric Matrix 10

| | Typical | Exemplary |
|---|---|---|
| Property | Values | Test Procedure |
| Specific Gravity/Bulk Density (lb/ft³) | 0.31-9.4 | ASTM D3574 |
| Tensile Strength (psi) | 1-75 | ASTM D3574 |
| Ultimate Tensile Elongation (%) | ≧150 | ASTM D3574 |
| Compressive Strength at 50% | 1-200 | ASTM D3574 |
| Compression (psi) | | |
| Compressive Strength at 75% | 10-300 | ASTM D3574 |
| Compression (psi) | | |
| 25% Compression Set, 22 hours | ≦30 | ASTM D3574 |
| at 25° C. (%) | | |
| 50% Compression Set, 22 hours | ≦15 | ASTM D3574 |
| at 25° C. (%) | | |
| Tear Strength (lbs/linear inch) | 1-10 | ASTM D3574 |

The mechanical properties of the porous materials described herein, if not indicated otherwise, may be determined according to ASTM D3574-01 entitled “Standard Test Methods for Flexible Cellular Materials—Slab, Bonded and Molded Urethane Foams”, or other such method as is known to be appropriate by those skilled in the art.

Furthermore, if porosity is to be imparted to the elastomer employed for elastomeric matrix 10 after rather than during the polymerization reaction, good processability is also desirable for post-polymerization shaping and fabrication. For example, in one embodiment, elastomeric matrix 10 has low tackiness.

Biodurability and Biocompatibility

In one embodiment, elastomers are sufficiently biodurable so as to be suitable for long-term implantation in patients, e.g., animals or humans. Biodurable elastomers and elastomeric matrices have chemical, physical and/or biological properties so as to provide a reasonable expectation of biodurability, meaning that the elastomers will continue to exhibit stability when implanted in an animal, e.g., a mammal, for a period of at least 29 days. The intended period of long term implantation may vary according to the particular application. For many applications, substantially longer periods of implantation may be required and for such applications biodurability for periods of at least 6, 12 or 24 months, or as much as 5 years, may be desirable. Of especial benefit are elastomers that may be considered biodurable for the life of a patient. In the case of the possible use of an embodiment of elastomeric matrix 10 to treat cranial aneurysms, because such conditions may present themselves in rather young human patients, perhaps in their thirties, biodurability in excess of 50 years may be advantageous.

In another embodiment, the period of implantation will be at least sufficient for cellular ingrowth and proliferation to commence, for example, in at least about 4-8 weeks. In another embodiment, elastomers are sufficiently well characterized to be suitable for long-term implantation by having been shown to have such chemical, physical and/or biological properties as to provide a reasonable expectation of biodurability, meaning that the elastomers will continue to exhibit biodurability when implanted for extended periods of time.

Without being bound by any particular theory, biodurability of the elastomeric matrix of the invention can be promoted by selecting a biodurable polymer(s) as the polymeric component of the flowable material used in the sacrificial molding or lyophilization processes for preparing a reticulated elastomeric matrix of the invention. Furthermore, additional considerations to promote the biodurability of the elastomeric matrix formed by a process comprising polymerization, crosslinking, foaming and reticulation include the selection of starting components that are biodurable and the stoichiometric ratios of those components, such that the elastomeric matrix retains the biodurability of its components. For example, elastomeric matrix biodurability can be promoted by minimizing the presence and formation of chemical bonds and groups, such as ester groups, that are susceptible to hydrolysis, e.g., at the patient's body fluid temperature and pH. As a further example, a curing step in excess of about 2 hours can be performed after crosslinking and foaming to minimize the presence of free amine groups in the elastomeric matrix. Moreover, it is important to minimize degradation that can occur during the elastomeric matrix preparation process, e.g., because of exposure to shearing or thermal energy such as may occur during admixing, dissolution, crosslinking and/or foaming, by ways known to those in the art.

As previously discussed, biodurable elastomers and elastomeric matrices are stable for extended periods of time in a biological environment. Such products do not exhibit significant symptoms of breakdown, degradation, erosion or significant deterioration of mechanical properties relevant to their use when exposed to biological environments and/or bodily stresses for periods of time commensurate with that use. However, some amount of cracking, fissuring or a loss in toughness and stiffening—at times referred to as ESC or environmental stress cracking—may not be relevant to endovascular and other uses as described herein. Many in vivo applications, e.g., when elastomeric matrix 10 is used for treatment of vascular abnormalities, expose it to little, if any, mechanical stress and, thus, are unlikely to result in mechanical failure leading to serious patient consequences. Accordingly, the absence of ESC may not be a prerequisite for biodurability of suitable elastomers in such applications for which the present invention is intended because elastomeric properties become less important as endothielozation, encapsulation and cellular ingrowth and proliferation advance.

Furthermore, in certain implantation applications, it is anticipated that elastomeric matrix 10 will become in the course of time, for example, in 2 weeks to 1 year, walled-off or encapsulated by tissue, scar tissue or the like, or incorporated and totally integrated into, e.g., the tissue being repaired or the lumen being treated. In this condition, elastomeric matrix 10 has reduced exposure to mobile or circulating biological fluids. Accordingly, the probabilities of biochemical degradation or release of undesired, possibly nocuous, products into the host organism may be attenuated if not eliminated.

In one embodiment, the elastomeric matrix has good biodurability accompanied by good biocompatibility such that the elastomer induces few, if any, adverse reactions in vivo. To that end, in another embodiment for use in the invention are elastomers or other materials that are free of biologically undesirable or hazardous substances or structures that can induce such adverse reactions or effects in vivo when lodged in an intended site of implantation for the intended period of implantation. Such elastomers accordingly should either entirely lack or should contain only very low, biologically tolerable quantities of cytotoxins, mutagens, carcinogens and/or teratogens. In another embodiment, biological characteristics for biodurability of elastomers to be used for fabrication of elastomeric matrix 10 include at least one of resistance to biological degradation, and absence of or extremely low: cytotoxicity, hemotoxicity, carcinogenicity, mutagenicity, or teratogenicity.

Process Aspects of the Invention

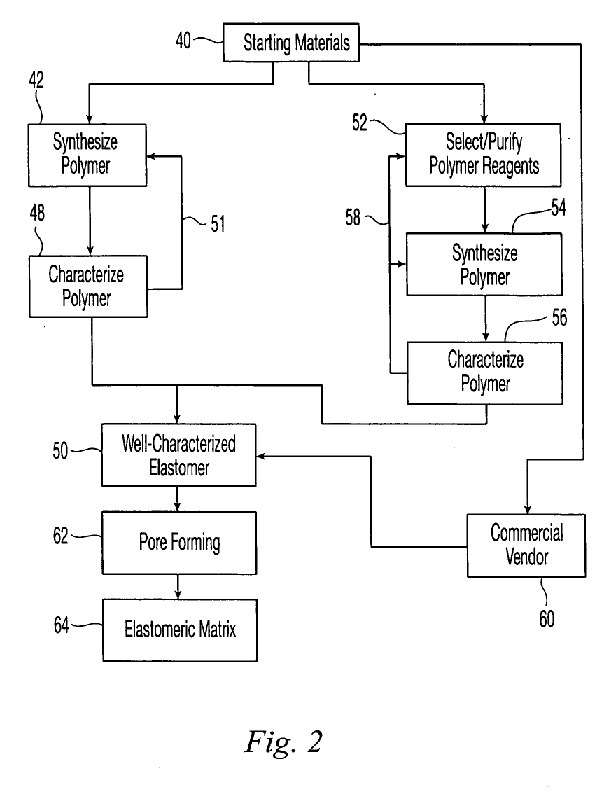

Referring now to FIG. 2, the schematic block flow diagram shown gives a broad overview of a process according to the invention whereby an implantable device comprising a biodurable, porous, reticulated, elastomeric matrix 10 can be prepared from raw elastomer or elastomer reagents by one or another of several different process routes.

In a first route, elastomers prepared by a process according to the invention, as described herein, are rendered to comprise a plurality of cells by using, e.g., a blowing agent or agents, employed during their preparation. In particular, starting materials 40, which may comprise, for example, a polyol component, an isocyanate, optionally a crosslinker, and any desired additives such as surfactants and the like, are employed to synthesize the desired elastomeric polymer, polymerization step 42 either with or without significant foaming or other pore-generating activity. The starting materials are selected to provide desirable mechanical properties and to enhance biocompatibility and biodurability.

The elastomeric polymer product of step 42 is then characterized, in step 48, as to chemical nature and purity, physical and mechanical properties and, optionally, also as to biological characteristics, all as described above, yielding well-characterized elastomer 50. Optionally, the characterization data can be employed to control or modify step 42 to enhance the process or the product, as indicated by forked arrow 51. Selecting elastomer 50 to be solvent-soluble, for example by ensuring that it is not crosslinked, enables elastomer 50 to be closely analyzed for effective process control and product characterization.

Alternatively, in a second route, the elastomeric polymer reagents employed in starting material 40 may be selected to avoid adverse by-products or residuals and purified, if necessary, step 52. Polymer synthesis, step 54, is then conducted on the selected and purified starting materials and is conducted to avoid generation of adverse by-products or residuals. The elastomeric polymer produced in step 54 is then characterized, step 56, as described for step 48, to facilitate production of a high quality, well-defined product, well-characterized elastomer 50. In another embodiment, the characterization results are fed back for process control as indicated by forked arrow 58, to facilitate production of a high quality, well-defined product, well-characterized elastomer 50.

Pursuant to a third route, well-characterized elastomer 50 is generated from starting materials 40 and supplied to the process facility by a commercial vendor 60. Such elastomers are synthesized pursuant to known methods and subsequently rendered porous. An exemplary elastomer of this type is BIONATE® 80A polyurethane elastomer. The elastomer 50 can be rendered porous, e.g., by a blowing agent employed in a polymerization reaction or in a post-polymerization step.

The invention provides, in one embodiment, a reticulated biodurable elastomeric matrix comprising polymeric elements which are specifically designed for the purpose of biomedical implantation. It comprises biodurable polymeric materials and is prepared by a process or processes which avoid chemically changing the polymer, the formation of undesirable by-products, and residuals comprising undesirable unreacted starting materials. In some cases, foams comprising polyurethanes and created by known techniques may not be appropriate for long-term endovascular, orthopedic and related applications because of, e.g., the presence of undesirable unreacted starting materials or undesirable by-products.

In one embodiment, well-characterized elastomer 50 is thermoplastic with a Vicat softening temperature below about 120° C. and has a molecular weight facilitating solvent or melt processing. In another embodiment, well-characterized elastomer 50 is thermoplastic with a Vicat softening temperature below about 100° C. and has a molecular weight facilitating solvent or melt processing. Elastomer 50 can conveniently be furnished in divided form at this stage, e.g., as pellets, to facilitate subsequent processing.

Well-characterized elastomer 50 is rendered porous in a pore forming step, step 62, yielding porous elastomer 64. In one embodiment, step 62 employs a process which leaves no undesirable residuals, such as residuals adverse to biodurability, and does not change the chemistry of the elastomer 50. In another embodiment, porous biodurable elastomer 64 can be washed with solvent, for example a volatile organic such as hexane or isopropanol, and air dried. Fabrication step 62 may include a more or less complex molding step or feature, for example to provide bulk stock in the form of a strip, roll, block or the like of porous biodurable elastomer 64.

Porous biodurable elastomer 64 may be used to manufacture elastomeric matrix 10, for example by cutting to a desired shape and size, if necessary.

In another embodiment, chemical characteristics for biodurability of elastomers to be used for fabrication of elastomeric matrix 10 include one or more of: good oxidative stability; a chemistry that is free or substantially free, of linkages that are prone to biological degradation, for example polyether linkages or hydrolyzable ester linkages that may be introduced by incorporating a polyether or polyester polyol component into the polyurethane; a chemically well-defined product which is relatively refined or purified and free, or substantially free, of adverse impurities, reactants, by-products; oligomers and the like; a well-defined molecular weight, unless the elastomer is crosslinked; and solubility in a biocompatible solvent unless, of course, the elastomer is crosslinked.

In another embodiment, process-related characteristics, referring to a process used for the preparation of the elastomer of the solid phase 12, for biodurability of elastomers to be used for fabrication of elastomeric matrix 10 include one or more of: process reproducibility; process control for product consistency; and avoidance or substantial removal of adverse impurities, reactants, by-products, oligomers and the like.

The pore-making, reticulation and other post-polymerization processes of the invention, discussed below, are, in certain embodiments, carefully designed and controlled to avoid changing the chemistry of the polymer. To this end, in certain embodiments, processes of the invention avoid introducing undesirable residuals or otherwise adversely affecting the desirable biodurability properties of the starting material(s). In another embodiment, the starting material(s) may be further processed and/or characterized to enhance, provide or document a property relevant to biodurability. In another embodiment, the requisite properties of elastomers can be characterized as appropriate and the process features can be adapted or controlled to enhance biodurability, pursuant to the teachings of the present specification.

Elastomeric Matrices from Elastomer Polymerization, Crosslinking and Foaming

In further embodiments, the invention provides a porous biodurable elastomer and a process for polymerizing, crosslinking and foaming the same which can be used to produce a biodurable reticulated elastomeric matrix as described herein. In another embodiment, reticulation follows.

More particularly, in another embodiment, the invention provides a process for preparing a biodurable elastomeric polyurethane matrix which comprises synthesizing the matrix from a polycarbonate polyol component and an isocyanate component by polymerization, crosslinking and foaming, thereby forming pores, followed by reticulation of the foam to provide a reticulated product. The product is designated as a polycarbonate polyurethane, being a polymer comprising urethane groups formed from, e.g., the hydroxyl groups of the polycarbonate polyol component and the isocyanate groups of the isocyanate component. In this embodiment, the process employs controlled chemistry to provide a reticulated elastomer product with good biodurability characteristics. Pursuant to the invention, the polymerization is conducted to provide a foam product employing chemistry that avoids biologically undesirable or nocuous constituents therein.

In one embodiment, as one starting material, the process employs at least one polyol component. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. Exemplary polyols can comprise, on the average, from about 2 to about 5 hydroxyl groups per molecule. In one embodiment, as one starting material, the process employs a difunctional polyol component. In this embodiment, because the hydroxyl group functionality of the diol is about 2, it does not provide the so-called “soft segment” with soft segment crosslinking. In another embodiment, as one starting material of the polyol component, the process employs a multi-functional polyol component in sufficient quantity to provide a controlled degree of soft segment crosslinking. In another embodiment, the process provides sufficient soft segment crosslinking to yield a stable foam. In another embodiment, the soft segment is composed of a polyol component that is generally of a relatively low molecular weight, typically from about 1,000 to about 6,000 Daltons. Thus, these polyols are generally liquids or low-melting-point solids. This soft segment polyol is terminated with hydroxyl groups, either primary or secondary. In another embodiment, a soft segment polyol component has about 2 hydroxyl groups per molecule. In another embodiment, a soft segment polyol component has greater than about 2 hydroxyl groups per molecule; more than 2 hydroxyl groups per polyol molecule are required of some polyol molecules to impart soft-segment crosslinking.

In one embodiment, the average number of hydroxyl groups per molecule in the polyol component is about 2. In another embodiment, the average number of hydroxyl groups per molecule in the polyol component is greater than about 2. In another embodiment, the average number of hydroxyl groups per molecule in the polyol component is greater than 2. In one embodiment, the polyol component comprises a tertiary carbon linkage. In one embodiment, the polyol component comprises a plurality of tertiary carbon linkages.

In one embodiment, the polyol component is a polyether polyol, polyester polyol, polycarbonate polyol, hydrocarbon polyol, polysiloxane polyol, poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol, poly(hydrocarbon-co-siloxane) polyol, or mixtures thereof.

Polyether-type polyols are oligomers of, e.g., alkylene oxides such as ethylene oxide or propylene oxide, polymerized with glycols or polyhydric alcohols, the latter to result in hydroxyl functionalities greater than 2 to allow for soft segment crosslinking. Polyester-type polyols are oligomers of, e.g., the reaction product of a carboxylic acid with a glycol or triol, such as ethylene glycol adipate, propylene glycol adipate, butylene glycol adipate, diethylene glycol adipate, phthalates, polycaprolactone and castor oil. When the reactants include those with hydroxyl functionalities greater than 2, e.g., polyhydric alcohols, soft segment crosslinking is possible.

Polycarbonate-type polyols are biodurable and typically result from the reaction, with a carbonate monomer, of one type of hydrocarbon diol or, for a plurality of diols, hydrocarbon diols each with a different hydrocarbon chain length between the hydroxyl groups. The length of the hydrocarbon chain between adjacent carbonates is the same as the hydrocarbon chain length of the original diol(s). For example, a difunctional polycarbonate polyol can be made by reacting 1,6-hexanediol with a carbonate, such as sodium hydrogen carbonate, to provide the polycarbonate-type polyol 1,6-hexanediol carbonate. The molecular weight for the commercial-available products of this reaction varies from about 1,000 to about 5,000 Daltons. If the polycarbonate polyol is a solid at 25° C., it is typically melted prior to further processing. Alternatively, in one embodiment, a liquid polycarbonate polyol component can prepared from a mixture of hydrocarbon diols, e.g., all three or any binary combination of 1,6-hexanediol, cyclohexyl dimethanol and 1,4-butanediol. Without being bound by any particular theory, such a mixture of hydrocarbon diols is thought to break-up the crystallinity of the product polycarbonate polyol component, rendering it a liquid at 25° C., and thereby, in foams comprising it, yield a relatively softer foam.

When the reactants used to produce the polycarbonate polyol include those with hydroxyl functionalities greater than 2, e.g., polyhydric alcohols, soft segment crosslinking is possible. Polycarbonate polyols with an average number of hydroxyl groups per molecule greater than 2, e.g., a polycarbonate triol, can be made by using, for example, hexane triol, in the preparation of the polycarbonate polyol component. To make a liquid polycarbonate triol component, mixtures with other hydroxyl-comprising materials, for example, cyclohexyl trimethanol and/or butanetriol, can be reacted with the carbonate along with the hexane triol.

Commercial hydrocarbon-type polyols typically result from the free-radical polymerization of dienes with vinyl monomers, therefore, they are typically difunctional hydroxyl-terminated materials.

Polysiloxane polyols are oligomers of, e.g., alkyl and/or aryl substituted siloxanes such as dimethyl siloxane, diphenyl siloxane or methyl phenyl siloxane, comprising hydroxyl end-groups. Polysiloxane polyols with an average number of hydroxyl groups per molecule greater than 2, e.g., a polysiloxane triol, can be made by using, for example, methyl hydroxymethyl siloxane, in the preparation of the polysiloxane polyol component.

A particular type of polyol need not, of course, be limited to those formed from a single monomeric unit. For example, a polyether-type polyol can be formed from a mixture of ethylene oxide and propylene oxide.

Additionally, in another embodiment, copolymers or copolyols can be formed from any of the above polyols by methods known to those in the art. Thus, the following binary component polyol copolymers can be used: poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol and poly(hydrocarbon-co-siloxane) polyol. For example, a poly(ether-co-ester) polyol can be formed from units of polyethers formed from ethylene oxide copolymerized with units of polyester comprising ethylene glycol adipate. In another embodiment, the copolymer is a poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol, poly(carbonate-co-siloxane) polyol, poly(hydrocarbon-co-siloxane) polyol or mixtures thereof. In another embodiment, the copolymer is a poly(carbonate-co-hydrocarbon) polyol. For example, a poly(carbonate-co-hydrocarbon) polyol can be formed by polymerizing 1,6-hexanediol, 1,4-butanediol and a hydrocarbon-type polyol with carbonate.

In another embodiment, the polyol component is a polyether polyol, polycarbonate polyol, hydrocarbon polyol, polysiloxane polyol, poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or mixtures thereof. In another embodiment, the polyol component is a polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol or mixtures thereof. In another embodiment, the polyol component is a polycarbonate polyol, poly(carbonate-co-hydrocarbon) polyol, poly(carbonate-co-siloxane) polyol, poly(hydrocarbon-co-siloxane) polyol or mixtures thereof. In another embodiment, the polyol component is a polycarbonate polyol, poly(carbonate-co-hydrocarbon) polyol, poly(carbonate-co-siloxane) polyol or mixtures thereof. In another embodiment, the polyol component is a polycarbonate polyol.

Furthermore, in another embodiment, mixtures, admixtures and/or blends of polyols and copolyols can be used in the elastomeric matrix of the present invention. In another embodiment, the molecular weight of the polyol is varied. In another embodiment, the functionality of the polyol is varied.

In another embodiment, as either difunctional polycarbonate polyols or difunctional hydrocarbon polyols cannot, on their own, induce soft segment crosslinking, higher functionality is introduced into the formulation through the use of a chain extender component with a hydroxyl group functionality greater than about 2. In another embodiment, higher functionality is introduced through the use of an isocyanate component with an isocyanate group functionality greater than about 2.

Commercial polycarbonate diols with molecular weights of from about 2,000 to about 6,000 Daltons are available from Stahl, Inc. (Netherlands) and Bayer Corp. (Leverkusen, Germany). Commercial hydrocarbon polyols are available from Sartomer (Exton, Pa.). Commercial polyether polyols are readily available, such as the PLURACOL®, e.g., PLURACOL® GP430 with functionality of 3 and LUPRANOL® lines from BASF Corp. (Wyandotte, Mich.), VORANOL® from Dow Chemical Corp. (Midland, Mich.), BAYCOLL® B, DESMOPHEN® and MULTRANOL® from Bayer, and from Huntsman Corp. (Madison Heights, Mich.). Commercial polyester polyols are readily available, such as LUPRAPHEN® from BASF, TONE® polycaprolactone and VORANOL from Dow, BAYCOLL A and the DESMOPHEN® U series from Bayer, and from Huntsman. Commercial polysiloxane polyols are readily available, such as from Dow.

The process also employs at least one isocyanate component and, optionally, at least one chain extender component to provide the so-called “hard segment”. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water. In particular, when water is present, e.g., as the blowing agent or a component thereof, the water can react with an isocyanate group of the isocyanate component to form an amine, which can react with another isocyanate group to form a urea moiety. Thus, the final polymer is a polyurethane-urea because it can contain urethane moieties and urea moieties. For the purposes of this is application, a “polyurethane” formed from an isocyanate component includes a polyurethane, a polyurethane-urea, and their mixtures. In one embodiment, a polyurethane of the invention formed from an isocyanate component using water as a blowing agent comprises, on average, more urethane moieties than urea moieties.

In one embodiment, the average number of isocyanate groups per molecule in the isocyanate component is about 2. In another embodiment, the average number of isocyanate groups per molecule in the isocyanate component is greater than about 2. In another embodiment, the average number of isocyanate groups per molecule in the isocyanate component is greater than 2. In another embodiment, the average number of isocyanate groups per molecule in the isocyanate component is greater than 2.05. In another embodiment, the average number of isocyanate groups per molecule in the isocyanate component is greater than about 2.05. In another embodiment, the average number of isocyanate groups per molecule in the isocyanate component is greater than 2.1. In another embodiment, the average number of isocyanate groups per molecule in the isocyanate component is greater than about 2.1. In another embodiment, the average number of isocyanate groups per molecule in the isocyanate component is greater than 2.2. In another embodiment, the average number of isocyanate groups per molecule in the isocyanate component is greater than about 2.2.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to 1.029. In another embodiment, the isocyanate index is from about 0.9 to 1.028. In another embodiment, the isocyanate index is from about 0.9 to about 1.025. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H₁₂ MDI”), and mixtures thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), 2,4-toluene diisocyanate (“2,4-TDI”), 2,6-toluene diisocyanate(“2,6-TDI”), m-tetramethylxylene diisocyanate, and mixtures thereof.

Exemplary isocyanate components comprising, on the average, greater than about 2 isocyanate groups per molecule, include an adduct of hexamethylene diisocyanate and water comprising about 3 isocyanate groups, available commercially as DESMODUR® N100 from Bayer, and a trimer of hexamethylene diisocyanate comprising about 3 isocyanate groups, available commercially as MONDUR® N3390 from Bayer.

In one embodiment, the isocyanate component contains a mixture of at least about 5% by weight of 2,4′-MDI with the balance 4,4′-MDI, thereby excluding the polyether or polycarbonate polyurethanes having less than 3% by weight of 2,4′-MDI disclosed by Brady '550. In another embodiment, the isocyanate component contains a mixture of at least 5% by weight of 2,4′-MDI with the balance 4,4′-MDI. In another embodiment, the isocyanate component contains a mixture of from about 5% to about 50% by weight of 2,4′-MDI with the balance 4,4′-MDI. In another embodiment, the isocyanate component contains a mixture of from 5% to about 50% by weight of 2,4′-MDI with the balance 4,4′-MDI. In another embodiment, the isocyanate component contains a mixture of from about 5% to about 40% by weight of 2,4′-MDI with the balance 4,4′-MDI. In another embodiment, the isocyanate component contains a mixture of from 5% to about 40% by weight of 2,4′-MDI with the balance 4,4′-MDI. In another embodiment, the isocyanate component contains a mixture of from 5% to about 35% by weight of 2,4′-MDI with the balance 4,4′-MDI. Without being bound by any particular theory, it is thought that the use of higher amounts of 2,4′-MDI in a blend with 4,4′-MDI results in a softer elastomeric matrix because of the disruption of the crystallinity of the hard segment arising out of the asymmetric 2,4′-MDI structure.

Suitable diisocyanates include MDI, such as ISONATE® 125M, certain members of the PAPI® series from Dow and MONDUR M from Bayer; isocyanates containing a mixture of 4,4′-MDI and 2,4′-MDI, such as RUBINATE® 9433 and RUBINATE 9258, each from Huntsman, and ISONATE 50 OP from Dow; TDI, e.g., from Lyondell Corp. (Houston, Tex.); isophorone diisocyanate, such as VESTAMAT® from Degussa (Germany); H₁₂ MDI, such as DESMODUR W from Bayer; and various diisocyanates from BASF.

Suitable isocyanate components comprising, on the average, greater than about 2 isocyanate groups per molecule, include the following modified diphenylmethane-diisocyanate type, each available from Dow: ISOBIND® 1088, with an isocyanate group functionality of about 3; ISONATE 143L, with an isocyanate group functionality of about 2.1; PAPI 27, with an isocyanate group functionality of about 2.7; PAPI 94, with an isocyanate group functionality of about 2.3; PAPI 580N, with an isocyanate group functionality of about 3; and PAPI 20, with an isocyanate group functionality of about 3.2. Other isocyanate components comprising, on the average, greater than about 2 isocyanate groups per molecule, include the following, each available from Huntsman: RUBINATE® 9433, with an isocyanate group functionality of about 2.01; and RUBINATE 9258, with an isocyanate group functionality of about 2.33.

Exemplary chain extenders include diols, diamines, alkanol amines and mixtures thereof. In one embodiment, the chain extender is an aliphatic diol having from 2 to 10 carbon atoms. In another embodiment, the diol chain extender is selected from ethylene glycol, 1,2-propane diol, 1,3-propane diol, 1,4-butane diol, 1,5-pentane diol, diethylene glycol, triethylene glycol and mixtures thereof. In another embodiment, the chain extender is a diamine having from 2 to 10 carbon atoms. In another embodiment, the diamine chain extender is selected from ethylene diamine, 1,3-diaminobutane, 1,4-diaminobutane, 1,5 diaminopentane, 1,6-diaminohexane, 1,7-diaminoheptane, 1,8-diaminooctane, isophorone diamine and mixtures thereof In another embodiment, the chain extender is an alkanol amine having from 2 to 10 carbon atoms. In another embodiment, the alkanol amine chain extender is selected from diethanolamine, triethanolamine, isopropanolamine, dimethylethanolamine, methyldiethanolamine, diethylethanolamine and mixtures thereof.

Commercially available chain extenders include the the JEFFAMINE® series of diamines, triamines and polyetheramines available from Huntsman, VERSAMIN® isophorone diamine from Creanova, the VERSALINK® series of diamines available from Air Products Corp. (Allentown, Pa.), ethanolamine, diethylethanolamine and isopropanolamine available from Dow, and various chain extenders from Bayer, BASF and UOP Corp. (Des Plaines, Ill.).

In one embodiment, a small quantity of an optional ingredient, such as a multi-functional hydroxyl compound or other crosslinker having a functionality greater than 2, e.g., glycerol, is present to allow crosslinking. In another embodiment, the optional multi-functional crosslinker is present in an amount just sufficient to achieve a stable foam, i.e., a foam that does not collapse to become non-foamlike. Alternatively, or in addition, polyfunctional adducts of aliphatic and cycloaliphatic isocyanates can be used to impart crosslinking in combination with aromatic diisocyanates. Alternatively, or in addition, polyfunctional adducts of aliphatic and cycloaliphatic isocyanates can be used to impart crosslinking in combination with aliphatic diisocyanates.

Optionally, the process employs at least one catalyst in certain embodiments selected from a blowing catalyst, e.g., a tertiary amine, a gelling catalyst, e.g., dibutyltin dilaurate, and mixtures thereof. Moreover, it is known in the art that tertiary amine catalysts can also have gelling effects, that is, they can act as a blowing and gelling catalyst. Exemplary tertiary amine catalysts include the TOTYCAT® line from Toyo Soda Co. (Japan), the TEXACAT® line from Texaco Chemical Co. (Austin, Tex.), the KOSMOS® and TEGO® lines from Th. Goldschmidt Co. (Germany), the DMP® line from Rohm and Haas (Philadelphia, Pa.), the KAO LIZER® line from Kao Corp. (Japan), and the QUINCAT® line from Enterprise Chemical Co. (Altamonte Springs, Fla.). Exemplary organotin catalysts include the FOMREZ® and FOMREZ UL®D lines from Witco Corporation (Middlebury, Conn.), the COCURE® and COSCAT® lines from Cosan Chemical Co. (Carlstadt, N.J.), and the DABCO® and POLYCAT® lines from Air Products.

In certain embodiments, the process employs at least one surfactant. Exemplary surfactants include DC 5241 from Dow Coming (Midland, Mich.) and other non-ionic organosilicones, such as the polydimethylsiloxane types available from Dow Coming, Air Products and General Electric (Waterford, N.Y.).

Crosslinked polyurethanes may be prepared by approaches which include the prepolymer process and the one-shot process. An embodiment involving a prepolymer is as follows. First, the prepolymer is prepared by a conventional method from at least one isocyanate component (e.g., MDI) and at least one multi-functional soft segment material with a functionality greater than 2 (e.g., a polyether-based soft segment with a functionality of 3). Then, the prepolymer, optionally at least one catalyst (e.g., dibutyltin dilaurate) and at least one difunctional chain extender (e.g., 1,4-butanediol) are admixed in a mixing vessel to cure or crosslink the mixture. In another embodiment, crosslinking takes place in a mold. In another embodiment, crosslinking and foaming, i.e., pore formation, take place together. In another embodiment, crosslinking and foaming take place together in a mold.

Alternatively, the so-called “one-shot” approach may be used. A one-shot embodiment requires no separate prepolymer-making step. In one embodiment, the starting materials, such as those described in the previous paragraph, are admixed in a mixing vessel and then foamed and crosslinked. In another embodiment, the ingredients are heated before they are admixed. In another embodiment, the ingredients are heated as they are admixed. In another embodiment, crosslinking takes place in a mold. In another embodiment, foaming and crosslinking take place together. In another embodiment, crosslinking and foaming take place together in a mold. In another embodiment, all of the ingredients except for the isocyanate component are admixed in a mixing vessel. The isocyanate component is then added, e.g., with high-speed stirring, and crosslinking and foaming ensue. In another embodiment, this foaming mix is poured into a mold and allowed to rise.

In another embodiment, the polyol component is admixed with the isocyanate component and other optional additives, such as a viscosity modifier, surfactant and/or cell opener, to form a first liquid. In another embodiment, the polyol component is a liquid at the admixing temperature or over the admixing temperature range. In another embodiment, the polyol component is a solid, therefore, the polyol component is liquefied prior to admixing, e.g., by heating. In another embodiment, the polyol component is a solid, therefore, the admixing temperature or admixing temperature range is raised such that the polyol component is liquefied prior to admixing. Next, a second liquid is formed by admixing a blowing agent and optional additives, such as gelling catalyst and/or blowing catalyst. Then, the first liquid and the second liquid are admixed in an admixing vessel and then foamed and crosslinked.

In one embodiment, the invention provides a process for preparing a flexible polyurethane biodurable matrix capable of being reticulated based on polycarbonate polyol component and isocyanate component starting materials. In another embodiment, a porous biodurable elastomer polymerization process for making a resilient polyurethane matrix is provided which process comprises admixing a polycarbonate polyol component and an aliphatic isocyanate component, for example H₁₂ MDI.

In another embodiment, the foam is substantially free of isocyanurate linkages, thereby excluding the polyether or polycarbonate polyurethanes having isocyanurate linkages disclosed by Brady '550. In another embodiment, the foam has no isocyanurate linkages. In another embodiment, the foam is substantially free of biuret linkages. In another embodiment, the foam has no biuret linkages. In another embodiment, the foam is substantially free of allophanate linkages. In another embodiment, the foam has no allophanate linkages. In another embodiment, the foam is substantially free of isocyanurate and biuret linkages. In another embodiment, the foam has no isocyanurate and biuret linkages. In another embodiment, the foam is substantially free of isocyanurate and allophanate linkages. In another embodiment, the foam has no isocyanurate and allophanate linkages. In another embodiment, the foam is substantially free of allophanate and biuret linkages. In another embodiment, the foam has no allophanate and biuret linkages. In another embodiment, the foam is substantially free of allophanate, biuret and isocyanurate linkages. In another embodiment, the foam has no allophanate, biuret and isocyanurate linkages. Without being bound by any particular theory, it is thought that the absence of allophanate, biuret and/or isocyanurate linkages provides an enhanced degree of flexibility to the elastomeric matrix because of lower crosslinking of the hard segments.

In certain embodiments, additives helpful in achieving a stable foam, for example, surfactants and catalysts, can be included. By limiting the quantities of such additives to the minimum desirable while maintaining the functionality of each additive, the impact on the toxicity of the product can be controlled.

In one embodiment, elastomeric matrices of various densities, e.g., from about 0.005 to about 0.15 g/cc (from about 0.31 to about 9.4 lb/ft³) are produced. The density is controlled by, e.g., the amount of blowing or foaming agent, the isocyanate index, the isocyanate component content in the formulation, the reaction exotherm, and/or the pressure of the foaming environment.

Exemplary blowing agents include water and the physical blowing agents, e.g., volatile organic chemicals such as hydrocarbons, ethanol and acetone, and various fluorocarbons and their more environmentally friendly replacements, such as hydrofluorocarbons, chlorofluorocarbons and hydrochlorofluorocarbons. The reaction of water with an isocyanate group yields carbon dioxide, which serves as a blowing agent. Moreover, combinations of blowing agents, such as water with a fluorocarbon, can be used in certain embodiments. In another embodiment, water is used as the blowing agent. Commercial fluorocarbon blowing agents are available from Huntsman, E.I. duPont de Nemours and Co. (Wilmington, Del.), Allied Chemical (Minneapolis, Minn.) and Honeywell (Morristown, N.J.).

For the purpose of this invention, for every 100 parts by weight (or 100 grams) of polyol component (e.g., polycarbonate polyol, polysiloxane polyol) used to make an elastomeric matrix through foaming and crosslinking, the amounts of the other components present, by weight, in a formulation are as follows: from about 10 to about 90 parts (or grams) isocyanate component (e.g., MDIs, their mixtures, H₁₂MDI) with an isocyanate index of from about 0.85 to about 1.10, from about 0.5 to about 5.0 parts (or grams) blowing agent (e.g., water), from about 0.1 to about 0.8 parts (or grams) blowing catalyst (e.g., tertiary amine), from about 0.5 to about 2.5 parts (or grams) surfactant, and from about 0.3 to about 1.0 parts (or grams) cell opener. Of course, the actual amount of isocyanate component used is related to and depends upon the magnitude of the isocyanate index for a particular formulation. Additionally, for every 100 parts by weight (or 100 grams) of polyol component used to make an elastomeric matrix through foaming and crosslinking, the amounts of the following optional components, when present in a formulation, are as follows by weight: up to about 20 parts (or grams) chain extender, up to about 20 parts (or grams) crosslinker, up to about 0.3 parts (or grams) gelling catalyst (e.g., a compound comprising tin), up to about 10.0 parts (or grams) physical blowing agent (e.g., hydrocarbons, ethanol, acetone, fluorocarbons), and up to about 8 parts (or grams) viscosity modifier.

Matrices with appropriate properties for the purposes of the invention, as determined by testing, for example, acceptable compression set at human body temperature, airflow, tensile strength and compressive properties, can then be reticulated.

In another embodiment, the gelling catalyst, e.g., the tin catalyst, is omitted and optionally substituted with another catalyst, e.g., a tertiary amine. In one embodiment, the tertiary amine catalyst comprises one or more non-aromatic amines. In another embodiment, the reaction is conducted so that the tertiary amine catalyst, if employed, is wholly reacted into the polymer, and residues of same are avoided. In another embodiment, the gelling catalyst is omitted and, instead, higher foaming temperatures are used.

In another embodiment, to enhance biodurability and biocompatibility, ingredients for the polymerization process are selected so as to avoid or minimize the presence in the end product elastomeric matrix of biologically adverse substances or substances susceptible to biological attack.

An alternative preparation embodiment pursuant to the invention involves partial or total replacement of water as a blowing agent with water-soluble spheres, fillers or particles which are removed, e.g., by washing, extraction or melting, after full crosslinking of the matrix.

Reticulation of Elastomeric Matrices

Elastomeric matrix 10 can be subjected to any of a variety of post-processing treatments to enhance its utility, some of which are described herein and others of which will be apparent to those skilled in the art. In one embodiment, reticulation of a porous product of the invention, if not already a part of the described production process, may be used to remove at least a portion of any existing interior “windows”, i.e., the residual cell walls 22 illustrated in FIG. 1. Reticulation tends to increase porosity and fluid permeability.

Porous or foam materials with some ruptured cell walls are generally known as “open-cell” materials or foams. In contrast, porous materials from which many, i.e., at least about 50%, of the cell walls have been removed are known as “reticulated” or “at least partially reticulated”. Porous materials from which more, i.e., at least about 65%, of the cell walls have been removed are known as “further reticulated”. If most, i.e., at least about 80%, or substantially all, i.e., at least about 90%, of the cell walls have been removed then the porous material that remains is known as “substantially reticulated” or “fully reticulated”, respectfully. It will be understood that, pursuant to this art usage, a reticulated material or foam comprises a network of at least partially open interconnected cells, thereby excluding the non-reticulated polyether or polycarbonate polyurethanes disclosed by Brady '550.

“Reticulation” generally refers to a process for removing such cell walls not merely rupturing them by a process of crushing. Moreover, undesirable crushing creates debris that must be removed by further processing. Reticulation may be effected, for example, by dissolving out the cell walls, known variously as “chemical reticulation” or “solvent reticulation”; or by burning or exploding out the cell walls, known variously as “combustion reticulation”, “thermal reticulation” or “percussive reticulation”. In one embodiment, such a procedure may be employed in the processes of the invention to reticulate elastomeric matrix 10. In another embodiment, reticulation is accomplished through a plurality of reticulation steps. In another embodiment, two reticulation steps are used. In another embodiment, a first combustion reticulation is followed by a second combustion reticulation. In another embodiment, combustion reticulation is followed by chemical reticulation. In another embodiment, chemical reticulation is followed by combustion reticulation. In another embodiment, a first chemical reticulation is followed by a second chemical reticulation.

In one embodiment relating to vascular malformation applications and the like, the elastomeric matrix can be reticulated to provide an interconnected pore structure, the pores having an average diameter or other largest transverse dimension of at least about 100 μm. In another embodiment, the reticulated elastomeric matrix has pores with average diameter or other largest transverse dimension of at least about 150 μm. In another embodiment, the elastomeric matrix can be reticulated to provide pores with an average diameter or other largest transverse dimension of at least about 250 μm. In another embodiment, the elastomeric matrix can be reticulated to provide pores with an average diameter or other largest transverse dimension of greater than about 250 μm. In another embodiment, the elastomeric matrix can be reticulated to provide pores with an average diameter or other largest transverse dimension of greater than 250 μm. In another embodiment, the elastomeric matrix can be reticulated to provide pores with an average diameter or other largest transverse dimension of at least about 275 μm. In another embodiment, the elastomeric matrix can be reticulated to provide pores with an average diameter or other largest transverse dimension of greater than about 275 μm. In another embodiment, the elastomeric matrix can be reticulated to provide pores with an average diameter or other largest transverse dimension of greater than 275 μm. In another embodiment, the elastomeric matrix can be reticulated to provide pores with an average diameter or other largest transverse dimension of at least about 300 μm. In another embodiment, the elastomeric matrix can be reticulated to provide pores with an average diameter or other largest transverse dimension of greater than about 300 μm. In another embodiment, the elastomeric matrix can be reticulated to provide pores with an average diameter or other largest transverse dimension of greater than 300 μm.

In another embodiment relating to vascular malformation applications and the like, the elastomeric matrix can be reticulated to provide pores with an average diameter or other largest transverse dimension of not greater than about 900 μm. In another embodiment, the elastomeric matrix can be reticulated to provide pores with an average diameter or other largest transverse dimension of not greater than about 850 μm. In another embodiment, the elastomeric matrix can be reticulated to provide pores with an average diameter or other largest transverse dimension of not greater than about 800 μm. In another embodiment, the elastomeric matrix can be reticulated to provide pores with an average diameter or other largest transverse dimension of not greater than about 700 μm. In another embodiment, the elastomeric matrix can be reticulated to provide pores with an average diameter or other largest transverse dimension of not greater than about 600 μm. In another embodiment, the elastomeric matrix can be reticulated to provide pores with an average diameter or other largest transverse dimension of not greater than about 500 μm.

In another embodiment relating to vascular malformation applications and the like, the elastomeric matrix can be reticulated to provide pores with an average diameter or other largest transverse dimension of from about 100 μm to about 900 μm. In another embodiment relating to vascular malformation applications and the like, the elastomeric matrix can be reticulated to provide pores with an average diameter or other largest transverse dimension of from about 100 μm to about 850 μm. In another embodiment relating to vascular malformation applications and the like, the elastomeric matrix can be reticulated to provide pores with an average diameter or other largest transverse dimension of from about 100 μm to about 800 μm. In another embodiment relating to vascular malformation applications and the like, the elastomeric matrix can be reticulated to provide pores with an average diameter or other largest transverse dimension of from about 100 μm to about 700 μm. In another embodiment, the elastomeric matrix can be reticulated to provide pores with an average diameter or other largest transverse dimension of from about 150 μm to about 600 μm. In another embodiment, the elastomeric matrix can be reticulated to provide pores with an average diameter or other largest transverse dimension of from about 200 μm to about 500 μm. In another embodiment, the elastomeric matrix can be reticulated to provide pores with an average diameter or other largest transverse dimension of greater than about 250 μm to about 900 μm. In another embodiment, the elastomeric matrix can be reticulated to provide pores with an average diameter or other largest transverse dimension of greater than about 250 μm to about 850 μm. In another embodiment, the elastomeric matrix can be reticulated to provide pores with an average diameter or other largest transverse dimension of greater than about 250 μm to about 800 μm. In another embodiment, the elastomeric matrix can be reticulated to provide pores with an average diameter or other largest transverse dimension of greater than about 250 μm to about 700 μm. In another embodiment, the elastomeric matrix can be reticulated to provide pores with an average diameter or other largest transverse dimension of greater than about 250 μm to about 600 μm. In another embodiment, the elastomeric matrix can be reticulated to provide pores with an average diameter or other largest transverse dimension of from about 275 μm to about 900 μm. In another embodiment, the elastomeric matrix can be reticulated to provide pores with an average diameter or other largest transverse dimension of from about 275 μm to about 850 μm. In another embodiment, the elastomeric matrix can be reticulated to provide pores with an average diameter or other largest transverse dimension of from about 275 μm to about 800 μm. In another embodiment, the elastomeric matrix can be reticulated to provide pores with an average diameter or other largest transverse dimension of from about 275 μm to about 700 μm. In another embodiment, the elastomeric matrix can be reticulated to provide pores with an average diameter or other largest transverse dimension of from about 275 μm to about 600 μm.

Optionally, the reticulated elastomeric matrix may be purified, for example, by solvent extraction, either before or after reticulation. Any such solvent extraction or other purification process is, in one embodiment, a relatively mild process which is conducted so as to avoid or minimize possible adverse impact on the mechanical or physical properties of the elastomeric matrix that may be necessary to fulfill the objectives of this invention.

One embodiment employs chemical reticulation, where the elastomeric matrix is reticulated in an acid bath comprising an inorganic acid. Another embodiment employs chemical reticulation, where the elastomeric matrix is reticulated in a caustic bath comprising an inorganic base. Another embodiment employs chemical reticulation at an elevated temperature. Another chemical reticulation embodiment employs solvent, sometimes known as solvent reticulation, where a volatile solvent that leaves no residue is used in the process. In another embodiment, a polycarbonate polyurethane is solvent reticulated with a solvent selected from tetrahydrofuran (“THF”), dimethyl acetamide (“DMAC”), dimethyl sulfoxide (“DMSO”), dimethylformamide (“DMF”), N-methyl-2-pyrrolidone, also known as m-pyrol, and their mixtures. In another embodiment, a polycarbonate polyurethane is solvent reticulated with THF. In another embodiment, a polycarbonate polyurethane is solvent reticulated with N-methyl-2-pyrrolidone. In another embodiment, a polycarbonate polyurethane is chemically reticulated with a strong base. In another embodiment, the pH of the strong base is at least about 9.

In any of these chemical reticulation embodiments, the reticulated foam can optionally be washed. In any of these chemical reticulation embodiments, the reticulated foam can optionally be dried.

In one embodiment, combustion reticulation may be employed in which a combustible atmosphere, e.g., a mixture of hydrogen and oxygen, is ignited, e.g., by a spark. In another embodiment, combustion reticulation is conducted in a pressure chamber. In another embodiment, the pressure in the pressure chamber is substantially reduced, e.g., to below about 150-100 millitorr by evacuation for at least about 2 minutes, before hydrogen, oxygen or a mixture thereof is introduced. In another embodiment, the pressure in the pressure chamber is substantially reduced in more than one cycle, e.g., the pressure is substantially reduced, an unreactive gas such as argon or nitrogen is introduced then the pressure is again substantially reduced, before hydrogen, oxygen or a mixture thereof is introduced. The temperature at which reticulation occurs can be influenced by, e.g., the temperature at which the chamber is maintained and/or by the hydrogen/oxygen ratio in the chamber. In another embodiment, combustion reticulation is followed by an annealing period. In any of these combustion reticulation embodiments, the reticulated foam can optionally be washed. In any of these combustion reticulation embodiments, the reticulated foam can optionally be dried.

In one embodiment, the reticulation process is conducted to provide an elastomeric matrix configuration favoring cellular ingrowth and proliferation into the interior of the matrix. In another embodiment, the reticulation process is conducted to provide an elastomeric matrix configuration which favors cellular ingrowth and proliferation throughout the elastomeric matrix configured for implantation, as described herein.

The term “configure” and its derivative terms are used to denote the arranging, shaping and dimensioning of the respective structure to which the term is applied. Thus, reference to a structure as being “configured” for a purpose is intended to reference the whole spatial geometry of the relevant structure or part of a structure as being selected or designed to serve the stated purpose.

Reticulated Elastomeric Matrices by Sacrificial Molding

In general, suitable elastomer materials for use in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Of particular interest are thermoplastic elastomers such as polyurethanes whose chemistry is associated with good biodurability properties, for example. In one embodiment, such thermoplastic polyurethane elastomers include polycarbonate polyurethanes, polyester polyurethanes, polyether polyurethanes, polysiloxane polyurethanes, hydrocarbon polyurethanes (i.e., those thermoplastic elastomer polyurethanes formed from at least one isocyanate component comprising, on the average, about 2 isocyanate groups per molecule and at least one hydroxy-terminated hydrocarbon oligomer and/or hydrocarbon polymer), polyurethanes with so-called “mixed” soft segments, and mixtures thereof. Mixed soft segment polyurethanes are known to those skilled in the art and include, e.g., polycarbonate-polyester polyurethanes, polycarbonate-polyether polyurethanes, polycarbonate-polysiloxane polyurethanes, polycarbonate-hydrocarbon polyurethanes, polycarbonate-polysiloxane-hydrocarbon polyurethanes, polyester-polyether polyurethanes, polyester-polysiloxane polyurethanes, polyester-hydrocarbon polyurethanes, polyether-polysiloxane polyurethanes, polyether-hydrocarbon polyurethanes, polyether-polysiloxane-hydrocarbon polyurethanes and polysiloxane-hydrocarbon polyurethanes. In another embodiment, the thermoplastic polyurethane elastomer includes polycarbonate polyurethanes, polyether polyurethanes, polysiloxane polyurethanes, hydrocarbon polyurethanes, polyurethanes with these mixed soft segments, or mixtures thereof. In another embodiment, the thermoplastic polyurethane elastomer includes polycarbonate polyurethanes, polysiloxane polyurethanes, hydrocarbon polyurethanes, polyurethanes with these mixed soft segments, or mixtures thereof. In another embodiment, the thermoplastic polyurethane elastomer is a polycarbonate polyurethane, or mixtures thereof. In another embodiment, the thermoplastic polyurethane elastomer is a polysiloxane polyurethane, or mixtures thereof. In another embodiment, the thermoplastic polyurethane elastomer is a polysiloxane polyurethane, or mixtures thereof. In another embodiment, the thermoplastic polyurethane elastomer comprises at least one diisocyanate in the isocyanate component, at least one chain extender and at least one diol, and may be formed from any combination of the diisocyanates, difunctional chain extenders and diols described in detail above.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some suitable thermoplastics for practicing the invention, in one embodiment suitably characterized as described herein, can include: polyolefinic polymers with alternating secondary and quaternary carbons as disclosed by Pinchuk et al. in U.S. Pat. No. 5,741,331 (and its divisional U.S. Pat. Nos. 6,102,939 and 6,197,240); block copolymers having an elastomeric block, e.g., a polyolefin, and a thermoplastic block, e.g., a styrene, as disclosed by Pinchuk et al. in U.S. patent application Publication No. 2002/0107330 A1; thermoplastic segmented polyetherester, thermoplastic polydimethylsiloxane, di-block polystyrene polybutadiene, tri-block polystyrene polybutadiene, poly(acrylene ether sulfone)-poly(acryl carbonate) block copolymers, di-block copolymers of polybutadiene and polyisoprene, copolymers of ethylene vinyl acetate (EVA), segmented block co-polystyrene polyethylene oxide, di-block co-polystyrene polyethylene oxide, and tri-block co-polystyrene polyethylene oxide, e.g., as disclosed by Penhasi in U.S. patent application Publication No. 2003/0208259 A1 (particularly, see paragraph [0035] therein); and polyurethanes with mixed soft segments comprising polysiloxane together with a polyether and/or a polycarbonate component, as disclosed by Meijs et al. in U.S. Pat. No. 6,313,254; and those polyurethanes disclosed by DiDomenico et al. in U.S. Pat. Nos. 6,149,678, 6,111,052 and 5,986,034. However, a careful reading of Brady '550 indicates that the polyether or polycarbonate polyurethanes having isocyanurate linkages disclosed therein are not suitable because, inter alia, they are not thermoplastic. Also suitable for use in practicing the present invention are novel or known elastomers synthesized by a process according to the invention, as described herein. In another embodiment, an optional therapeutic agent may be loaded into the appropriate block of other elastomers used in the practice of the invention.

Some commercially-available thermoplastic elastomers suitable for use in practicing the present invention include the line of polycarbonate polyurethanes supplied under the trademark BIONATE® by The Polymer Technology Group Inc. (Berkeley, Calif.). For example, the very well-characterized grades of polycarbonate polyurethane polymer BIONATE® 80A, 55 and 90 are soluble in THF, processable, reportedly have good mechanical properties, lack cytotoxicity, lack mutagenicity, lack carcinogenicity and are non-hemolytic. Another commercially-available elastomer suitable for use in practicing the present invention is the CHRONOFLEX® C line of biodurable medical grade polycarbonate aromatic polyurethane thermoplastic elastomers available from CardioTech International, Inc. (Woburn, Mass.). Yet another commercially-available elastomer suitable for use in practicing the present invention is the PELLETHANE® line of thermoplastic polyurethane elastomers, in particular the 2363 series products and more particularly those products designated 81A and 85A, supplied by The Dow Chemical Company (Midland, Mich.). These commercial polyurethane polymers are linear, not crosslinked, polymers, therefore, they are soluble, readily analyzable and readily characterizable.

Sacrificial Molding Process

The following sacrificial molding process may be performed using any of the thermoplastic elastomers described above as the flowable polymeric material or as a component thereof. In one embodiment, the flowable polymeric material in the sacrificial molding process comprises a polycarbonate polyurethane.

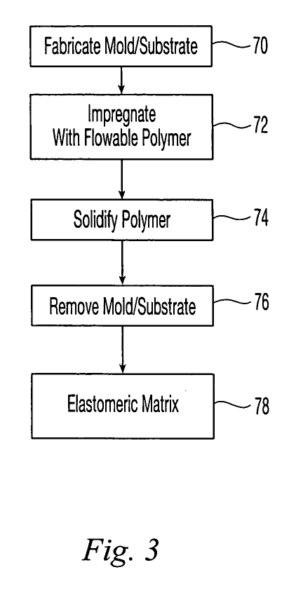

Referring now to the sacrificial molding process for preparing a reticulated biodurable elastomeric matrix illustrated in FIG. 3, the process comprises an initial step 70 of fabricating a sacrificial mold or substrate permeated with externally communicating interconnecting interior passageways, which interior passageways are shaped, configured and dimensioned to define or mold the elastomeric matrix with a desired reticulated microstructural configuration.

The substrate or sacrificial mold can comprise a plurality of solid or hollow beads or particles agglomerated, or interconnected one with another at multiple points on each particle in the manner of a network. In another embodiment, the mold may comprise a plurality of waxy particles compressed together so that each particle contacts its neighbors at multiple points, for example, 4 to 8 points for interior particles, i.e., those in the interior and not at the surface of the mold. In another embodiment, the particles are symmetrical, but they may have any suitable shape, e.g., an isotropically symmetrical shape, for example, dodecahedral, icosahedral or spherical. In one embodiment, before compaction, the particles are spherical, each with a diameter of from about 0.5 mm to about 6 mm. In another embodiment, the mold may comprise a plurality of particles comprising a material having water solubility, for example, an inorganic salt such as sodium chloride or calcium chloride, or a starch such as corn, potato, wheat, tapioca, manioc or rice starch.

The starch can be obtained from, e.g., corn or maize, potatoes, wheat, tapioca, manioc and/or rice, by methods known to those in the art. In one embodiment the starch is a mixture of starches. In another embodiment the starch contains from about 99 wt. % to about 70 wt. % amylopectin. In another embodiment the starch contains about 80 wt. % amylopectin and about 20 wt. % amylose. Suitable granular starches include the modified rice starches REMYLINE DR (available from ABR Lundberg, Malmo, Sweden) and MIKROLYS 54 (available from Lyckeby Starkelse AB, Sweden), the PHARMGEL line of starches and modified starches available from the Cerestar Food & Pharma division of Cargill (Cedar Rapids, Iowa), the wheat starch ABRASTARCH (ABR Foods Ltd., Northamptonshire, UK), and the corn starches HYLON VII, HYLON V, and AMIOCA (each from National Starch and Chemical Co., Bridgewater, N.J.). The desired particle size of the starch can be achieved by methods known to those in the art. For example, the starch particles can be sieved to the desired size, water can be used to agglomerate small starch particles into larger particles, or a binder can be used to agglomerate small starch particles into larger particles, e.g., as disclosed in U.S. Pat. No. 5,726,161. In another embodiment, an aqueous solution or suspension of starch particles can be placed into the pores of a reticulated foam structure (a “positive”), e.g., a non-medical grade commercial foam formed from polyurethane, the starch can be gelatinized as described below, the sample can be dried under reduced pressure and/or baked to remove water, and the foam removed by dissolving it with a solvent, e.g., THF for a polyurethane foam, that is also a nonsolvent for the starch, thereby yielding a starch assembly (a “negative”) that can be readily fabricated into starch particles having an average diameter about that of the pore diameter of the starting reticulated foam structure.

Optionally, the particles may be interconnected using heat and/or pressure, e.g., by sintering or fusing. However, if there is some conformation at the contact points under pressure, the application of heat may not be necessary. In one embodiment, the particles are interconnected by sintering, by fusing, by using an adhesive, by the application of reduced pressure, or by any combination thereof. In one embodiment, waxy particles are fused together by raising their temperature. In another embodiment, starch particles are fused together by raising their temperature. In another embodiment, inorganic salt particles are fused together by exposing them to moisture, e.g., 90% relative humidity. In another embodiment, starch particles are fused or gelatinized by heating, in one embodiment from about 2 hours to about 4 hours, in one embodiment to from about 50° C. to about 100° C., in another embodiment to from about 70° C. to about 90° C., an aqueous starch solution or suspension, e.g., as disclosed in column 4, lines 1-7 of U.S. Pat. No. 6,169,048 B1. In another embodiment, resilient particles may be employed provided that they can be eluted from the matrix, for example, by elevating their temperature to liquefy them, by dissolving them with a solvent or solvent blend, or by elevating their temperature and dissolving them. In one embodiment, the mold has a significant three-dimensional extent with multiple particles extending in each dimension. In another embodiment, the polymeric material is contained within the interstices between the interconnected particles. In another embodiment, the polymeric material fills the interstices between the interconnected particles.

In one embodiment, the particles comprise a material having a melting point at least 5° C. lower than the softening temperature of the polymer that is contained within the interstices. In another embodiment, the particles comprise a material having a melting point at least 10° C. lower than the softening temperature of the polymer that is contained within the interstices. In another embodiment, the particles comprise a material having a melting point at least 20° C. lower than the softening temperature of the polymer that is contained within the interstices. In another embodiment, the particles comprise a material having a melting point at least 5° C. lower than the Vicat softening temperature of the polymer that is contained within the interstices. In another embodiment, the particles comprise a material having a melting point at least 10° C. lower than the Vicat softening temperature of the polymer that is contained within the interstices. In another embodiment, the particles comprise a material having a melting point at least 20° C. lower than the Vicat softening temperature of the polymer that is contained within the interstices. For example, the particles of the mold may be a hydrocarbon wax. In another embodiment, the removed particle material can be recovered after melting and reformed into particles for reuse.

In another embodiment, the particles comprise an inorganic salt which may be removed by dissolving the salt in water. In another embodiment, the particles comprise a starch which may be removed by dissolving the starch in a solvent for the starch. In another embodiment, the particles comprise a starch which may be removed by dissolving the starch in water. In another embodiment, the particles comprise a starch which may be removed by dissolving the starch in an aqueous base, such as aqueous NaOH. In another embodiment, the particles comprise a starch which may be removed by dissolving the starch in about 1-5 M aqueous NaOH, in another embodiment about 2.5-3 M NaOH, in another embodiment about 2.5 M NaOH. In another embodiment, the aqueous base further comprises sodium sulfate. In another embodiment, the particles comprise a starch which may be removed by the enzymatic action of an enzyme, as known to those in the art. For example, the enzyme can be an alpha-amylase (E.C. 3.2.1.1), pullulanase (E.C. 3.2.1.41), isoamylase (E.C. 3.2.1.68), amyloglucosidase (E.C. 3.2.1.3), sometimes known as glucoamylase, and the like, and mixtures thereof. Such enzymes are disclosed in, e.g., U.S. Pat. No. 6,569,653 B1 and column 1, line 50 to column 2, line 14 of U.S. Pat. No. 6,448,049 B1. Suitable alpha-amylases include the TERMAMYL 120L S, L and LS types (Novo Nordisk Bioindustries S.A., Nanterre, France), SPEZYME AA and AAL (Genencor, Delft, Netherlands), and NERVANASE and G-ZYME G995 (Rhodia, Cheshire, UK); suitable pullulanases include AMBAZYME P20 (Rhodia), PROMOZYME 200 L (Novo Nordisk), and OPTIMAX L300 (Genencor); and suitable amyloglucosidases include OPTIDEX L300 and OPTIMAX 7525 (Genencor), AMG 300L (Novo Nordisk), and other enzymes cited at column 5, lines 7-19 of U.S. Pat. No. 6,569,653 B1.

In embodiments where the substrate is hydrophobic, it may be given an amphiphilic coating to induce hydrophilicity in the surface of the elastomer as it sets. For example hydrocarbon wax particles, may be coated with a detergent, lecithin, functionalized silicones, or the like.

In one embodiment, the substrate comprises two phases: a substrate material phase and a spatial phase. The substrate material phase comprises a three-dimensionally extending network of substrate particles, continuously interconnecting one with the next, interspersed with a three-dimensionally extending network of interstitial spaces also continuously interconnecting one with another and which will be filled with polymeric material to provide a single structural matrix constituting the porous elastomeric matrix.

The substrate defines the spaces that will constitute pores in the end product reticulated elastomeric matrix.

In the next step, step 72, the process comprises charging the mold or impregnating the substrate with a flowable polymeric material. The flowable polymeric material may be a polymer solution, emulsion, microemulsion, suspension, dispersion, a liquid polymer, or a polymer melt. For example, the flowable polymeric material can comprise a solution of the polymer in a volatile organic solvent, for example THF.

In one embodiment, the polymeric material can comprise a thermoplastic elastomer and the flowable polymeric material can comprise a solution of that thermoplastic elastomer. In another embodiment, the polymeric material can comprise a biodurable thermoplastic elastomer, as described herein, and the flowable polymeric material can comprise a solution of that biodurable thermoplastic elastomer. In another embodiment, the polymeric material can comprise a solvent-soluble biodurable thermoplastic elastomer and the flowable polymeric material can comprise a solution of that solvent-soluble biodurable thermoplastic elastomer. The solvent can then be removed or allowed to evaporate to solidify the polymeric material. Suitable elastomers include the BIONATE® line of polyurethane elastomers. Others are described herein or will be known or apparent to those skilled in the art.

In one embodiment, solvents are biocompatible and sufficiently volatile to be readily removed. One suitable solvent, depending, of course, upon the solubility of the polymer, is THF. Other suitable solvents include DMAC, DMF, DMSO and N-methyl-2-pyrrolidone. Additionally, solvent mixtures can be used, e.g., mixtures of at least two of THF, DMAC, DMF, DMSO and N-methyl-2-pyrrolidone. Additional suitable solvents will be known to those skilled in the art.

The sacrificial molding process further comprises solidifying the polymeric material, step 74, which may be effected in any desired manner, for example, by solvent exchange or by removing the solvent by evaporation, optionally assisted by vacuum and/or heating to a temperature below the softening temperatures of the polymer or of the substrate material. If sufficiently volatile, the solvent may be allowed to evaporate off, e.g., overnight. The product resulting from step 74 is a solid complex comprising interspersed polymer material and substrate.

Removing the substrate, step 76, for example, by melting, dissolving, subliming or enzymatically removing it, yields the reticulated elastomeric matrix 78. In one embodiment, the matrix comprises interconnecting cells each defined by one of the removed particles. Most or many of the cells are open-walled to provide matrix 78 with good fluid permeability. In another embodiment, matrix 78 may be reticulated to provide a reticulated matrix. In another embodiment, for endovascular applications, the matrix is fully reticulated with few, if any residual cell walls.

In many embodiments of the sacrificial molding process discussed above, the structure of elastomeric matrix 10 that is produced without the need to employ a separate reticulation process step is, in one embodiment, a “reticulated” or an “at least partially reticulated” one, i.e., at least about 50% of the cell walls are absent. In other embodiments, the structure of elastomeric matrix 10 that is produced without the need to employ a separate reticulation process step is a “further reticulated” one, i.e., at least about 65% of the cell walls are absent. In other embodiments, the structure of elastomeric matrix 10 that is produced without the need to employ a separate reticulation process step is a “substantially reticulated” one, i.e., at least about 80% of the cell walls are absent. In other embodiments, the structure of elastomeric matrix 10 that is produced without the need to employ a separate reticulation process step is a “fully reticulated” one, i.e., at least about 90% of the cell walls are absent. However, in another embodiment, an optional reticulation step may be performed on the matrix prepared by any of the processes described herein, to open smaller pores and eliminate at least some residual cell walls. For example, if, in a particular embodiment, the viscosity of the polymer solution limits the extent to which the polymer solution can permeate some of the smaller channels between particles 80, sintering or fusing of the particles may be limited and the “windows” or cell walls that result optionally can be blown out by reticulation, as discussed below.

Optionally, the elastomeric matrix 10 resulting from the sacrificial molding process can be annealed for structural stabilization and/or to increase its degree of crystallinity and/or to increase its crystalline melting point. Exemplary annealing conditions include heating the elastomeric matrix to a temperature of from about 35° C. to about 150° C. and maintaining the elastomeric matrix in that temperature range for about 2 hours to about 24 hours.

The sacrificial molding process is further described in Examples 1 through 5.

Double Lost Wax Process

The invention also provides what may, for simplicity's sake and without limitation, be thought of as a so-called “double lost wax process” for producing a reticulated biodurable elastomeric matrix 10. As a brief, non-limiting summary of this process, a template of the desired product shape is obtained and coated with a first coating. The template is removed and the coating is then coated with a second coating of the final polymer material. When the first coating is removed, the desired product made from the final polymer material remains. Since two materials, the template and the first coating, are each removed in a separate process step, such process is known as a so-called “double lost wax process” even though neither the template nor the first coating need necessarily comprise a wax. For example, the first coating can be formed from a starch, such as those previously described, by depositing an aqueous starch solution or suspension onto or into the template then performing a starch gelatinization step, as previously described, optionally followed by removal of the water.

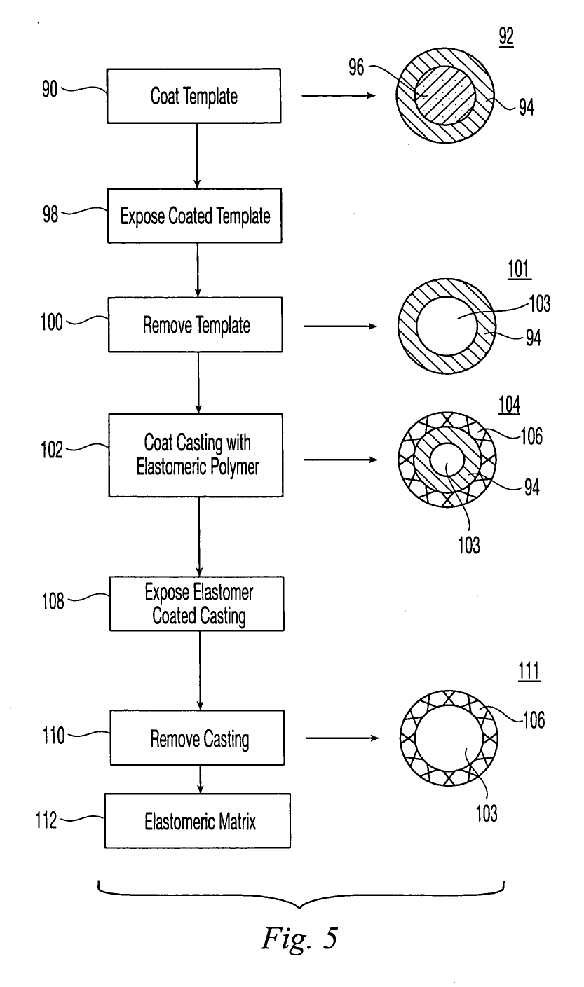

A desirable template would be a commercial reticulated crosslinked foam, e.g., a non-biodurable polyurethane. However, this may be impractical because if such crosslinked foam is directly coated, e.g., with a flowable thermoplastic elastomer such as one from the BIONATE® or CHRONOFLEX® product lines described above, the crosslinked reticulated template, being crosslinked, cannot be easily removed: If a strong acidic or caustic extraction of the crosslinked foam template were to be attempted, thereby destructively converting it into a solution, such extraction could also dissolve or destroy the thermoplastic elastomer coating. One embodiment of the present invention solves this problem by using an intermediate lost wax coating. In this so-called double lost wax process embodiment, a foam template, e.g., a reticulated polyurethane foam that may be non-biodurable, is first coated with a flowable resistant material, e.g., a solution comprising a material resistant to attack by a strong hot acid or base to be employed for dissolution of the foam template or a liquid form of the resistant material. For example, the resistant material of the first coating can comprise a solvent-soluble but acid- or base-insoluble thermoplastic polymer or wax. Then, the foam template is removed, e.g., by extraction with hot acid or base, leaving a shell-like resistant material form which is then coated with a flowable polymeric material such as flowable form of the desired solid phase 12, e.g., a solution of biodurable polyurethane in a solvent, as the second coating. Removal of the resistant first coating material, e.g., by solvent-extracting, melting-out or subliming-away the wax, yields a reticulated biodurable polyurethane elastomeric matrix. An example of this process is illustrated schematically in FIG. 5.

The following double lost wax process may be performed using any of the thermoplastic elastomers described above as the flowable elastomeric polymeric material or as a component thereof. In one embodiment, the flowable elastomeric polymeric material in the double lost wax process comprises a polycarbonate polyurethane.

Referring to FIG. 5, the illustrated double lost wax process comprises an initial step 90 of coating a reticulated foam template formed, for example, of the polyurethane CREST FOAM™ grade S-20 (available from Crest Foam, Inc., Moonachie, N.J.), with a solvent-soluble, readily meltable or sublimable thermoplastic or wax, such as polystyrene, polyvinyl chloride, paraffin wax or the like, applied from the melt or solution of the thermoplastic or wax. As shown in FIG. 5, a cross-sectional view of, e.g., a cylindrical strut section 92 of the coated foam product of step 90, comprises a ring 94 of wax around a core 96 of the foam template.

In the next step, step 98, any solvent is removed, e.g., by drying, and a surface of the polyurethane core material of the coated reticulated foam template is exposed, e.g., by cutting.

In step 100, the polyurethane foam template is removed, e.g., by dissolving it using hot acid or base, to yield a wax casting of the reticulated foam core. As shown in FIG. 5, a cross-sectional view of, e.g., a cylindrical strut section 102 of the casting, comprises a hollow ring 94 of wax.

The next process step, step 102, comprises coating the wax casting with a flowable elastomeric polymeric material, such as a solution or melt of a biodurable polyurethane elastomer, e.g., one of the grades supplied under the trademarks CHRONOFLEX® and BIONATE®. A cross-sectional view of, e.g., a cylindrical strut section 104 of the elastomer-coated wax casting product of step 102, comprises a biodurable elastomer ring 106 around a core comprising wax ring 94. The flowable elastomeric polymeric material is then solidified by, e.g., removing the solvent of a solution or cooling a polymer melt.

The next step, step 108, comprises exposing the thermoplastic or wax, e.g., by cutting the elastomeric polymer matrix.

In step 110, the thermoplastic or wax is removed, e.g., by melting, dissolving or subliming-away the casting, to yield an elastomeric polymer material matrix shown a cross-sectional view of, e.g., a cylindrical strut section, as ring 112.

Reticulated Elastomeric Matrices by Lyophilization

In one embodiment, a biodurable reticulated elastomeric matrix of the invention can be made by lyophilizing a flowable polymeric material. In another embodiment, the polymeric material comprises a solution of a solvent-soluble biodurable elastomer in a solvent. The flowable polymeric material is subjected to a lyophilization process comprising solidifying the flowable polymeric material to form a solid, e.g., by cooling a solution, then removing the non-polymeric material, e.g., by subliming the solvent from the solid under reduced pressure, to provide an at least partially reticulated elastomeric matrix. The density of the at least partially reticulated elastomeric matrix is less than the density of the starting polymeric material. In another embodiment, a solution of a biodurable elastomer in a solvent is substantially, but not necessarily completely, solidified, then the solvent is sublimed from that material to provide an at least partially reticulated elastomeric matrix. By selecting the appropriate solvent or solvent mixture to dissolve the polymer, aided by agitation and/or the application of heat, a homogeneous solution amenable to lyophilization can be obtained by a suitable mixing process. In another embodiment, the temperature to which the solution is cooled is below the freezing temperature of the solution. In another embodiment, the temperature to which the solution is cooled is above the apparent glass transition temperature of the solid and below the freezing temperature of the solution.

Without being bound by any particular theory, it is thought that, during lyophilization, a polymer solution separates in a controlled manner into either two distinct phases, e.g., one phase, i.e., the solvent, being continuous and the other phase being dispersed in the continuous phase, or into two bicontinuous phases. In each case, subsequent removal of the solvent phase results in a porous structure with a range or distribution of pore sizes. These pores are usually interconnected. Their shape, size and orientation depend upon the properties of the solution and the lyophilization processing conditions in conventional ways. For example, a lyophilization product has a range of pore sizes with dimensions that can be changed by altering, e.g., the freezing temperature, freezing rate, nucleation density, polymer concentration, polymer molecular weight, and the type of solvent(s) in ways known to those in the art.

Some commercially-available thermoplastic elastomers suitable for use in practicing lyophilization for the present invention include but are not limited to those discussed above in connection with obtaining reticulated elastomeric matrices by the sacrificial molding process. Moreover, in another embodiment, polyurethane thermoplastic elastomers having mixed soft segments comprising polysiloxane together with a polyether and/or a polycarbonate component, as disclosed by Meijs et al. in U.S. Pat. No. 6,313,254, can be used.

Solvents for use in practicing lyophilization for the present invention include but are not limited to THF, DMAC, DMSO, DMF, cyclohexane, ethanol, dioxane, N-methyl-2-pyrrolidone, and their mixtures. Generally, the amount of polymer in the solution is from about 0.5% to about 30% of the solution by weight in one embodiment, depending upon the solubility of the polymer in the solvent and the final desired properties of the elastomeric reticulated matrix. In another embodiment, the amount of polymer in the solution is from about 0.5% to about 15% of the solution by weight.

Additionally, additives may be present in the polymer-solvent solution, e.g., a buffer. In one embodiment, the additive does not react with the polymer or the solvent. In another embodiment, the additive is a solid material that promotes tissue regeneration or regrowth, a buffer, a reinforcing material, a porosity modifier or a pharmaceutically-active agent.

In another embodiment, the polymer solution can comprise various inserts incorporated with the solution, such as films, plates, foams, scrims, woven, nonwoven, knitted or braided textile structures, or implants that have surfaces that are not smooth. In another embodiment, the solution can be prepared in association with a structural insert such as an orthopedic, urological or vascular implant. In another embodiment, these inserts comprise at least one biocompatible material and may have a non-absorbability and/or absorbability aspect.

The type of pore morphology that becomes locked-in during the freezing step and that is present in the reticulated elastomeric matrix remaining after the solvent is removed is a function of, e.g., the solution thermodynamics, freezing rate and temperature to which the solution is cooled, polymer concentration in the solution and type of nucleation, e.g., homogeneous or heterogeneous. In one embodiment, the lyophilizer for the polymer solution is cooled to about −80° C. In another embodiment, the lyophilizer for the polymer solution is cooled to about −70° C. In another embodiment, the lyophilizer for the polymer solution is cooled to about −40° C. In one embodiment, the lyophilizer comprises a shelf onto which the polymer solution is placed and the shelf is cooled to about −80° C. In another embodiment, the shelf is cooled to about −70° C. In another embodiment, the shelf is cooled to about −40° C. The rate of cooling to freeze the polymer solution can be from about 0.2° C./min to about 2.5° C./min.

At the start of the lyophilization process, the polymer solution is placed into a mold and the mold is placed into the lyophilizer. The walls of the mold undergo cooling in the lyophilizer, e.g., as they contact the freeze-dryer shelf. The temperature of the lyophilizer is reduced at the desired cooling rate until the final cooling temperature is attained. For example, in a lyophilizer where the mold is placed onto a cooled shelf, the heat transfer front moves upwards from the lyophilizer shelf through the mold wall into the polymer solution. The rate at which this front advances influences the nucleation and the orientation of the frozen structure. This rate depends on, e.g., the cooling rate and the thermal conductivity of the mold. When the temperature of the solution goes below the gellation and/or freezing point of the solvent, the solution can phase separate into two distinct phases or into two bicontinuous phases, as discussed previously. The morphology of the phase separated system is locked into place during the freezing step of the lyophilization process. The creation of pores is initiated by the sublimation of the solvent upon exposing the frozen material to reduced pressure.

Without being bound by any particular theory, in general, a higher concentration of the polymer in the solution, higher viscosity (attributable to higher concentration or higher molecular weight of the polymer) or higher cooling rate are thought to lead to smaller pore sizes while lower concentration of the polymer in the solution, lower viscosity (attributable to lower concentration or lower molecular weight of the polymer) or slower cooling rate are thought to lead to larger pore sizes in the lyophilized products.

The lyophilization process is further described in Example 18.

Imparting Endopore Features

Within pores 20, elastomeric matrix 10 may, optionally, have features in addition to the void or gas-filled volume described above. In one embodiment, elastomeric matrix 10 may have what are referred to herein as “endopore” features, i.e., features of elastomeric matrix 10 that are located “within the pores”. In one embodiment, the internal surfaces of pores 20 may be “endoporously coated”, i.e., coated or treated to impart to those surfaces a degree of a desired characteristic, e.g., hydrophilicity. The coating or treating medium can have additional capacity to transport or bond to active ingredients that can then be preferentially delivered to pores 20. In one embodiment, this coating medium or treatment can be used facilitate covalent bonding of materials to the interior pore surfaces, for example, as are described in the copending applications. In another embodiment, the coating comprises a biodegradable polymer and an inorganic component, such as hydroxyapatite. Hydrophilic treatments may be effected by chemical or radiation treatments on the fabricated reticulated elastomeric matrix 10, by exposing the elastomer to a hydrophilic, e.g., aqueous, environment during elastomer setting, or by other means known to those skilled in the art.

Furthermore, one or more coatings may be applied endoporously by contacting with a film-forming biocompatible polymer either in a liquid coating solution or in a melt state under conditions suitable to allow the formation of a biocompatible polymer film. In one embodiment, the polymers used for such coatings are film-forming biocompatible polymers with sufficiently high molecular weight so as to not be waxy or tacky. The polymers should also adhere to the solid phase 12. In another embodiment, the bonding strength is such that the polymer film does not crack or dislodge during handling or deployment of reticulated elastomeric matrix 10.

Suitable biocompatible polymers include polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone and blends thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules and blends thereof. For the purpose of this invention aliphatic polyesters include polymers and copolymers of lactide (which includes lactic acid d-, 1- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and blends thereof.

Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from crosslinked polyvinyl pyrrolidinone and polyesters. Other polymers, of course, can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydoxyalkyl celluloses); and mixtures thereof. For the purpose of this invention, polyamides include polyamides of the general forms:
—N(H)—(CH₂)ₙ—C(O)— and —N(H)—(CH₂)ₓ—N(H)—C(O)—(CH₂)_{y}—C(O)—,
where n is an integer from about 4 to about 13; x is an integer from about 4 to about 12; and y is an integer from about 4 to about 16. It is, of course, to be understood that the listings of materials above are illustrative but not limiting.

The devices made from reticulated elastomeric matrix 10 generally are coated by simple dip or spray coating with a polymer, optionally comprising a pharmaceutically-active agent, such as a therapeutic agent or drug. In one embodiment, the coating is a solution and the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight.

The solvent or solvent blend for the coating solution is chosen with consideration given to, inter alia, the proper balancing the viscosity, deposition level of the polymer, wetting rate and evaporation rate of the solvent to properly coat solid phase 12, as known to those in the art. In one embodiment, the solvent is chosen such the polymer is soluble in the solvent. In another embodiment, the solvent is substantially completely removed from the coating. In another embodiment, the solvent is non-toxic, non-carcinogenic and environmentally benign. Mixed solvent systems can be advantageous for controlling the viscosity and evaporation rates. In all cases, the solvent should not react with the coating polymer. Solvents include by are not limited to: acetone, N-methylpyrrolidone (“NMP”), DMSO, toluene, methylene chloride, chloroform, 1,1,2-trichloroethane (“TCE”), various freons, dioxane, ethyl acetate, THF, DMF, DMAC, and their mixtures.

In another embodiment, the film-forming coating polymer is a thermoplastic polymer that is melted, enters the pores 20 of the elastomeric matrix 10 and, upon cooling or solidifying, forms a coating on at least a portion of the solid material 12 of the elastomeric matrix 10. In another embodiment, the processing temperature of the thermoplastic coating polymer in its melted form is above about 60° C. In another embodiment, the processing temperature of the thermoplastic coating polymer in its melted form is above about 90° C. In another embodiment, the processing temperature of the thermoplastic coating polymer in its melted form is above about 120° C.

In a further embodiment of the invention, described in more detail below, some or all of the pores 20 of elastomeric matrix 10 are coated or filled with a cellular ingrowth promoter. In another embodiment, the promoter can be foamed. In another embodiment, the promoter can be present as a film. The promoter can be a biodegradable material to promote cellular invasion of elastomeric matrix 10 in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of elastomeric matrix 10 is coated or impregnated, as described in the previous section but substituting the promoter for the biocompatible polymer or adding the promoter to the biocompatible polymer, to encourage cellular ingrowth and proliferation.

In one embodiment, the coating or impregnating process is conducted so as to ensure that the product “composite elastomeric implantable device”, i.e., a reticulated elastomeric matrix and a coating, as used herein, retains sufficient resiliency after compression such that it can be delivery-device delivered, e.g., catheter, syringe or endoscope delivered. Some embodiments of such a composite elastomeric implantable device will now be described with reference to collagen, by way of non-limiting example, with the understanding that other materials may be employed in place of collagen, as described above.

One embodiment of the invention is a process for preparing a composite elastomeric implantable device comprising:
a) infiltrating an aqueous collagen slurry into the pores of a reticulated, porous elastomer, such as elastomeric matrix 10, which is optionally a biodurable elastomer product; and
b) removing the water, optionally by lyophilizing, to provide a collagen coating, where the collagen coating optionally comprises an interconnected network of pores, on at least a portion of a pore surface of the reticulated, porous elastomer.

Collagen may be infiltrated by forcing, e.g., with pressure, an aqueous collagen slurry, suspension or solution into the pores of an elastomeric matrix. The collagen may be Type I, II or III or mixtures thereof. In one embodiment, the collagen type comprises at least 90% collagen I. The concentration of collagen is from about 0.3% to about 2.0% by weight and the pH of the slurry, suspension or solution is adjusted to be from about 2.6 to about 5.0 at the time of lyophilization. Alternatively, collagen may be infiltrated by dipping an elastomeric matrix into a collagen slurry.

As compared with the uncoated reticulated elastomer, the composite elastomeric implantable device can have a void phase 14 that is slightly reduced in volume. In one embodiment, the composite elastomeric implantable device retains good fluid permeability and sufficient porosity for ingrowth and proliferation of fibroblasts or other cells.

Optionally, the lyophilized collagen can be crosslinked to control the rate of in vivo enzymatic degradation of the collagen coating and to control the ability of the collagen coating to bond to elastomeric matrix 10. Without being bound by any particular theory, it is thought that when the composite elastomeric implantable device is implanted, tissue-forming agents that have a high affinity to collagen, such as fibroblasts, will more readily invade the collagen-impregnated elastomeric matrix 10 than the uncoated matrix. It is further thought, again without being bound by any particular theory, that as the collagen enzymatically degrades, new tissue invades and fills voids left by the degrading collagen while also infiltrating and filling other available spaces in the elastomeric matrix 10. Such a collagen coated or impregnated elastomeric matrix 10 is thought, without being bound by any particular theory, to be additionally advantageous for the structural integrity provided by the reinforcing effect of the collagen within the pores 20 of the elastomeric matrix 10, which can impart greater rigidity and structural stability to various configurations of elastomeric matrix 10.

Processes of preparing a collagen-coated composite elastomeric implantable device and a sleeve formed therefrom are described below by way of example in Examples 10 and 11. Other processes will be apparent to those skilled in the art.

Coated Implantable Devices

In some applications, a device made from elastomeric matrix 10 can have a coated or fused surface in order to present a smaller outermost surface area, because the internal surface area of pores below the surface is no longer accessible. Without being bound by any particular theory, it is thought that this decreased surface area provides more predictable and easier delivery and transport through long tortuous channels inside delivery-devices and transport through long tortuous channels inside delivery-devices introduced by percutaneous, minimally-invasive procedures for treatment of vascular malformations, such as aneurysms, arterio venous malfunctions, arterial embolizations or other vascular abnormalities. Further, this increased surface area and the hardness of elastomeric matrix 10 is thought, without being bound by any particular theory, to provoke faster inflammatory response, activate the onset of a coagulation cascade, provoke intimal proliferation, stimulate endothelial cell migration and early onset of restenosis. Surface coating or fusion alters the “porosity of the surface”, i.e., at least partially reduces the percentage of pores open to the surface, or, in the limit, completely closes-off the pores of a coated or fused surface, i.e., that surface is nonporous because it has substantially no pores remaining on the coated or fused surface. However, surface coating or fusion still allows the internal interconnected porous structure of elastomeric matrix 10 to remain open internally and on other non-coated or non-fused surfaces; e.g., the portion of a coated or fused pore not at the surface remains interconnected to other pores, and those remaining open surfaces can foster cellular ingrowth and proliferation. In one embodiment, a coated and uncoated surface are orthogonal to each other. In another embodiment, a coated and uncoated surface are at an oblique angle to each other. In another embodiment, a coated and uncoated surface are adjacent. In another embodiment, a coated and uncoated surface are nonadjacent. In another embodiment, a coated and uncoated surface are in contact with each other. In another embodiment, a coated and uncoated surface are not in contact with each other.

In other applications, one or more surfaces of an implantable device made from reticulated elastomeric matrix 10 may be coated, fused or melted to improve its attachment efficiency to attaching means, e.g., anchors or sutures, so that the attaching means does not tear-through or pull-out from the implantable device. Without being bound by any particular theory, creation of additional contact anchoring surface(s) on the implantable device, as described above, is thought to inhibit tear-through or pull-out by providing fewer voids and greater resistance.

The fusion and/or selective melting of the outer layer of elastomeric matrix 10 can be brought about in several different ways. In one embodiment, a knife or a blade used to cut a block of elastomeric matrix 10 into sizes and shapes for making final implantable devices can be heated to an elevated temperature, for example, as described in Example 7. In another embodiment, a device of desired shape and size is cut from a larger block of elastomeric matrix 10 by using a laser cutting device and, in the process, the surfaces that come into contact with the laser beam are fused. In another embodiment, a cold laser cutting device is used to cut a device of desired shape and size. In yet another embodiment, a heated mold can be used to impart the desired size and shape to the device by the process of heat compression. A slightly oversized elastomeric matrix 10, cut from a larger block, can be placed into a heated mold. The mold is closed over the cut piece to reduce its overall dimensions to the desired size and shape and fuse those surfaces in contact with the heated mold, for example, as described in Example 8. In each of the aforementioned embodiments, the processing temperature for shaping and sizing is greater than about 15° C. in one embodiment. In another embodiment, the processing temperature for shaping and sizing is in excess of about 100° C. In another embodiment, the processing temperature for shaping and sizing is in excess of about 130° C. In another embodiment, the layer(s) and/or portions of the outermost surface not being fused are protected from exposure by covering them during the fusing of the outermost surface.

The coating on the outer surface can be made from a biocompatible polymer, which can include be both biodegradable and non-biodegradable polymers. Suitable biocompatible polymers include those biocompatible polymers disclosed in the previous section. It is, of course, to be understood that that listing of materials is illustrative but not limiting. In one embodiment, surface pores are closed by applying an absorbable polymer melt coating onto a shaped elastomeric matrix. Together, the elastomeric matrix and the coating form the device. In another embodiment, surface pores are closed by applying an absorbable polymer solution coating onto a shaped elastomeric matrix to form a device. In another embodiment, the coating and the elastomeric matrix, taken together, occupy a larger volume than the uncoated elastomeric matrix alone.

The coating on elastomeric matrix 10 can be applied by, e.g., dipping or spraying a coating solution comprising a polymer or a polymer that is admixed with a pharmaceutically-active agent. In one embodiment, the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight. In another embodiment, the layer(s) and/or portions of the outermost surface not being solution-coated are protected from exposure by covering them during the solution-coating of the outermost surface. The solvent or solvent blend for the coating solution is chosen, e.g., based on the considerations discussed in the previous section (i.e., in the “Imparting Endopore Features” section).

In one embodiment, the coating on elastomeric matrix 10 may be applied by melting a film-forming coating polymer and applying the melted polymer onto the elastomeric matrix 10 by dip coating, for example, as described in Example 9. In another embodiment, the coating on elastomeric matrix 10 may be applied by melting the film-forming coating polymer and applying the melted polymer through a die, in a process such as extrusion or coextrusion, as a thin layer of melted polymer onto a mandrel formed by elastomeric matrix 10. In either of these embodiments, the melted polymer coats the outermost surface and bridges or plugs pores of that surface but does not penetrate into the interior to any significant depth. Without being bound by any particular theory, this is thought to be due to the high viscosity of the melted polymer. Thus, the reticulated nature of portions of the elastomeric matrix removed from the outermost surface, and portions of the outermost elastomeric matrix surface not in contact with the melted polymer, is maintained. Upon cooling and solidifying, the melted polymer forms a layer of solid coating on the elastomeric matrix 10. In one embodiment, the processing temperature of the melted thermoplastic coating polymer is at least about 60° C. In another embodiment, the processing temperature of the melted thermoplastic coating polymer is at least above about 90° C. In another embodiment, the processing temperature of the melted thermoplastic coating polymer is at least above about 120° C. In another embodiment, the layer(s) and/or portions of the outermost surface not being melt-coated are protected from exposure by covering them during the melt-coating of the outermost surface.

Another embodiment of the invention employs a collagen-coated composite elastomeric implantable device, as described above, configured as a sleeve extending around the implantable device. The collagen matrix sleeve can be implanted at a vascular malformation site, either adjacent to and in contact with that site. So located, the collagen matrix sleeve can be useful to help retain the elastomeric matrix 10, facilitate the formation of a tissue seal and help prevent leaks. The presence of the collagen in elastomeric matrix 10 can enhance cellular ingrowth and proliferation and improve mechanical stability, in one embodiment, by enhancing the attachment of fibroblasts to the collagen. The presence of collagen can stimulate earlier and/or more complete infiltration of the interconnected pores of elastomeric matrix 10.

Pharmaceutically-Active Agent Delivery

In another embodiment, the film-forming polymer used to coat reticulated elastomeric matrix 10 can provide a vehicle for the delivery of and/or the controlled release of a pharmaceutically-active agent, for example, a drug, such as is described in the copending applications. In another embodiment, the pharmaceutically-active agent is admixed with, covalently bonded to and/or adsorbed in or on the coating of elastomeric matrix 10 to provide a pharmaceutical composition. In another embodiment, the components, polymers and/or blends used to form the foam comprise a pharmaceutically-active agent. To form these foams, the previously described components, polymers and/or blends are admixed with the pharmaceutically-active agent prior to forming the foam or the pharmaceutically-active agent is loaded into the foam after it is formed.

In one embodiment, the coating polymer and pharmaceutically-active agent have a common solvent. This can provide a coating that is a solution. In another embodiment, the pharmaceutically-active agent can be present as a solid dispersion in a solution of the coating polymer in a solvent.

A reticulated elastomeric matrix 10 comprising a pharmaceutically-active agent may be formulated by mixing one or more pharmaceutically-active agent with the polymer used to make the foam, with the solvent or with the polymer-solvent mixture and foamed. Alternatively, a pharmaceutically-active agent can be coated onto the foam, in one embodiment, using a pharmaceutically-acceptable carrier. If melt-coating is employed, then, in another embodiment, the pharmaceutically-active agent withstands melt processing temperatures without substantial diminution of its efficacy.

Formulations comprising a pharmaceutically-active agent can be prepared by admixing, covalently bonding and/or adsorbing one or more pharmaceutically-active agents with the coating of the reticulated elastomeric matrix 10 or by incorporating the pharmaceutically-active agent into additional hydrophobic or hydrophilic coatings. The pharmaceutically-active agent may be present as a liquid, a finely divided solid or another appropriate physical form. Typically, but optionally, the matrix can include one or more conventional additives, such as diluents, carriers, excipients, stabilizers and the like.

In another embodiment, a top coating can be applied to delay release of the pharmaceutically-active agent. In another embodiment, a top coating can be used as the matrix for the delivery of a second pharmaceutically-active agent. A layered coating, comprising respective layers of fast- and slow-hydrolyzing polymer, can be used to stage release of the pharmaceutically-active agent or to control release of different pharmaceutically-active agents placed in the different layers. Polymer blends may also be used to control the release rate of different pharmaceutically-active agents or to provide a desirable balance of coating characteristics (e.g., elasticity, toughness) and drug delivery characteristics (e.g., release profile). Polymers with differing solvent solubilities can be used to build-up different polymer layers that may be used to deliver different pharmaceutically-active agents or to control the release profile of a pharmaceutically-active agents.

The amount of pharmaceutically-active agent present depends upon the particular pharmaceutically-active agent employed and medical condition being treated. In one embodiment, the pharmaceutically-active agent is present in an effective amount. In another embodiment, the amount of pharmaceutically-active agent represents from about 0.01% to about 60% of the coating by weight. In another embodiment, the amount of pharmaceutically-active agent represents from about 0.01% to about 40% of the coating by weight. In another embodiment, the amount of pharmaceutically-active agent represents from about 0.1% to about 20% of the coating by weight.

Many different pharmaceutically-active agents can be used in conjunction with the reticulated elastomeric matrix. In general, pharmaceutically-active agents that may be administered via pharmaceutical compositions of this invention include, without limitation, any therapeutic or pharmaceutically-active agent (including but not limited to nucleic acids, proteins, lipids, and carbohydrates) that possesses desirable physiologic characteristics for application to the implant site or administration via a pharmaceutical compositions of the invention. Therapeutics include, without limitation, antiinfectives such as antibiotics and antiviral agents; chemotherapeutic agents (e.g., anticancer agents); anti-rejection agents; analgesics and analgesic combinations; anti-inflammatory agents; hormones such as steroids; growth factors (including but not limited to cytokines, chemokines, and interleukins) and other naturally derived or genetically engineered proteins, polysaccharides, glycoproteins and lipoproteins. These growth factors are described in The Cellular and Molecular Basis of Bone Formation and Repair by Vicki Rosen and R. Scott Thies, published by R. G. Landes Company, hereby incorporated herein by reference. Additional therapeutics include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroids, non-steroidal anti-inflammatory agents, immunosuppressive agents, growth hormone antagonists, growth factors, dopamine agonists, radiotherapeutic agents, peptides, proteins, enzymes, extracellular matrix components, angiotensin-converting enzyme (ACE) inhibitors, free radical scavengers, chelators, antioxidants, anti polymerases, antiviral agents, photodynamic therapy agents and gene therapy agents.

Additionally, various proteins (including short chain peptides), growth agents, chemotatic agents, growth factor receptors or ceramic particles can be added to the foams during processing, adsorbed onto the surface or back-filled into the foams after the foams are made. For example, in one embodiment, the pores of the foam may be partially or completely filled with biocompatible resorbable synthetic polymers or biopolymers (such as collagen or elastin), biocompatible ceramic materials (such as hydroxyapatite), and combinations thereof, and may optionally contain materials that promote tissue growth through the device. Such tissue-growth materials include but are not limited to autograft, allograft or xenograft bone, bone marrow and morphogenic proteins. Biopolymers can also be used as conductive or chemotactic materials, or as delivery vehicles for growth factors. Examples include recombinant collagen, animal-derived collagen, elastin and hyaluronic acid. Pharmaceutically-active coatings or surface treatments could also be present on the surface of the materials. For example, bioactive peptide sequences (RGD's) could be attached to the surface to facilitate protein adsorption and subsequent cell tissue attachment.

Bioactive molecules include, without limitation, proteins, collagens (including types IV and XVIII), fibrillar collagens (including types I, II, III, V, XI), FACIT collagens (types IX, XII, XIV), other collagens (types VI, VII, XIII), short chain collagens (types VIII, X), elastin, entactin-1, fibrillin, fibronectin, fibrin, fibrinogen, fibroglycan, fibromodulin, fibulin, glypican, vitronectin, laminin, nidogen, matrilin, perlecan, heparin, heparan sulfate proteoglycans, decorin, filaggrin, keratin, syndecan, agrin, integrins, aggrecan, biglycan, bone sialoprotein, cartilage matrix protein, Cat-301 proteoglycan, CD44, cholinesterase, HB-GAM, hyaluronan, hyaluronan binding proteins, mucins, osteopontin, plasminogen, plasminogen activator inhibitors, restrictin, serglycin, tenascin, thrombospondin, tissue-type plasminogen activator, urokinase type plasminogen activator, versican, von Willebrand factor, dextran, arabinogalactan, chitosan, polyactide-glycolide, alginates, pullulan, gelatin and albumin.

Additional bioactive molecules include, without limitation, cell adhesion molecules and matricellular proteins, including those of the immunoglobulin (Ig; including monoclonal and polyclonal antibodies), cadherin, integrin, selectin, and H-CAM superfamilies. Examples include, without limitation, AMOG, CD2, CD4, CD8, C-CAM (CELL-CAM 105), cell surface galactosyltransferase, connexins, desmocollins, desmoglein, fasciclins, F11, GP Ib-IX complex, intercellular adhesion molecules, leukocyte common antigen protein tyrosine phosphate (LCA, CD45), LFA-1, LFA-3, mannose binding proteins (MBP), MTJC18, myelin associated glycoprotein (MAG), neural cell adhesion molecule (NCAM), neurofascin, neruoglian, neurotactin, netrin, PECAM-1, PH-20, semaphorin, TAG-1, VCAM-1, SPARC/osteonectin, CCN1 (CYR61), CCN2 (CTGF; Connective Tissue Growth Factor), CCN3 (NOV), CCN4 (WISP-1), CCN5 (WISP-2), CCN6 (WISP-3), occludin and claudin. Growth factors include, without limitation, BMP's (1-7), BMP-like Proteins (GFD-5, -7, -8), epidermal growth factor (EGF), erythropoietin (EPO), fibroblast growth factor (FGF), growth hormone (GH), growth hormone releasing factor (GHRF), granulocyte colony-stimulating factor (G-CSF), granulocyte-macrophage colony-stimulating factor (GM-CSF), insulin, insulin-like growth factors (IGF-I, IGF-II), insulin-like growth factor binding proteins (IGFBP), macrophage colony-stimulating factor (M-CSF), Multi-CSF (II-3), platelet-derived growth factor (PDGF), tumor growth factors (TGF-alpha, TGF-beta), tumor necrosis factor (TNF-alpha), vascular endothelial growth factors (VEGF's), angiopoietins, placenta growth factor (PIGF), interleukins, and receptor proteins or other molecules that are known to bind with the aforementioned factors. Short-chain peptides include, without limitation (designated by single letter amino acid code), RGD, EILDV, RGDS, RGES, RFDS, GRDGS, GRGS, GRGDTP and QPPRARI.

Other Post-Processing of the Reticulated Elastomeric Matrix

Elastomeric matrix 10 can undergo a further processing step or steps, in addition to reticulation and imparting endpore features, already discussed above. For example, elastomeric matrix 10 may be endoporously hydrophilized, as described above, by post treatments or by placing the elastomeric matrix in a hydrophilic environment, to render its microstructural surfaces chemically more reactive. In another embodiment, biologically useful compounds, or controlled release formulations containing them, may be attached to the endoporous surfaces for local delivery and release, embodiments which are described in the copending applications.

In another embodiment, the products made from elastomeric matrix 10 of the invention can be annealed to stabilize the structure. Annealing at elevated temperatures can promote crystallinity in semi-crystalline polyurethanes. The structural stabilization and/or additional crystallinity can provide enhanced shelf-life stability to implantable-devices made from elastomeric matrix 10. In one embodiment, annealing is carried out at temperatures in excess of about 50° C. In another embodiment, annealing is carried out at temperatures in excess of about 100° C. In another embodiment, annealing is carried out at temperatures in excess of about 125° C. In another embodiment, annealing is carried out for at least about 2 hours. In another embodiment, annealing is carried out for from about 4 to about 8 hours. In crosslinked polyurethanes, curing at elevated temperatures can also promote structural stabilization and long term shelf-life stability.

Elastomeric matrix 10 may be molded into any of a wide variety of shapes and sizes during its formation or production. The shape may be a working configuration, such as any of the shapes and configurations described in the copending applications, or the shape may be for bulk stock. Stock items may subsequently be cut, trimmed, punched or otherwise shaped for end use. The sizing and shaping can be carried out by using a blade, punch, drill or laser, for example. In each of these embodiments, the processing temperature or temperatures of the cutting tools for shaping and sizing can be greater than about 100° C. In another embodiment, the processing temperature(s) of the cutting tools for shaping and sizing can be greater than about 130° C. Finishing steps can include, in one embodiment, trimming of macrostructural surface protrusions, such as struts or the like, which can irritate biological tissues. In another embodiment, finishing steps can include heat annealing. Annealing can be carried out before or after final cutting and shaping.

Shaping and sizing can include custom shaping and sizing to match an implantable device to a specific treatment site in a specific patient, as determined by imaging or other techniques known to those in the art. In particular, one or a small number, e.g. less than about 15 in one embodiment and less than about 6 in another embodiment, of elastomeric matrices 10 can comprise an implantable device system for treating an undesired cavity, for example, a vascular malformation.

The dimensions of the shaped and sized devices made from elastomeric matrix 10 can vary depending on the particular vascular malformation treated. In one embodiment, the major dimension of a device prior to being compressed and delivered is from about 1 mm to about 100 mm. In another embodiment, the major dimension of a device prior to being compressed and delivered is from about 1 mm to about 7 mm. In another embodiment, the major dimension of a device prior to being compressed and delivered is from about 7 mm to about 10 mm. In another embodiment, the major dimension of a device prior to being compressed and delivered is from about 10 mm to about 30 mm. In another embodiment, the major dimension of a device prior to being compressed and delivered is from about 30 mm to about 100 mm. Elastomeric matrix 10 can exhibit compression set upon being compressed and transported through a delivery-device, e.g., a catheter, syringe or endoscope. In another embodiment, compression set and its standard deviation are taken into consideration when designing the pre-compression dimensions of the device.

In one embodiment, a patient is treated using an implantable device or a device system that does not, in and of itself, entirely fill the target cavity or other site in which the device system resides, in reference to the volume defined within the entrance to the site. In one embodiment, the implantable device or device system does not entirely fill the target cavity or other site in which the implant system resides even after the elastomeric matrix pores are occupied by biological fluids or tissue. In another embodiment, the fully expanded in situ volume of the implantable device or device system is at least 1% less than the volume of the site. In another embodiment, the fully expanded in situ volume of the implantable device or device system is at least 15% less than the volume of the site. In another embodiment, the fully expanded in situ volume of the implantable device or device system is at least 30% less than the volume of the site.

The implantable device or device system may comprise one or more elastomeric matrices 10 that occupy a central location in the cavity. The implantable device or device system may comprise one or more elastomeric matrices 10 that are located at an entrance or portal to the cavity. In another embodiment, the implantable device or device system includes one or more flexible, possibly sheet-like, elastomeric matrices 10. In another embodiment, such elastomeric matrices, aided by suitable hydrodynamics at the site of implantation, migrate to lie adjacent to the cavity wall.

In another embodiment, the fully-expanded in situ volume of the implantable device or device system is from about 1% to about 40% larger than the volume of the cavity. In another embodiment, the fully-expanded in situ volume of the implantable device or device system is from about 5% to about 25% larger than the volume of the cavity. In another embodiment, the ratio of implantable device volume to the volume occupied by the vascular malformation is from about 70% to about 90%. In another embodiment, the ratio of implantable device volume to the volume occupied by the vascular malformation is from about 90% to about 100%. In another embodiment, the ratio of implantable device volume to the volume occupied by the vascular malformation is from about 90% to less than about 100%. In another embodiment, the ratio of implantable device volume to the volume occupied by the vascular malformation is from about 100% to about 140%.

Biodurable reticulated elastomeric matrices 10, or an implantable device system comprising such matrices, can be sterilized by any method known to the art including gamma irradiation, autoclaving, ethylene oxide sterilization, infrared irradiation and electron beam irradiation. In one embodiment, biodurable elastomers used to fabricate elastomeric matrix 10 tolerate such sterilization without loss of useful physical and mechanical properties. The use of gamma irradiation can potentially provide additional crosslinking to enhance the performance of the device.

In one embodiment, the sterilized products may be packaged in sterile packages of paper, polymer or other suitable material. In another embodiment, within such packages, elastomeric matrix 10 is compressed within a retaining member to facilitate its loading into a delivery-device, such as a catheter or endoscope, in a compressed configuration. In another embodiment, elastomeric matrix 10 comprises an elastomer with a compression set enabling it to expand to a substantial proportion of its pre-compressed volume, e.g., at 25° C., to at least 50% of its pre-compressed volume. In another embodiment, expansion occurs after elastomeric matrix 10 remains compressed in such a package for typical commercial storage and distribution times, which will commonly exceed 3 months and may be up to 1 or 5 years from manufacture to use.

Radio-Opacity

In one embodiment, implantable device can be rendered radio-opaque to facilitate in vivo imaging, for example, by adhering to, covalently bonding to and/or incorporating into the elastomeric matrix itself particles of a radio-opaque material. Radio-opaque materials include titanium, tantalum, tungsten, barium sulfate or other suitable material known to those skilled in the art.

Implantable Device Uses

Reticulated elastomeric matrix 10, and implantable device systems incorporating the same, can be used as described in the copending applications. In one non-limiting example, one or more reticulated elastomeric matrix 10 is selected for a given site. Each, in turn, is compressed and loaded into a delivery-device, such as a catheter, endoscope, syringe or the like. The delivery-device is snaked through the vasculature or other vessel system of the intended patient host and the reticulated elastomeric matrix 10 is released into the target site. Once released at the site, reticulated elastomeric matrix 10 expands resiliently to about its original, relaxed size and shape subject, of course, to its compression set limitation and any desired flexing, draping or other conformation to the site anatomy that the implantable device may adopt.

Without being bound by any particular theory, it is thought that, in situ, hydrodynamics such as pulsatile blood pressure may, with suitably shaped reticulated elastomeric matrices 10, e.g., cause the elastomeric matrix to migrate to the periphery of the site, e.g., close to the wall. When the reticulated elastomeric matrix 10 is placed in or carried to a conduit, e.g., a lumen or vessel through which body fluid passes, it will provide an immediate resistance to the flow of body fluid such as blood. This will be associated with an inflammatory response and the activation of a coagulation cascade leading to formation of a clot, owing to a thrombotic response. Thus, local turbulence and stagnation points induced by the implantable device surface may lead to platelet activation, coagulation, thrombin formation and clotting of blood.

In one embodiment, cellular entities such as fibroblasts and tissues can invade and grow into reticulated elastomeric matrix 10. In due course, such ingrowth can extend into the interior pores 20 and interstices of the inserted reticulated elastomeric matrix 10. Eventually, elastomeric matrix 10 can become substantially filled with proliferating cellular ingrowth that provides a mass that can occupy the site or the void spaces in it. The types of tissue ingrowth possible include, but are not limited to, fibrous tissues and endothelial tissues.

In another embodiment, the implantable device or device system causes cellular ingrowth and proliferation throughout the site, throughout the site boundary, or through some of the exposed surfaces, thereby sealing the site. Over time, this induced fibrovascular entity resulting from tissue ingrowth can cause the implantable device to be incorporated into the conduit. Tissue ingrowth can lead to very effective resistance to migration of the implantable device over time. It may also prevent recanalization of the conduit. In another embodiment, the tissue ingrowth is scar tissue which can be long-lasting, innocuous and/or mechanically stable. In another embodiment, over the course of time, for example for 2 weeks to 3 months to 1 year, implanted reticulated elastomeric matrix 10 becomes completely filled and/or encapsulated by tissue, fibrous tissue, scar tissue or the like.

The features of the implantable device, its functionality and interaction with conduits, lumens and cavities in the body, as indicated above, can be useful in treating a number of arteriovenous malformations (“AVM”) or other vascular abnormalities. These include AVMs, anomalies of feeding and draining veins, arteriovenous fistulas, e.g., anomalies of large arteriovenous connections, abdominal aortic aneurysm endograft endoleaks (e.g., inferior mesenteric arteries and lumbar arteries associated with the development of Type II endoleaks in endograft patients), gastrointestinal hemorrhage, pseudoaneurysms, varicocele occlusion and female tubular occlusion.

In another embodiment, for aneurysm treatment, a reticulated elastomeric matrix 10 is placed between the site wall and a graft element that is inserted to treat the aneurysm. Typically, when a graft element is used alone to treat an aneurysm, it becomes partially surrounded by ingrown tissue, which may provide a site where an aneurysm can re-form or a secondary aneurysm can form. In some cases, even after the graft is implanted to treat the aneurysm, undesirable occlusions, fluid entrapments or fluid pools may occur, thereby reducing the efficacy of the implanted graft. By employing the inventive reticulated elastomeric matrix 10, as described herein, it is thought, without being bound by any particular theory, that such occlusions, fluid entrapments or fluid pools can be avoided and that the treated site may become completely ingrown with tissue, including fibrous tissue and/or endothelial tissues, secured against blood leakage or risk of hemorrhage, and effectively shrunk. In one embodiment, the implantable device may be immobilized by fibrous encapsulation and the site may even become sealed, more or less permanently.

In one embodiment, the implantation site and the surrounding conduits can be imaged by arterial angiograms. In another embodiment, they can also be imaged to map or model the three-dimensional topography of the intended site to facilitate the choice of reticulated elastomeric matrix 10. The size and shape of the implantable device can then be estimated before it is delivered to the targeted site. Alternatively, reticulated elastomeric matrix 10 can be custom-fabricated to fit or to be accommodated in the intended site using suitable imaging technology, e.g., magnetic resonance imaging (MRI), computerized tomography scanning (CT Scan), x-ray imaging employing contrast material or ultrasound. Other suitable imaging methods will be known to those skilled in the art.

In a further embodiment, the implantable devices disclosed herein can be used as a drug delivery vehicle. For example, the biodurable solid phase 12 can be mixed, covalently bonded to and/or adsorbed in a therapeutic agent. Any of a variety of therapeutic agents can be delivered by the implantable device, for example, those therapeutic agents previously disclosed herein.

### EXAMPLES

The following examples further illustrate certain embodiments of the present invention. These examples are provided solely for illustrative purposes and in no way limit the scope of the present invention.

### Example 1

Fabrication of a Polycarbonate Polyurethane Matrix by Sacrificial Molding

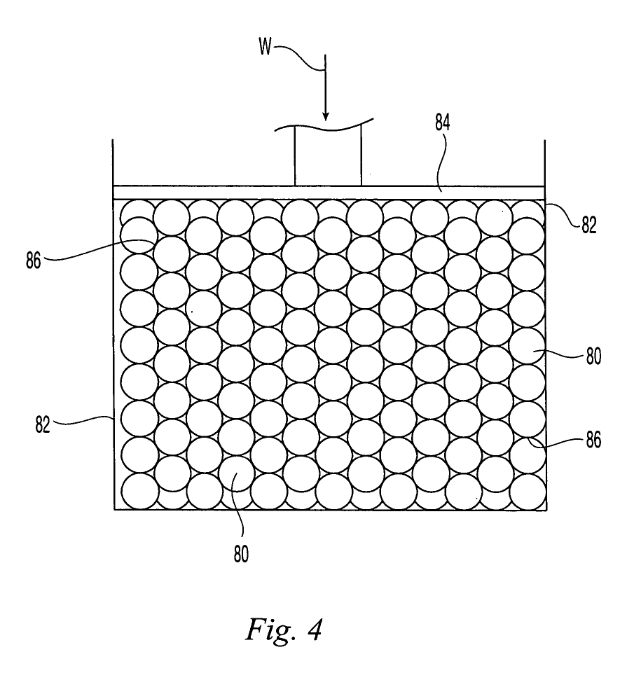

As shown in FIG. 4, a substrate was prepared by fusing together particles 80, e.g., under modest temperature and pressure, spherical waxy particles 80 formed of e.g., VYBAR® 260 hydrocarbon polymer obtained from Baker Petrolite (Sugar Land, Tex.). Particles 80 were screened to a relatively narrow diameter distribution, about 3 mm to about 5 mm in diameter, before use. About 20 mL of the screened particles were poured into a transparent 100 mL polypropylene disposable beaker with a perforated bottom, i.e., vessel 82, to provide a compact three-dimensional mass with significant height in the beaker. The beaker was placed into a sealant sleeve attached to a buchner flask which was, in turn, attached to a low-pressure source.

A pressure of about 3-5 psi (about 2,100-3,500 kg/m²) was applied to wax particles 80 by employing a weight W supported on a load-spreading plate 84 resting on the wax particles so as to apply compressive force on the particles. The beaker was warmed to a temperature of from about 50° C. to about 55° C. The wax particles were closely packed in the beaker, contacting each other at about 5 to 8 contact points 86 per particle. The compression was continued until flattening of the particle interfaces occurred, which was be determined by visually observing particle flattening against the transparent beaker wall, by inverting the beaker and noting that no particles fall from the mass, or by both of these methods. Care was taken to avoid over-compression, thus ensuing that adequate volume of interstitial passageways remained between the particles.

A 10% by weight of grade 80A BIONATE® polycarbonate polyurethane solution in THF was prepared by tumbling and agitating the BIONATE® pellets in the THF using a rotary spider turning at 5 rpm over a 3 day period. The solution was made in a sealed container to minimize solvent loss.

About 60 mL of the 10% polymer solution was poured onto the top layer of the wax particles. A reduced pressure of about 5 inches of mercury was applied to the buchner flask. As soon as the polymer solution was drawn down into the wax particles, an additional 20 mL of particles was poured onto the upper layer of the scaffold and a load-spreading plate slightly smaller than the inside diameter of the beaker was applied to the top of the particles. A pressure of about 3-5 psi (about 2,100-3,500 kg/m²) was then applied to the plate. Application of the reduced pressure to the buchner flask was halted as soon as air was heard hissing through the particles, the compression was removed, and the resulting “plug” was then allowed to set for about 1 hour. After this period, the beaker was inverted and any excess particles removed from the plug.

The plug was placed into a stainless steel basket in an air current for about 16 hours to remove the residual THF, thereby providing a solid block with the interstices between the polycarbonate polyurethane containing the waxy particles. When dry, the plug was distorted to loosen any wax particles not imbedded in the polymer, placed into a stainless steel basket, and the basket was placed into an oven maintained at about 85° C. to 90° C. for about 1 hour to melt out the wax. If required, the plug may be compressed to help displace excess liquid wax. The porous polymer block was washed repeatedly in hexane to remove residual wax and allowed to air dry.

The average pore diameter of the elastomeric matrix, as determined from scanning electron micrograph (“SEM”) observations, was from about 200 μm to about 500 μm. The elastomeric matrix appeared to have a reticulated structure without any or, at most, only a few residual cell walls. This feature provides extremely favorable potential for cellular ingrowth and proliferation.

Cylinders measuring 10, 15 and 20 mm in diameter and 5, 8 and 10 mm in length and cubes with 10 mm sides were cut from the reticulated material block to form prototype devices.

### Example 2

Fabrication of a Polycarbonate Polyurethane Matrix by Sacrificial Molding

Example 1 is thrice repeated, each time employing smaller particles, i.e., having average sizes of 1.5, 1 and 0.5 mm, respectively. Results comparable to Example 1 are obtained in each case.

### Example 3

Fabrication of a Polycarbonate Polyurethane Matrix by Sacrificial Molding Alternative Method

A solution of BIONATE® 80A in THF was made according to Example 1 except that its concentration was 7% by weight of the polycarbonate polyurethane polymer. As also described in Example 1, VYBAR 260 hydrocarbon polymer particles were used except that the particles were screened to a relatively narrow diameter distribution, about 1 mm to about 2 mm in diameter, before use.

As described in Example 1, about 20 mL of the 7% polymer solution was poured onto the top layer of the wax particles. However, in this example, the wax particles in the beaker were neither heated nor compressed before being contacted by the solution. A reduced pressure of about 5 inches of mercury was applied to the buchner flask. As soon as the polymer solution was drawn down into the wax particles, an additional 20 mL of particles was poured onto the upper layer of the scaffold and a load-spreading plate slightly smaller than the inside diameter of the beaker was applied to the top of the particles. A pressure of about 3-5 psi (about 2,100-3,500 kg/m²) was then applied to the plate. Application of the reduced pressure to the buchner flask was halted as soon as air was heard hissing through the particles, the compression was removed, and the resulting “plug” was then allowed to set for about 1 hour. After this period, the beaker was inverted and any excess particles removed from the plug. Thereafter, the THF and wax were removed as described in Example 1 and the porous polymer block was washed repeatedly in hexane to remove residual wax and allowed to air dry.

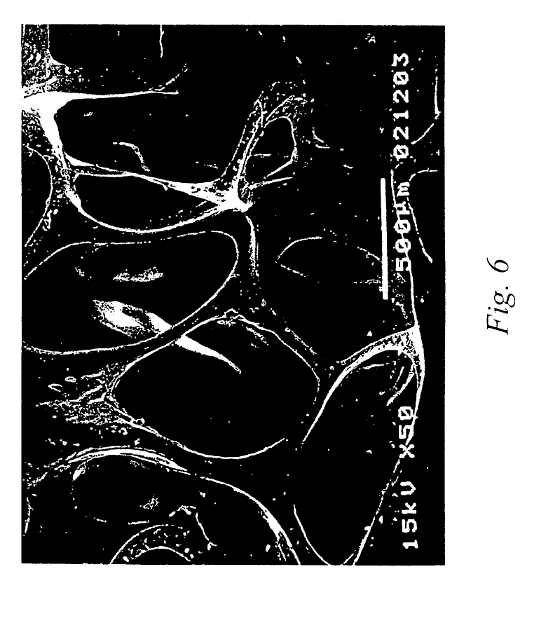

The polymer block, as evident from the representative SEM image of that block in FIG. 6, appeared to have a reticulated structure without any or, at most, only a few residual cell walls. It should be noted that the SEM image in FIG. 6 displays many of the same features, e.g., reticulated solid phase 12, continuous interconnected void phase 14, a multiplicity of struts 16 that extend between and interconnect a number of intersections 18, and a multitude of pores 20, that are depicted schematically in FIG. 1. The reticulated nature of the polymer block provides extremely favorable potential for cellular ingrowth and proliferation.

The density of the reticulated elastomeric matrix material was determined by accurately weighing a known volume of material, here 13.75 cc, and dividing the weight by the volume to obtain a density of 0.045 gm/cc or 2.8 lbs/ft³. The void volume was determined to be about 96%.

Tensile tests were conducted on samples with dimensions of 50 mm long×25 mm wide×12.5 mm thick. The gauge length was 25 mm and the cross-head speed was 25 mm/minute. The tensile strength of the reticulated elastomeric matrix material was determined to be 19.3 psi (13,510 kg/m²) and the elongation to break was 466%.

Cylinders measuring 10, 15 and 20 mm in diameter and 5, 8 and 10 mm in length and cubes with 10 mm sides were cut from the reticulated material block to form prototype devices.

### Example 4

Fabrication of a Polycarbonate Polyurethane Matrix by Sacrificial Molding Using Co-solvents

Particles of VYBAR 260 branched hydrocarbon polymer, obtained from Baker Petrolite, were melted and extruded at a temperature of from 90° C. to 105° C. through a 0.75 inch (19 mm) diameter spinning nozzle. The extrudate passed into a beaker filled with a mixture of 90 wt. % isopropanol/10 wt. % water maintained at a temperature of from 15° C. to 30° C. The height of the surface of the mixture was adjusted such that the top of the mixture was 22 inches (560 mm) below the bottom of the nozzle. The solidified beads were collected by passing the bead/mixture slurry through a sieve of mesh size smaller than #25 (710 μm). The sieve containing the beads was placed in a HEPA filtered air stream to dry the beads for at least 4 hours. The dried beads were again sieved. Twice-sieved beads in the range of from 1.7 mm to 4 mm in diameter were used.

Co-solvents were used to form a polycarbonate polyurethane/tantalum solution. A 5 wt. % BIONATE 80A polycarbonate polyurethane, together with tantalum powder weighing 10% by weight of the BIONATE or 0.5 wt. % overall, solution in a 97 wt. % THF/3 wt. % DMF mixture was prepared by tumbling and agitating the ingredients using a rotary spider turning at 5 rpm over a 3 day period. The solution was made in a sealed container to minimize solvent loss. The 99.9% pure tantalum powder of 325 mesh size was obtained from the Aldrich Chemical Co. (Milwaukee, Wis.) Thereafter, the mixture was heated in an oven at 60° C. for 24 hours then cooled to about 25° C. The solution viscosity was determined to be 310 centipoise at about 25° C.

About 500 mL of the above-described twice-sieved beads were poured into a transparent 1 L polypropylene disposable beaker with a perforated bottom. The bead-filled beaker was placed into a vacuum chamber, the pressure was reduced using a vacuum pump, and the beads were covered with 125 mL of the above-described 5 wt. % BIONATE polymer solution while maintaining the chamber pressure at from 5 to10 in. Hg. The vacuum pump was disconnected as soon as the solution sank below the top surface of the beads. The beads were covered with about an additional 100 mL of twice-sieved beads and gentle pressure was applied to the top of the bead layer using the base of a clean beaker.

Thereafter, the solution-containing beads are placed onto a drying rack under a fume-hood for about a 3-4 hour period to allow the THFIDMF mixture to evaporate. Then, the beads are dried under reduced pressure at about 40° C. for a 24-48 hour period to remove any residual solvent. A plug of polymer and wax is obtained. The plug can optionally be washed in water and kept under reduced pressure at about 40° C. for an additional 12 hour period to remove the water and any residual solvent, if required.

After drying, the plug is gently mechanically distorted to loosen any wax particles not imbedded in the polymer, which are removed. Thereafter, the plug is placed onto a stainless steel rack and placed over a tray. The assembly is placed into an oven maintained at from about 80° C. to 85° C. to for about 1-3 hours to melt the wax and allow it to flow from the plug into the tray. If required, the plug is compressed to help displace liquified wax from the plug. The resulting elastomeric matrix is washed repeatedly in hexane, replacing the hexane wash with fresh hexane at least two times. Thereafter, the elastomeric matrix undergoes additional washing for about 2 hours in 75-80° C. heptane to remove any residual wax. The elastomeric matrix is allowed to air dry at about 25° C.

The elastomeric matrix appears to have a reticulated structure with few or no residual cell walls. This aspect is favorable for promoting cellular ingrowth and proliferation.

### Example 5

Fabrication of a CHRONOFLEX® Polvurethane Matrix by Sacrificial Molding

Example 3 is repeated employing CHRONOFLEX® C polyurethane elastomer in place of BIONATE® polycarbonate polyurethane and using N-methyl-2-pyrrolidone in place of THF. Results comparable to Example 3 are obtained.

### Example 6

Determination of Tissue Ingrowth

In order to determine the extent of cellular ingrowth and proliferation using a reticulated elastomeric matrix implantable device of the invention, surgery was performed in which such reticulated implantable devices were placed in the subcutaneous tissue of Sprague-Dawley rats.

Eight Sprague-Dawley rats weighing from about 375 g to about 425 g each were given access to food and water ad libitum before anesthesia was induced with an intraperitoneal injection of 60 mg/kg sodium pentobarbital.

After anesthesia, the animals were placed on a heating pad and maintained at a temperature of 37° C. for the entire procedure and immediate recovery period. With the animals in the supine position, a small midline abdominal wall incision was made with a number 15 scalpel. The skin and subcutaneous tissue was incised, and superficial fascia and muscle layers were separated from subcutaneous tissue with blunt dissection. One cylindrical polyurethane reticulated elastomeric matrix implantable device, made according to Example 3 and measuring about 5 mm in diameter and 8 mm in length, was then inserted into the abdominal subcutaneous pocket of each animal. The skin was closed with permanent sutures. The animals were returned to their cages and allowed to recover.

The animals were given access to food and water ad libitum for the next 14 days, then the implantable devices with skin and muscle tissue was collected from the abdominal wall. At the end of 14 days, each animal was euthanized. Anesthesia was induced with an intraperitoneal injection of 60 mg/kg sodium pentobarbital and the animals were killed by carbon dioxide. The previous incision was exposed. The abdominal wall segment containing the implantable device was removed. For each animal, the implantable device and the full thickness abdominal wall was placed into formalin for preservation.

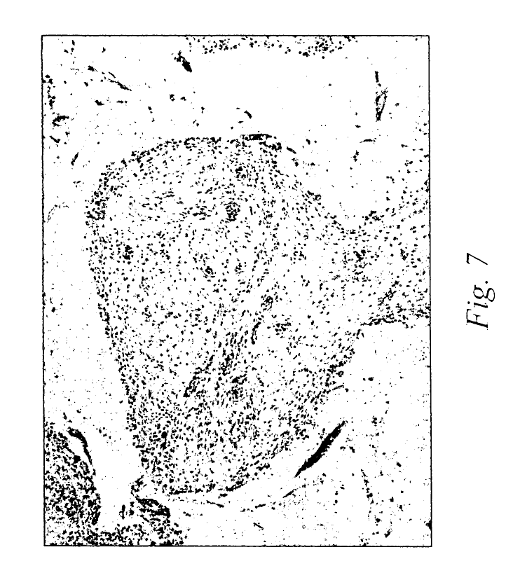

Histopathology evaluation of the implantable device within the abdominal wall was performed by conventional H&E staining. From the examination of the histology slides, FIG. 7 providing an example, the implantable device demonstrates evidence of fibrovascular ingrowth, myxoid stroma, new collagen fiber formation and early inflammatory cell response consistent with surgical implant procedure. The implantable device supported tissue ingrowth and demonstrated its capability and potential for permanent tissue replacement, cavity or blood vessel obliteration and tissue augmentation.

### Example 7

Implantable Device with Selectively Non-Porous Surface

A piece of reticulated material made according to Example 3 is used. A heated blade with a knife-edge is used to cut a cylinder 10 mm in diameter and 15 mm in length from the piece. The blade temperature is above 130° C. The surfaces of the piece in contact with the heated blade appear to be fused and non-porous from contact with the heated blade. Those surfaces of the piece that are intended to remain porous, i.e., not to fuse, are not exposed to the heated blade.

### Example 8

Implantable Device with Selectively Non-Porous Surface

A slightly oversized piece of reticulated material made according to Example 3 is used. The slightly oversized piece is placed into a mold heated to a temperature of above 130° C. The mold is then closed over the piece to reduce the overall dimensions to the desired size. Upon removing the piece from the mold, the surfaces of the piece in contact with the mold appear to be fused and non-porous from contact with the mold. Those surfaces of the piece that are intended to remain porous, i.e., not to fuse, are protected from exposure to the heated mold. A heated blade with a knife-edge is used to cut from the piece a cylinder 10 mm in diameter and 15 mm length.

### Example 9

Dip-Coated Implantable Device with Selectively Non-Porous Surface

A piece of reticulated material made according to Example 3 is used. A coating of copolymer containing 90 mole % PGA and 10 mole % PLA is applied to the outer surface as follows. The PGA/PLA copolymer is melted in an extruder at 205° C. and the piece is dipped into the melt to coat it. Those surfaces of the piece that are to remain porous, i.e., not to be coated by the melt, are covered to protect them and not exposed to the melt. Upon removal, the melt solidifies and forms a thin non-porous coating layer on the surfaces of the piece with which it comes in contact.

### Example 10

Fabrication of a Collagen-Coated Elastomeric Matrix

Collagen, obtained by extraction from bovine hide, is washed and chopped into fibrils. A 1% by weight collagen aqueous slurry is made by vigorously stirring the collagen and water and adding inorganic acid to a pH of about 3.5.

A reticulated polyurethane matrix prepared according to Example 1 is cut into a piece measuring 60 mm by 60 mm by 2 mm. The piece is placed in a shallow tray and the collagen slurry is poured over it so that the piece is completely immersed in the slurry, and the tray is optionally shaken. If necessary, excess slurry is decanted from the piece and the slurry-impregnated piece is placed on a plastic tray, which is placed on a lyophilizer tray held at 10° C. The lyophilizer tray temperature is dropped from 10° C. to −35° C. at a cooling rate of about 1° C./minute and the pressure within the lyophilizer is reduced to about 75 millitorr. After holding at −35° C. for 8 hours, the temperature of the tray is raised at a rate of about 1° C./hour to 10° C. and then at a rate of about 2.5° C./hour until a temperature of 25° C. is reached. During lyophilization, the water sublimes out of the frozen collagen slurry leaving a porous collagen matrix deposited within the pores of the reticulated polyurethane matrix piece. The pressure is returned to 1 atmosphere.

Optionally, the porous collagen-coated polyurethane matrix piece is subjected to further heat treatment at about 110° C. for about 24 hours in a current of nitrogen gas to crosslink the collagen, thereby providing additional structural integrity.

### Example 11

Fabrication of Collagen-Coated Elastomeric Matrix Tubes

A cylindrical piece of reticulated polyurethane matrix, prepared according to Example 3, measuring 10 mm in diameter and 30 mm in length is placed into a cylindrical plastic mold 50 mm in diameter and 100 mm in length. Following the process described in Example 10, an aqueous collagen slurry is poured into the mold and completely immerses the cylindrical piece of reticulated polyurethane matrix.

The slurry-containing mold is cooled as in Example 10 and placed under reduced pressure. Water is removed by sublimation as in Example 10 and, upon removal from the mold, a porous cylindrical plug is formed. The cylindrical collagen-coated elastomer plug can, optionally, be crosslinked by heat treatment, as described in Example 10. A hole measuring 5 mm in diameter is bored through the center of the plug to make a tube or hollow cylinder.

Where the tube is to be employed for treating a vascular malformation, e.g., an aneurysm, its outer diameter is selected to substantially match the inner diameter of the blood-carrying vessel and its length is selected to overlap the mouth of the aneurysm.

### Example 12

Fabrication of a Crosslinked Reticulated Polyurethane Matrix

Two aromatic isocyanates, RUBINATE® 9433 and RUBINATE 9258 (each from Huntsman; each comprising a mixture of 4,4′-MDI and 2,4′-MDI), were used as the isocyanate component. RUBINATE 9433 contains about 65% by weight 4,4′-MDI, about 35% by weight 2,4′-MDI and has an isocyanate functionality of about 2.01. RUBINATE 9258 contains about 68% by weight 4,4′-MDI, about 32% by weight 2,4′-MDI and has an isocyanate functionality of about 2.33. A modified 1,6-hexanediol carbonate (PESX-619, Hodogaya Chemical, Japan), i.e., a diol, with a molecular weight of about 2,000 Daltons was used as the polyol component. Each of these ingredients is a liquid at 25° C. The crosslinker used was glycerol, which is tri-functional. Water was used as the blowing agent. The gelling catalyst was dibutyltin dilaurate (DABCO T-12, supplied by Air Products). The blowing catalyst was the tertiary amine 33% triethylenediamine in dipropylene glycol (DABCO 33LV supplied by Air Products). A silicone-based surfactant was used (TEGOSTAB® BF 2370, supplied by Goldschmidt). The cell-opener was ORTEGOL® 501 (supplied by Goldschmidt). The proportions of the components that were used is given in Table 2.

TABLE 2

| Ingredient | Parts by Weight |
|---|---|
| Polyol Component | 100 |
| Isocyanate Component | |
| RUBINATE 9433 | 60.0 |
| RUBINATE 9258 | 17.2 |
| Isocyanate Index | 1.03 |
| Crosslinker | 2.5 |
| Water | 3.4 |
| Gelling Catalyst | 0.12 |
| Blowing Catalyst | 0.4 |
| Surfactant | 1.0 |
| Cell Opener | 0.4 |

The one-shot approach was used to make the foam. In this technique, all ingredients, except for the isocyanate component, were admixed in a beaker at 25° C. The isocyanate component was then added with high-speed stirring. The foaming mix was then poured into a cardboard form, allowed to rise, and then post-cured for 4 hours at 100° C. The foaming profile was as follows: mixing time of 10 sec., cream time of 15 sec., rise time of 28 sec., and tack-free time of 100 sec.

The average pore diameter of the foam, as observed by optical microscopy, was between 300 and 400 μm.

The following foam testing was carried out in accordance with ASTM D3574. Density was measured with specimens measuring 50 mm×50 mm×25 mm. The density was calculated by dividing the weight of the sample by the volume of the specimen; a value of 2.5 lbs/ft³ (0.040 g/cc) was obtained.

Tensile tests were conducted on samples that were cut both parallel and perpendicular to the direction of foam rise. The dog-bone shaped tensile specimens were cut from blocks of foam each about 12.5 mm thick, about 25.4 mm wide and about 140 mm long. Tensile properties (strength and elongation at break) were measured using an INSTRON Universal Testing Instrument Model 1122 with a cross-head speed of 19.6 inches/minute (500 mm/min). The tensile strength, measured in two orthogonal directions with respect to foam rise, ranged from about 40 psi (28,000 kg/m²) to about 70 psi (49,000 kg/m²). The elongation to break was approximately 76% irrespective of direction.

Compressive strengths of the foam were measured with specimens measuring 50 mm×50 mm×25 mm. The tests were conducted using an INSTRON Universal Testing Instrument Model 1122 with a cross-head speed of 0.4 inches /minute (10 mm/min). The compressive strength at 50% and 75% compression was about 42 psi (29,400 kg/m²) and about 132 psi (92,400 kg/m²), respectively.

Tear resistance strength of the foam was measured with specimens measuring approximately 152 mm×25 mm×12.7 mm. A 40 mm cut was made on one side of each specimen. The tear strength was measured using an INSTRON Universal Testing Instrument Model 1122 with a cross-head speed of 19.6 inches/minute (500 mm/min). The tear strength was determined to be about 2.3 lbs/inch (about 411 g/cm).

In the subsequent reticulation procedure, a block of foam is placed into a pressure chamber, the doors of the chamber are closed and an airtight seal is maintained. The pressure is reduced to remove substantially all of the air in the chamber. A combustible ratio of hydrogen to oxygen gas is charged into the chamber. The gas in the chamber is then ignited by a spark plug. The ignition explodes the gases within the foam cell structure. This explosion blows out many of the foam cell windows, thereby creating a reticulated elastomeric matrix structure.

### Example 13

Fabrication of a Crosslinked Reticulated Polyurethane Matrix

Chemical reticulation of the unreticulated foam of Example 12 is carried out by immersing the foam in a 30% by weight aqueous solution sodium hydroxide for 2 weeks at 25° C. Then, the sample is washed repeatedly with water and dried for 24 hours in an oven at 100° C. The resulting sample is reticulated.

### Example 14

Fabrication of a Crosslinked Reticulated Polyurethane Matrix

The isocyanate component was RUBINATE 9258, as described in Example 12. The polyol component was 1,6-hexanediol carbonate (PCDN-980R, Hodogaya Chemical), with a molecular weight of about 2,000 Daltons. This polyol was a solid at 25° C. while the isocyanate was a liquid at this temperature. Water was used as the blowing agent. The gelling catalyst, blowing catalyst, surfactant and cell opener of Example 12 were used. The proportions of the components used are described in Table 3.

TABLE 3

| Ingredient | Parts by Weight |
|---|---|
| Polyol Component | 100 |
| Isocyanate Component | 53.8 |
| Isocyanate Index | 1.00 |
| Water | 2.82 |
| Gelling Catalyst | 0.03 |
| Blowing Catalyst | 0.3 |
| Surfactant | 2.16 |
| Cell Opener | 0.48 |
| Viscosity Modifier | 5.76 |

The polyol component was preheated to 80° C. then mixed with the isocyanate component, a viscosity modifier (propylene carbonate, which served as a viscosity depressant for this formulation), surfactant and cell opener to form a viscous liquid. Then, a mixture of water, gelling catalyst and blowing catalyst was added under vigorous mixing. The foaming mix was then poured into a cardboard form, allowed to rise, and then post-cured for 4 hours at 100° C. The foaming profile was as follows: mixing time of 10 sec., cream time of 15 sec., rise time of 60 sec., and tack-free time of 120 sec.

The density, tensile properties, and compressive strength of the foam were determined as described in Example 12. The density of the foam was 2.5 lbs/ft³ (0.040 g/cc). The tensile strength, measured in two orthogonal directions with respect to foam rise, ranged from about 28 psi (about 19,600 kg/m²) to about 43 psi (about 30,100 kg/m²). The elongation to break was approximately 230% irrespective of direction. The compressive strength at 50% and 75% compression was about 17 psi (about 11,900 kg/m²) and about 34 psi (about 23,800 kg/m²), respectively.

The foam is reticulated by the procedure described in Example 12.

### Example 15

Fabrication of a Crosslinked Polyurethane Matrix

The aromatic isocyanate RUBINATE 9258 was used as the isocyanate component. RUBINATE 9258 is a liquid at 25° C. A polyol,1,6-hexamethylene polycarbonate (Desmophen LS 2391, Bayer Polymers), i.e., a diol, with a molecular weight of about 2,000 Daltons was used as the polyol component and was a solid at 25° C. Distilled water was used as the blowing agent. The blowing catalyst used was the tertiary amine DABCO 33LV. TEGOSTAB® BF 2370 was used as the silicone-based surfactant. ORTEGOL® 501 was used as the cell-opener. The viscosity modifier propylene carbonate (supplied by Sigma-Aldrich) was present to reduce the viscosity. The proportions of the components that were used is given in Table 4.

TABLE 4

| Ingredient | Parts by Weight |
|---|---|
| Polyol Component | 100 |
| Viscosity Modifier | 5.76 |
| Surfactant | 2.16 |
| Cell Opener | 0.48 |
| Isocyanate Component | 53.8 |
| Isocyanate Index | 1.00 |
| Distilled Water | 2.82 |
| Blowing Catalyst | 0.44 |

The polyol component was liquefied at 70° C. in a recirculating-air oven, and 150 g thereof was weighed out into a polyethylene cup. 8.7 g of viscosity modifier was added to the polyol component to reduce the viscosity and the ingredients were mixed at 3100 rpm for 15 seconds with the mixing shaft of a drill mixer. 3.3 g of surfactant was added and the ingredients were mixed as described above for 15 seconds. Thereafter, 0.75 g of cell opener was added and the ingredients were mixed as described above for 15 seconds. 80.9 g of isocyanate component was added and the ingredients were mixed for 60±10 seconds to form “system A.”

4.2 g of distilled water was mixed with 0.66 g of blowing catalyst in a small plastic cup for 60 seconds with a glass rod to form “System B.”

System B was poured into System A as quickly as possible while avoiding spillage. The ingredients were mixed vigorously with the drill mixer as described above for 10 seconds then poured into a 22.9 cm×20.3 cm×12.7 cm (9 in.×8 in.×5 in.) cardboard box with its inside surfaces covered by aluminum foil. The foaming profile was as follows: 10 seconds mixing time, 18 seconds cream time, and 85 seconds rise time.

2 minutes after the beginning of foaming, i.e., the time when Systems A and B were combined, the foam was place into a recirculating-air oven maintained at 100-105° C. for curing for 1 hour. Thereafter, the foam was removed from the oven and cooled for 15 minutes at about 25° C. The skin was removed from each side using a band saw and hand pressure was applied to each side of the foam to open the cell windows. The foam was replaced into the recirculating-air oven and postcured at 100-105° C. for additional 5 hours.

The average pore diameter of the foam, as determined from optical microscopy observations, was from about 150 μm to about 450 μm.

The following foam testing was carried out according to ASTM D3574. Density was measured using specimens of dimensions 50 mm×50 mm×25 mm. The density was calculated by dividing the weight of the sample by the volume of the specimen. A density value of 2.5 lbs/ft³ (0.040 g/cc) was obtained.

Tensile tests were conducted on samples that were cut either parallel or perpendicular to the direction of foam rise. The dog-bone shaped tensile specimens were cut from a block of foam. Each block measured about 12.5 mm thick, about 25.4 mm wide and about 140 mm long. Tensile properties (tensile strength and elongation at break) were measured using an INSTRON Universal Testing Instrument Model 1122 with a cross-head speed of 19.6 inches/minute (500 mm/min). The average tensile strength, determined by combining the measurements from the two orthogonal directions with respect to foam rise, was 24.64±2.35 psi (17,250±1,650 kg/m²). The elongation to break was determined to be 215±12%.

Compressive tests were conducted using specimens measuring 50 mm×50 mm×25 mm. The tests were conducted using an INSTRON Universal Testing Instrument Model 1122 with a cross-head speed of 0.4 inches /minute (10 mm/min). The compressive strength at 50% compression was determined to be 12±3 psi (8,400±2,100 kg/m²). The compression set, after subjecting the sample to 50% compression for 22 hours at 40° C. then releasing the compressive stress, was determined to be about 2%.

The tear resistance strength of the foam was determined using specimens measuring approximately 152 mm long×25 mm wide×12.7 mm thick. A 40 mm long cut in the long direction of each specimen was made through the specimen thickness, beginning at the center of one 25 mm wide side. The tear strength was measured using an INSTRON Universal Testing Instrument Model 1122 with a cross-head speed of 19.6 inches/minute (500 mm/min). The tear strength was determined to be 2.9±0.1 lbs/inch (1.32±0.05 kg/cm).

The pore structure and its inter-connectivity was characterized using a Liquid Extrusion Porosimeter (Porous Materials, Inc., Ithaca, N.Y.). In this test, the pores of a 25.4 mm diameter cylindrical sample 4 mm thick were filled with a wetting fluid having a surface tension of about 19 dynes/cm then that sample was loaded into a sample chamber with a microporous membrane, having pores about 27 μm in diameter, placed under the sample. Thereafter, the air pressure above the sample was increased slowly to extrude the liquid from the sample. For a low surface tension wetting fluid, such as the one used, the wetting liquid that spontaneously filled the pores of the sample also spontaneously filled the pores of the microporous membrane beneath the sample when the pressure above the sample began to increase. As the pressure continued to increase, the largest pores of the sample emptied earliest. Further increases in the pressure above the sample led to the empting of increasingly smaller sample pores as the pressure continued to increase. The displaced liquid passed through the membrane and its volume was measured. Thus, the volume of the displaced liquid allowed the internal volume accessible to the liquid, i.e., the liquid intrusion volume, to be obtained. Moreover, measurement of the liquid flow under increasing pressure but in the absence of the microporous membrane beneath the sample, this time using water as the fluid, allowed the liquid permeability to be determined. The liquid intrusion volume of the foam was determined to be 4 cc/g and the permeability of water through the foam was determined to be 1 L/min/psi/cc (0.00142 L/min/(kg/m²)/cc).

### Example 16

Reticulation of a Crosslinked Polyurethane Foam

Reticulation of the foam described in Example 15 was carried out by the following procedure. A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about 2 minutes to remove substantially all of the air in the foam. A mixture of hydrogen to oxygen gas, present at a ratio sufficient to support combustion, was charged into the chamber over a period of about 3 minutes. The gas in the chamber was then ignited by a spark plug. The ignition exploded the gas mixture within the foam. The explosion was believed to have blown out many of the cell walls between adjoining pores, thereby forming a reticulated elastomeric matrix structure.

Tensile tests were conducted on reticulated foam samples as described in Example 15. The average tensile strength was determined to be about 23.5 psi (about 16,450 kg/m²). The elongation to break was determined to be about 194%.

The post-reticulation compressive strength of the foam was determined as described in Example 15. The compressive strength at 50% compression was determined to be about 6.5 psi (about 4,550 kg/m²).

The pore structure and its inter-connectivity is characterized using a Liquid Extrusion Porosimeter as described in Example 15. The liquid intrusion volume of the reticulated foam was determined to be 28 cc/g and the permeability of water through the reticulated foam was determined to be 413 L/min/psi/cc (0.59 L/min/(kg/m²)/cc). These results demonstrate, e.g., the interconnectivity and continuous pore structure of the reticulated foam.

### Example 17

Fabrication of a Soft-Segment-Crosslinked Reticulated Polvurethane Matrix

A polymeric 4,4′-MDI with an isocyanate functionality of about 2.3 (PAPI 901, supplied by Dow) is used as the isocyanate component. Two polyether polyols, VORANOL 4703 and VORANOL 4925 (supplied by Dow), each approximately trifunctional, are used as the polyol component. The alkanol amine chain extender diethanolamine (supplied by Eastman Kodak Co.) is used. Water is used as the blowing agent. The blowing and gelling catalyst is a 2,2′-oxybis(N,N-dimethyl ethylamine)/glycol mixture (NIAX® A-1, supplied by OSI Specialties, Inc.). The blowing catalyst is the tertiary amine 33% triethylenediamine in dipropylene glycol (DABCO 33LV). A silicone-based surfactant is used (DC 5241, supplied by Dow Coming). The proportions of the components used is given in Table 5.

TABLE 5

| Ingredient | Parts by Weight |
|---|---|
| Polyol Component | |
| VORANOL 4703 Polyether Polyol | 50 |
| VORANOL 4925 Polyether Polyol | 50 |
| Isocyanate Component | As required for 1.05 |
| | Isocyanate Index |
| Isocyanate Index | 1.05 |
| Chain Extender | 1.5 |
| Water | 4.0 |
| Blowing and Gelling Catalyst | 0.15 |
| Blowing Catalyst | 0.45 |
| Surfactant | 1.0 |

To make the foam, all of the ingredients except the isocyanate component are first admixed. Then, the isocyanate component is added, with stirring, and the foaming mixture is poured into a cardboard form and allowed to rise.

The foam is reticulated by the procedure described in Example 13.

### Example 18

Fabrication of a Reticulated Polycarbonate Polyurethane Matrix by Lyophilization

A homogeneous solution of 10% by weight of BIONATE® 80A grade polycarbonate polyurethane in DMSO is prepared by tumbling and agitating the BIONATE pellets in the DMSO using a rotary spider turning at 5 rpm over a 3 day period. The solution is made in a sealed container to minimize solvent loss.

The solution is placed in a shallow plastic tray and held at 27° C. for 30 minutes. The lyophilizer tray temperature is dropped to −10° C. at a cooling rate of 1.0° C./minute and the pressure within the lyophilizer is reduced to 50 millitorr. After 24 hours, the temperature of the tray is raised at a rate of about 0.5° C./hour to 8° C. and held there for 24 hours. Then, the temperature of the tray is raised at a rate of about 1° C./hour until a temperature of 25° C. is reached. Then, the temperature of the tray is further raised at a rate of about 2.5° C./hour until a temperature of 35° C. is reached. During lyophilization, DMSO sublimes leaving a reticulated polycarbonate polyurethane matrix piece. The pressure is returned to 1 atmosphere and the piece is removed from the lyophilizer.

Any remaining DMSO is washed off of the piece by repeatedly rinsing it with water. The washed piece is allowed to air-dry.

Disclosures Incorporated

The entire disclosure of each and every U.S. patent and patent application, each foreign and international patent publication and each other publication, and each unpublished patent application that is referenced in this specification, or elsewhere in this patent application, is hereby specifically incorporated herein, in its entirety, by the respective specific reference that has been made thereto.

While illustrative embodiments of the invention have been described above, it is, of course, understood that many and various modifications will be apparent to those in the relevant art, or may become apparent as the art develops. Such modifications are contemplated as being within the spirit and scope of the invention or inventions disclosed in this specification.

1. An implantable device comprising a reticulated resiliently-compressible elastomeric matrix.

2. The implantable device of claim 1, wherein the implantable device is biodurable for at least 29 days.

3. The implantable device of claim 1, wherein the elastomeric matrix comprises a polycarbonate polyurethane.

4. The implantable device of claim 3, wherein the implantable device is biodurable for at least 6 months.

5. The implantable device of claim 1, comprising a reticulated elastomeric matrix comprising a plurality of pores, the pores having an average diameter or other largest transverse dimension of at least about 150 μm.

6. The implantable device of claim 3, wherein the pores have an average diameter or other largest transverse dimension of from greater than 250 μm to about 900 μm.

7. The implantable device of claim 1, comprising a reticulated elastomeric matrix comprising a plurality of pores, the pores having an average diameter or other largest transverse dimension of from about 275 μm to about 900 μm.

8. The implantable device of claim 1, comprising a reticulated elastomeric matrix comprising a plurality of pores, the pores having an average diameter or other largest transverse dimension of from about 275 μm to about 700 μm.

9. The implantable device of claim 1, comprising a resiliently-compressible elastomeric matrix such that the implantable device, when compressed from a relaxed configuration to a first, compact configuration for delivery via a delivery-device, expands to a second, working configuration, in vitro, at least about 80% of the size of the relaxed configuration in at least one dimension.

10. The implantable device of claim 9, wherein the recovery properties of the elastomeric matrix are such that a dimension of the second, working configuration is within about 20% of a relaxed dimension of the relaxed configuration after compression to from about 50 to about 10% of the relaxed dimension and wherein the elastomeric matrix has a compressive strength at 50% compression of from about 1 psi (about 700 kg/m²) to about 200 psi (about 140,000 kg/m²), a tensile strength of from about 1 psi (about 700 kg/m²) to about 75 psi (about 52,500 kg/m²) and an ultimate tensile elongation of at least about 150%.

11. The implantable device of claim 1, wherein the elastomeric matrix has a compression set after 22 hours compression at about 25° C. to 25% of its thickness in one dimension of not more than about 30%.

12. The implantable device of claim 1, wherein the elastomeric matrix comprises polycarbonate, polyether, polysiloxane, polyurethane, hydrocarbon, or mixtures thereof.

13. The implantable device of claim 1, wherein the reticulated elastomeric matrix is configured to permit cellular ingrowth and proliferation into the elastomeric matrix.

14. A process for producing an elastomeric matrix comprising a polymeric material having a reticulated structure, the process comprising:
a) fabricating a mold having surfaces defining a microstructural configuration for the elastomeric matrix;
b) charging the mold with a flowable polymeric material;
c) solidifying the polymeric material; and
d) removing the mold to yield the elastomeric matrix.

15. The process of claim 14, wherein the mold is a sacrificial mold and is removed by melting, dissolving or subliming the sacrificial mold.

16. The process of claim 14, wherein the sacrificial mold comprises a plurality of particles interconnected one with another at multiple points on each particle, wherein the flowable polymeric material is contained within the interstices between the particles.

17. The process of claim 16, wherein the particles comprise a first material having a melting point at least 5° C. lower than the softening temperature of the polymeric material that is contained within the interstices where, optionally, the first material comprises a hydrocarbon wax.

18. The process of claim 16, wherein the particles comprise an inorganic salt, a sugar, a starch, or mixtures thereof.

19. The process of claim 18, wherein the particles comprise starch and the starch is removed enzymatically.

20. The process of claim 18, wherein the polymeric material comprises a solvent-soluble thermoplastic elastomer, the flowable polymeric material comprises a solution of the thermoplastic elastomer in a solvent, and the solvent is evaporated to solidify the thermoplastic elastomer.

21. The process of claim 20, wherein the thermoplastic elastomer is selected from the group consisting of polycarbonate polyurethanes, polyether polyurethanes, polysiloxane polyurethanes, hydrocarbon polyurethanes, polyurethanes with mixed soft segments, and mixtures thereof.

22. A process for producing an elastomeric matrix having a reticulated structure, the process comprising:
a) coating a reticulated foam template with a flowable resistant material, optionally a thermoplastic polymer or a wax;
b) exposing a coated surface of the foam template;
c) removing the foam template to yield a casting of the reticulated foam template;
d) coating the casting with an elastomer in a flowable state to form an elastomeric matrix;
e) exposing a surface of the casting; and
f) removing the casting to yield a reticulated elastomeric matrix comprising the elastomer.

23. The process of claim 22, wherein the elastomer is a thermoplastic elastomer selected from the group consisting of polycarbonate polyurethanes, polyether polyurethanes, polysiloxane polyurethanes, hydrocarbon polyurethanes, polyurethanes with mixed soft segments, and mixtures thereof.

24. A lyophilization process for producing an elastomeric matrix having a reticulated structure, the process comprising:
a) forming a solution comprising a solvent-soluble biodurable elastomer in a solvent;
b) at least partially solidifying the solution to form a solid, optionally by cooling the solution; and
c) removing the non-polymeric material, optionally by subliming the solvent from the solid under reduced pressure, to provide an at least partially reticulated elastomeric matrix comprising the elastomer.

25. The process of claim 24, wherein the elastomer is a thermoplastic elastomer selected from the group consisting of polycarbonate polyurethanes, polyether polyurethanes, polysiloxane polyurethanes, hydrocarbon polyurethanes, polyurethanes with mixed soft segments, and mixtures thereof.

26. A polymerization process for preparing a reticulated elastomeric matrix, the process comprising admixing:
a) a polyol component,
b) an isocyanate component,
c) a blowing agent,
d) optionally, a crosslinking agent,
e) optionally, a chain extender,
f) optionally, at least one catalyst,
g) optionally, a surfactant, and
h) optionally, a viscosity modifier;
to provide a crosslinked elastomeric matrix and reticulating the elastomeric matrix by a reticulation process to provide the reticulated elastomeric matrix.

27. The process of claim 26, wherein the polyol component is liquefied prior to admixing.

28. The process of claim 27, wherein a first admixture comprising the polyol and isocyanate components is formed by admixing the polyol component and the isocyanate component; a second admixture comprising the blowing agent and, optionally, the catalyst is formed by admixing the blowing agent and the optional catalyst; and the first admixture and the second admixture are admixed.

29. The process of claim 26, wherein the polyol component comprises a polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, or mixtures thereof.

30. The process of claim 29, wherein the polyol component comprises a difunctional polycarbonate diol.

31. The process of claim 30, wherein the difunctional polycarbonate diol is 1,6-hexamethylene polycarbonate diol.

32. The process of claim 26, wherein the isocyanate component comprises tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate), p-phenylene diisocyanate, 4,4′-diphenylmethane diisocyanate, 2,4′-diphenylmethane diisocyanate, 2,4-toluene diisocyanate, 2,6-toluene diisocyanate, m-tetramethylxylene diisocyanate, or mixtures thereof.

33. The process of claim 32, wherein the isocyanate component comprises MDI, wherein the MDI is a mixture of at least about 5% by weight of 2,4′-MDI with the balance 4,4′-MDI.

34. The process of claim 32, wherein the average number of isocyanate groups per molecule in the isocyanate component is about 2.

35. The process of claim 32, wherein the average number of isocyanate groups per molecule in the isocyanate component is greater than 2.

36. The process of claim 35, wherein the average number of isocyanate groups per molecule in the isocyanate component is greater than about 2.2.

37. The process of claim 32, wherein the isocyanate component has an isocyanate index and wherein the isocyanate index is from about 0.9 to 1.029.

38. The process of claim 37, wherein the isocyanate index is from about 0.98 to about 1.02.

39. The process of claim 37, wherein the isocyanate index is from about 0.9 to about 1.1.

40. The process of claim 26, wherein the blowing agent is water.

41. The process of claim 26, wherein a tertiary amine is present as a catalyst.

42. The process of claim 26, wherein a silicone-based surfactant is present as a surfactant.

43. The process of claim 26, wherein propylene carbonate is present as a viscosity modifier.

44. The process of claim 26, wherein the reticulation is by combustion reticulation.

45. The process of claim 44, wherein the combustible atmosphere comprises a mixture of hydrogen and oxygen.

46. A process for preparing a reticulated composite elastomeric implantable device, the process comprising endoporously coating a reticulated elastomeric matrix with a coating material selected to encourage cellular ingrowth and proliferation.

47. The process of claim 46, wherein the coating material comprises a foamed coating of a biodegradable material, the biodegradable material comprising collagen, fibronectin, elastin, hyaluronic acid or mixtures thereof.

48. A method of treating a vascular malformation, the method comprising:
a) compressing the implantable device of claim 1 from a relaxed configuration to a first, compact configuration;
b) delivering the compressed implantable device to the in vivo site of the vascular malformation via a delivery-device; and
c) allowing the implantable device to expand to a second, working configuration at the in vivo site.

49. The method of claim 48, wherein the implantable device comprises a plurality of elastomeric matrices.
